(19) United States
(12) Patent Application Publication
(10) Pub. No.: US 2019/0284566 A1
(43) Pub. Date: Sep. 19, 2019

(54) WHEAT

(71) Applicant: ELSOMS DEVELOPMENTS LTD, Spalding, Lincolnshire (GB)

(72) Inventors: Anthony Gordon KEELING, Spalding, Lincolnshire (GB); Matthew John MILNER, Cambridge, Cambridgeshire (GB)

(73) Assignee: ELSOMS DEVELOPMENTS LTD, Spalding, Lincolnshire (GB)

(21) Appl. No.: 16/320,146

(22) PCT Filed: Jul. 20, 2017
(86) PCT No.: PCT/US2017/043009

§ 371 Date: Jan. 24, 2019

Related U.S. Application Data
(60) Provisional application No. 62/453,115, filed on Feb. 1, 2017.
(60) Provisional application No. 62/436,678, filed on Dec. 20, 2016.

(30) Foreign Application Priority Data
Jul. 29, 2016 (GB) 1613156.7

(51) Int. Cl. C12N 15/82; C12N 15/11; C12N 9/22

(52) U.S. Cl. CPC C12N 15/8218; C12N 15/11; C12N 2800/80; C12N 15/8289; C12N 2310/20; C12N 9/22

(57) ABSTRACT

Described herein are methods and compositions relating to male-sterile wheat, as well as uses thereof and methods for propagating and maintaining the same.

### CROSS-REFERENCE TO RELATED APPLICATIONS

This application claims benefit under 35 U.S.C. § 119(e) of U.S. Provisional Application Nos. 62/436,678 filed Dec. 20, 2016 and 62/453,115 filed Feb. 1, 2017 and which claims the benefit of foreign priority under 35 U.S.C. § 119(a) of UK provisional application No. 1613156.7 filed Jul. 29, 2016, the contents of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Jul. 19, 2017, is named 077524-088582_SL.txt and is 617,500 bytes in size.

### TECHNICAL FIELD

The invention relates to wheat, more particularly to male-sterile wheat and methods of producing and using it. More specifically, the invention relates to methods of producing wheat plants exhibiting genetic male-sterility (GMS), in particular by inhibiting certain wheat genes: materials useful in such methods; plants and plant populations obtainable by such methods; as well as to F1 hybrids obtainable by crossing such plants with male-fertile wheat. Wheat genes whose inhibition results in male-sterility in wheat are referred to herein as male-fertility wheat (Mfw) genes.

### BACKGROUND

Plants produce seed by the union of male and female gametes. The male gametes are carried in pollen, the female gametes in ovules. Many crop species are largely self-sterile, meaning that the progeny of a plant are mostly outcrosses, produced by cross-pollination with another plant. However, certain crop species are capable of self-pollination, as well as cross-pollination. Some self-fertile crops, among them wheat, are usually self-pollinators. Hybrid breeding systems have been developed for certain crops (one example is sugar beet) to enable a parent line without pollen to be cross-pollinated by a pollen-producing line in the seed production field thus producing F₁ seed. However many such hybrid systems do not require male-fertility, because the commercial product of the F₁ is (or is from) the vegetative part of the plant. F₁ plants of grain crops such as wheat must have their male-fertility restored in order to produce saleable grain.

Hybrid plant breeding has led to major improvements in crop yield due primarily to the benefits associated with heterosis (hybrid vigour) in F₁ hybrid plants. Development of hybrid breeding systems is, therefore, highly desirable. Also, since the parent lines most suitable for generating F₁ hybrid seed are usually not made freely available to the market, F₁ hybrids offer the plant breeder a more controllable and profitable business model, driving further development of new breeding systems, with benefits for plant breeders, farmers and consumers.

### SUMMARY

At present, there are no convenient and readily practicable methods of producing male-sterile wheat (common wheat, Triticum aestivum)—see Whitford et al (2013). The present invention provides a new method of obtaining male-sterile wheat, which avoids at least some of the inconveniences associated with or foreseeable with previously proposed methods. It further provides new male-sterile wheat plants that may be obtained by the process of the invention, and new hybrids made by crossing such male-sterile wheat with male-fertile wheat.

### FIGURES

FIG. 1 shows amino-acid sequences SEQ ID NOs 1, 2 and 3.

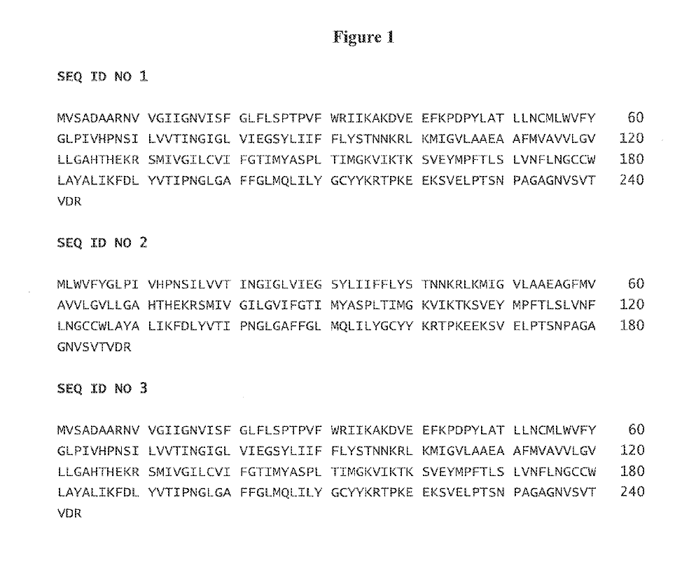

FIG. 2 shows amino-acid sequences SEQ ID NOs 4 and 5.

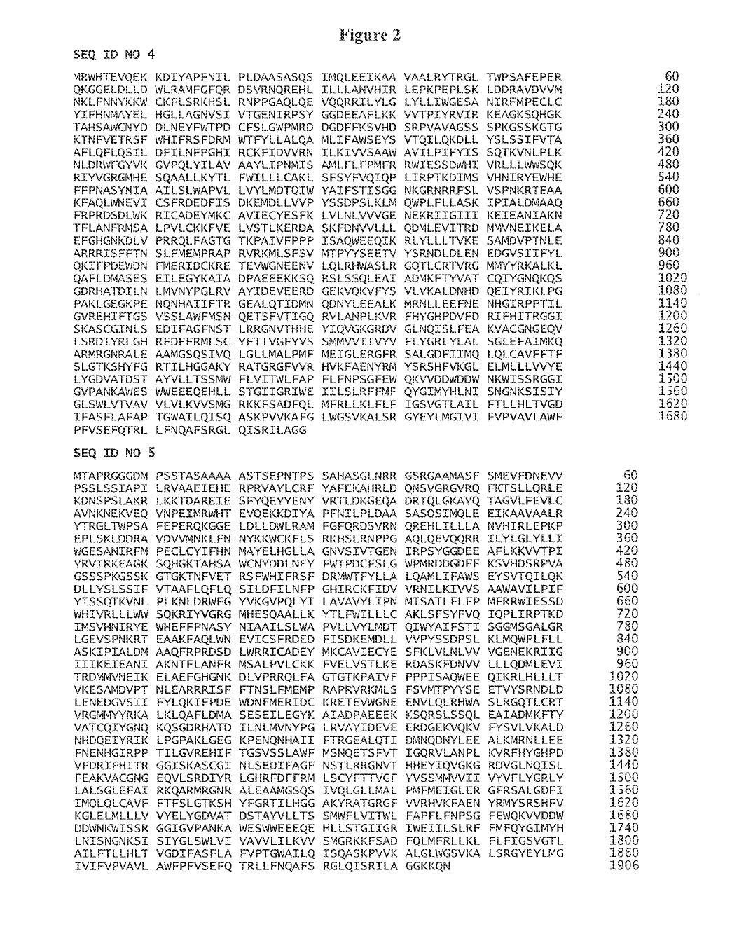

FIG. 3 shows amino-acid sequence SEQ ID NO 6 and DNA sequence SEQ ID NO 7.

FIG. 4 shows DNA sequence SEQ ID NO 10 (bases 1-3540).

FIG. 5 shows DNA sequence SEQ ID NO 10 (bases 3541-5127).

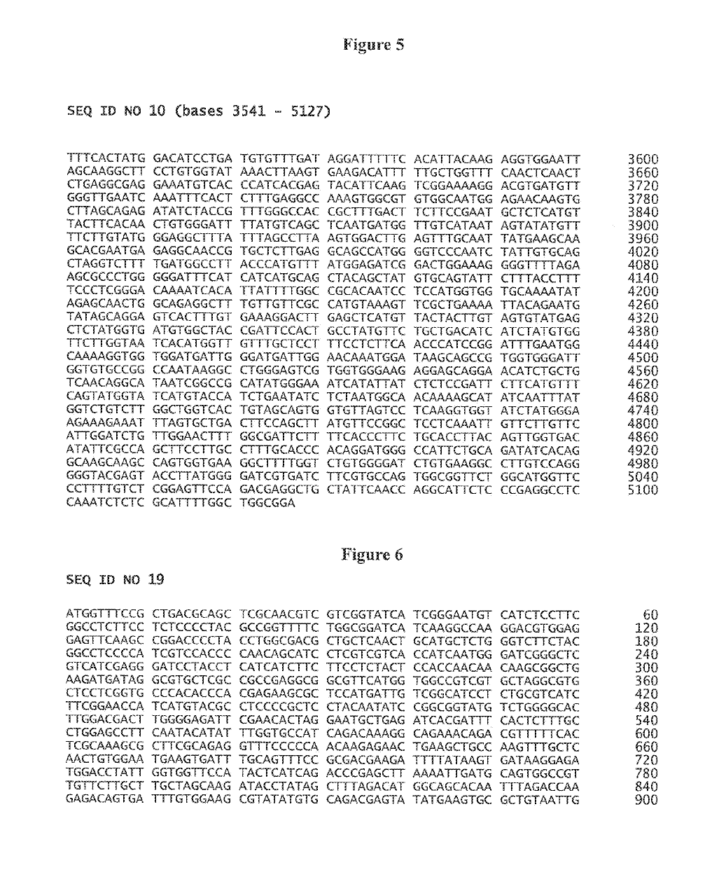

FIG. 6 shows the base sequence of the DNA insert to be introduced into the wheat genome in Example 2.

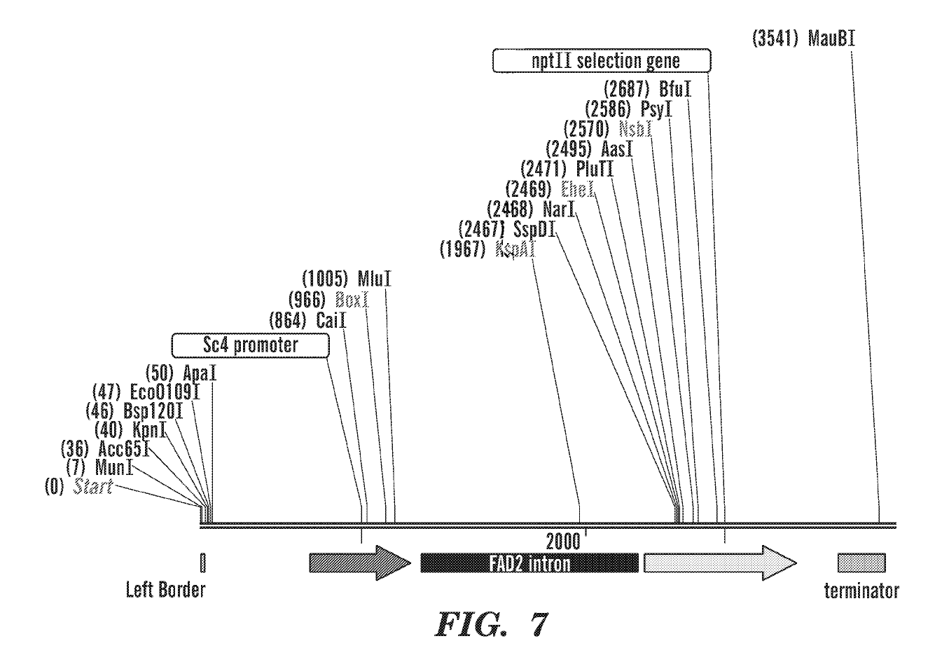

FIGS. 7 and 8 together show a schematic map of the construct used to insert the base sequence of FIG. 4 into the wheat genome; and the following Examples 1-4.

FIG. 9 depicts a schematic of an exemplary approach to generating a male-sterile wheat plant utilizing CRISPR/Cas. When the resulting plant is pollinated by a wild-type wheat plant, a male-fertile F1 hybrid will result. Figure discloses SEQ ID NOS 51-53, respectively, in order of appearance.

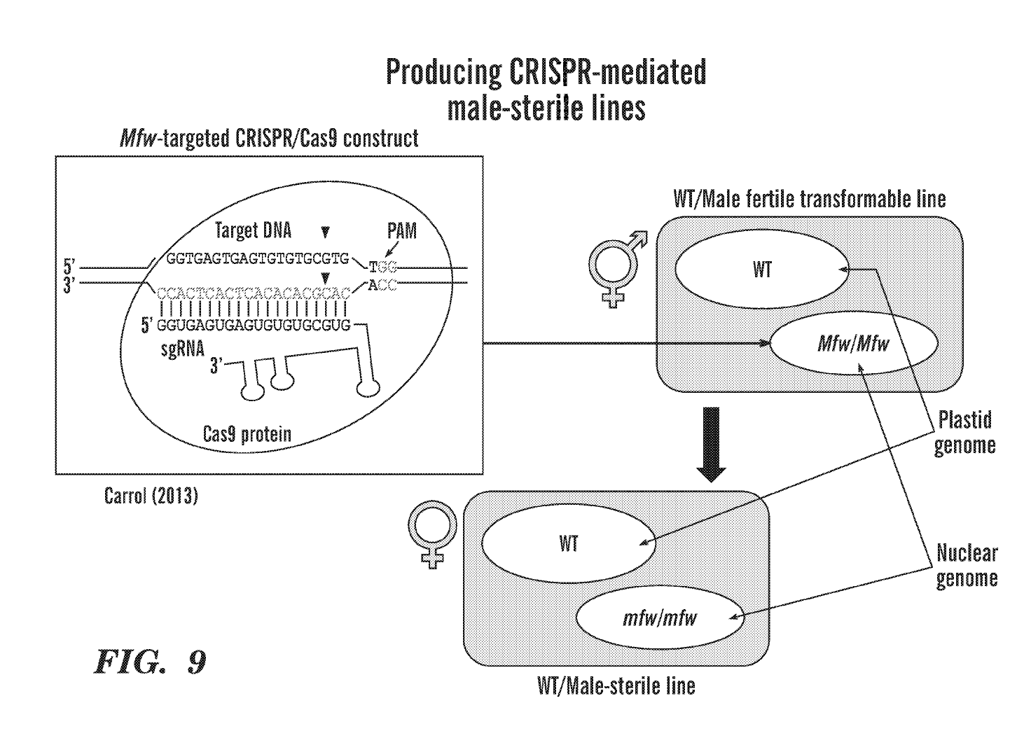

FIG. 10 depicts a schematic of an exemplary approach for a cytoplasmic-genome male-fertility-restorer gene system as a pollen source to maintain a male-sterile wheat plant.

FIGS. 11 and 12 depict schematics of an exemplary nuclear-genome approach to producing and maintaining a male-sterile wheat plant.

FIG. 13 depicts a schematic of an exemplary approach to reproducing a nuclear-genome or genic “maintainer/maintainer-line” for a male-sterile wheat plant.

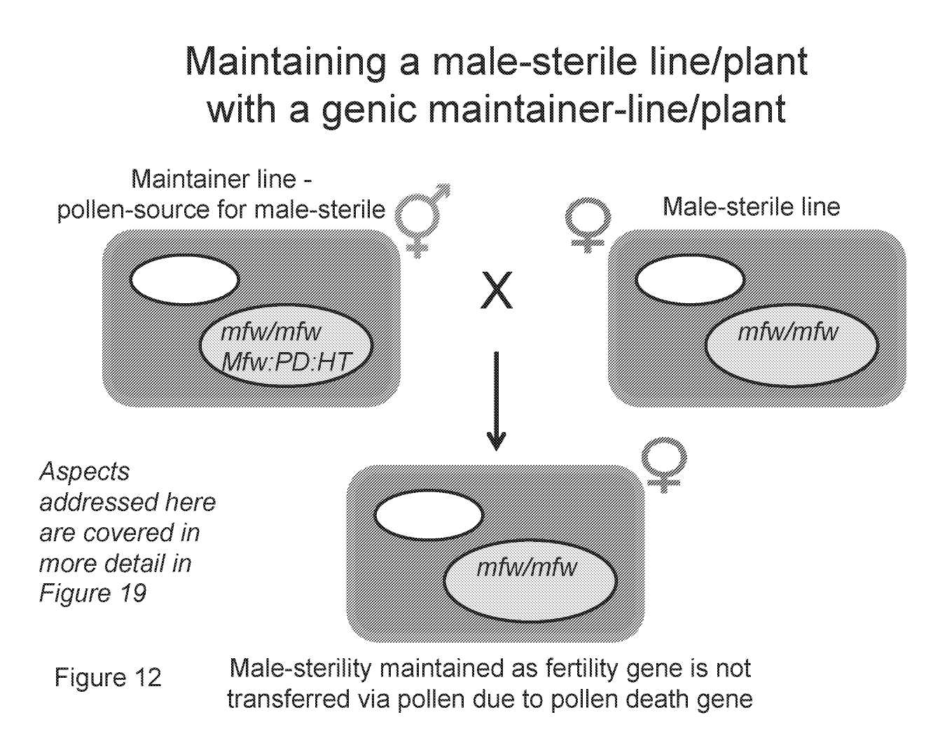

FIG. 14 depicts a schematic of an exemplary approach to reproducing a cytoplasmic-genome “maintainer-line” for a male-sterile wheat plant.

FIG. 15 depicts a schematic of an exemplary approach to crossing a male-sterile wheat plant produced by Mfw gene knock-out, eg by CRISPR, to produce fertile F1 hybrid plants.

FIG. 16 depicts a schematic of an exemplary approach to transferring male-sterility by conventional breeding.

FIG. 17 depicts Alexander staining and FIG. 18 depicts Auramine O staining of control pollen and a plant in which Mfw1 and Mfw2 have been deactivated by RNAi silencing.

FIGS. 17A-17J depict images of pollen from RNAi plant 27 (FIGS. 17A-17E) or wild type pollen (FIGS. 17F-17J) stained with Alexander stain (FIGS. 17A, 17B, 17F, 17G) or Auramin O (FIGS. 17C-17E, 17H-17J). All pictures are shown at 100× except for 17E and 17J which are shown at 400×

FIG. 18 depicts a schematic of genetic events taking place in a genic maintainer line.

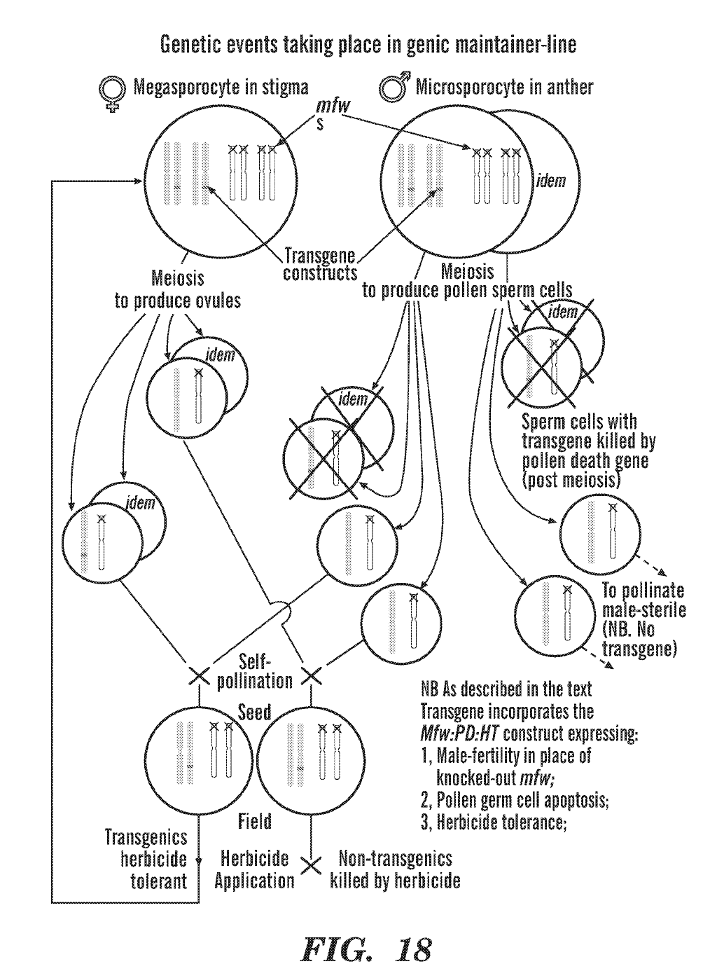

### DETAILED DESCRIPTION

Our invention includes a method of producing male-sterile wheat which comprises during the development of the wheat flower:

analysing the RNA-transcriptome of wheat stamen cells;

analysing the RNA-transcriptome of wheat pistil cells;

then comparing the two RNA-transcriptomes to identify one or more genes that at the time of flowering are preferentially expressed in stamens rather than pistils;

selecting one or more genes so identified; and

inhibiting expression of selected genes, so as to produce male-sterile wheat.

Relative transcript abundance analysis is carried out on RNA collected preferably during early-stage development of the flower, in particular during meiosis, which occurs during development of the gametes in the wheat flower as it develops while still inside the stem of the wheat plant; this can be defined as between stages 41 to 49 of the Zadoks scale, inclusive—see Zadoks et al, (1974). Wheat is hexaploid, and in many varieties/cultivars it is found that the same, or substantially the same, Mfw gene occurs more than once in the genome: in one or more of the three sets of homoeologous chromosomes. In such cases, in order to obtain male-sterile wheat, it may be necessary to deactivate this gene at each of the three loci on the homoeologous chromosomes where the gene is present. The precise loci needing to be deactivated are found by examination of plants which have had different homoeologues of the Mfw genes deactivated. (This will be evident in plants which have all homoeologs deactived after gene-editing.)

Others working in this field have worked with male-fertility genes which have clearly and effectively expressed male-fertility/-sterility in other monocot species and then tried to find orthologues expressing a male-fertility phenotype in wheat. To date, this approach has not been successful.

Additionally, many prior approaches to male-sterility involve temperature sensitivity and/or cytoplasmic male sterility (CMS). These approaches are marked by reduced yields and/or “leaky” phenotypes which render them unsuitable for commercial uses, particularly in wheat.

In contrast, the methods described herein relate to identifying genes which are expressed specifically and substantially in the wheat plant at or about meiosis (e.g., during Zadoks stages 41-49, inclusive), when the genes which are vital to pollen development and function are needed to be expressed for proper pollen development and function. In accordance with some embodiments of the invention, this range of developmental stages was identified since it encompasses expression of genes associated with pollen development and function. Also, the ear first matures in the middle and then matures to both tip and base (Zadok et. al, 1974). So, to limit the range of microsporogenesis stages in the samples to meiosis or slightly pre- or post-meiosis, juvenile flowers were selected from this middle part of the range in which immature stamens and pistils were present. Wheat, with an estimated 104,000 protein-coding genes, (see Clavijo et al, (2016)) has a large transcriptome with a polyploid genome and it is part of our invention to take this complexity into account by focusing solely on genes required for pollen development in wheat plants. Notably, forward genetic approaches (e.g., random mutagenesis followed by a survey of resulting phenotypes) are thus of minimal use in the complex genome of wheat, particularly as compared to other crop plants.

The first step of our process identifies a considerable number of genes that are preferentially expressed in wheat stamens. It is generally impractical to inhibit all of these, so a further selection is made. This may be based on a wide variety of factors. These include preferences for:
genes having homology with genes from other species previously described as being involved with pollen development or male fertility;
genes whose function in pollen development or male fertility may be inferred from their sequence;
genes that are conserved within and across species (autologous and paralogous conservation);
genes having a demonstrated male-sterile phenotype in plants.
   Practical factors may also be taken into account, such as availability and cost. A final selection may be made of genes that have homoeologous copies in at least two and preferably three out of the three wheat genomes.

Wheat genes whose inhibition results in male-sterility in wheat we term male-fertility wheat (Mfw) genes. If Mfw genes are missing from a wheat plant, or are inactive/deactivated, the wheat plant will show reduced fertility. Mfw genes may be identified by the process of our invention. Exemplary non-limiting examples of Mfw genes are provided in Table 1 and Table 2.

In one aspect of any of the embodiments, described herein is a method of producing male-sterile wheat, the method comprising inhibiting expression of at least one Mfw gene. In one aspect of any of the embodiments, described herein is a wheat plant or seed, or population of wheat plants and/or seeds which is predominantly male-sterile and comprises one or more deactivated Mfw genes. In one aspect of any of the embodiments, described herein is a process of obtaining wheat hybrids, the method comprising crossing a population which is predominantly male-sterile and comprises one or more deactivated Mfw genes with pollen from male-fertile wheat. In one aspect of any of the embodiments, described herein is a hybrid or population of hybrids produced by crossing a population which is predominantly male-sterile and comprises one or more deactivated Mfw genes with male-fertile wheat.

In some embodiments of any of the aspects, a gene can be preferentially expressed in wheat stamens as compared to wheat pistils. Genes with such an expression pattern are referred to herein as male-fertility preferential expression in wheat (Mpew) genes. In some embodiments of any of the aspects, the expression level of a given gene in wheat stamens and pistils can be the expression level occurring between stages 41 to 49 of the Zadoks scale, inclusive. In some embodiments of any of the aspects, the expression level of a given gene in wheat stamens and pistils can be the expression level occurring during or about meiosis. In some embodiments of any of the aspects, the expression level of a given gene in wheat stamens and pistils can be the expression level occurring during meiosis. In some embodiments of any of the aspects, preferentially expressed refers to an expression level which is at least 1.5×, e.g., at least 2×, at least 2.5×, at least 3×, at least 5×, at least 10×, at least 20×, at least 30×, at least 50×, at least 100×, or greater in the preferred tissue as compared to the reference tissue (e.g., in wheat stamens as compared to wheat pistils).

In one aspect of any of the embodiments, described herein is a method of producing male-sterile wheat, the method comprising inhibiting expression of at least one Mpew gene. In one aspect of any of the embodiments, described herein is a wheat plant or seed, or population of wheat plants and/or seeds which is predominantly male-sterile and comprises one or more deactivated Mpew genes. In one aspect of any of the embodiments, described herein is a process of obtaining wheat hybrids, the method comprising crossing a population which is predominantly male-sterile and comprises one or more deactivated Mpew genes with male-fertile wheat. In one aspect of any of the embodiments, described herein is a hybrid or population of hybrids produced by crossing a population which is predominantly male-sterile and comprises one or more deactivated Mpew genes with male-fertile wheat.

In some embodiments of any of the aspects, a gene can be both a Mfw and an Mpew gene, e.g., the gene can be preferentially expressed in wheat stamens versus wheat pistils and when deactivated, the gene results in wheat male-sterility (e.g., a Mfw/Mpew gene). In any embodiment of a method or composition in which reference to a Mfw gene is made herein, alternative embodiments comprising a Mpew and/or an Mfw/Mpew gene are specifically contemplated. Our invention includes male-infertile wheat plants containing one or more Mfw genes identified by the process of the invention as important to the callose-synthesis aspect of male-fertility, expression of which has been inhibited. Such specific Mfw genes (Mfw2-A, Mfw2-B and Mfw2-D) include those having gene sequences corresponding to those shown in SEQ ID NOs 7-12, and genes having at least 90% and preferably at least 95% or 97% identity therewith. The invention further includes male-infertile wheat plants in which a selected Mfw gene codes for an amino-acid sequence identical, or having corresponding function and least 80%, preferably 95% or 97% identity, with any of SEQ ID NOs 1-6.

In some embodiments of any of the aspects, a Mfw and/or Mpew gene can be a gene selected from Table 1 or 2. In some embodiments of any of the aspects, a Mfw and/or Mpew gene can be a homolog, ortholog, and/or variant of a gene selected from Table 1 or 2. In some embodiments of any of the aspects, a Mfw and/or Mpew gene can be a gene with at least 90%, at least 95%, at least 97% or greater amino acid sequence identity with a gene selected from Table 1 or 2. In some embodiments of any of the aspects, a Mfw and/or Mpew gene can be a gene with at least 90%, at least 95%, at least 97% or greater nucleic acid sequence identity with a gene selected from Table 1 or 2.

The sequences provided in Tables 1 and 2 are the sequences for the identified genes in the Fielder variety of wheat. In some embodiments of any of the aspects, a Mfw and/or Mpew gene can be the gene from a wheat variety other than Fielder which has the highest degree of homology and/or sequence identity with a gene selected from Table 1 or 2. In some embodiments of any of the aspects, a Mfw and/or Mpew gene can be the gene from a wheat variety other than Fielder which has the greatest degree of homology and/or sequence identity with a gene selected from Table 1 or 2.

Examples of specific Mfw genes that we have identified by the process of the invention are Mfw1 genes, Mfw2 genes, Mfw3 and Mfw5 genes. Mfw1 genes have homology with the gene for Ruptured Pollen Grain 1 (RPG1) (Sun M-X et al, 2013); Mfw2 genes with the gene for Callose Synthase (CalS5) (Dong et al., 2006). Both RPG1 and CalS5 are known genes in other non-cereal plant species that have been found to be involved in pollen formation. While others have found sequences in the Triticum genus that resemble genes in Table 1, no phenotypic evidence of a role in wheat plant male sterility for any of the Mfw genes described herein, nor sequences related thereto exists to date. Provided herein is such evidence of the function of certain genes in male sterility, e.g., for their use in hybrid wheat production.

Both Mfw1 and Mfw2 are found on each of the three sets of homoeologous chromosomes of wheat; we term these Mfw1-A, Mfw1-B, Mfw1-D, Mfw2-A, Mfw2-B and Mfw2-D according to the wheat genome (A, B or D) in which they have been found. The amino-acid sequence for which Mfw1-A codes is shown in SEQ ID NO: 01, Mfw1-B in SEQ ID NO: 02, Mfw1-D in SEQ ID NO: 03 and the amino-acid sequence for which Mfw2-A codes is shown in SEQ ID NO: 04, Mfw2-B in SEQ ID NO: 05 and Mfw2-D in SEQ ID NO: 06. The amino acid sequence for which Mfw3-A codes is shown in SEQ ID NO: 30. The amino acid sequence for which Mfw3-B codes is shown in SEQ ID NO: 31. The amino acid sequence for which Mfw3-D codes is shown in SEQ ID NO: 32. The amino acid sequence for which Mfw5-A codes is shown in SEQ ID NO: 33. The amino acid sequence for which Mfw5-B codes is shown in SEQ ID NO: 34. The amino acid sequence for which Mfw5-D codes is shown in SEQ ID NO: 35.

In some embodiments, the one or more Mfw and/or Mpew genes are: Mfw1 andMfw2; Mfw1 andMfw3; Mfw1 andMfw5; Mfw2 andMfw3; Mfw2 andMfw5; Mfw3 andMfw5; Mfw1, Mfw2, and Mfw3; Mfw1, Mfw2 and Mfw5; Mfw1, Mfw3 and Mfw5; Mfw2, Mfw3, and Mfw5; or Mfw1, Mfw2, Mfw3 andMfw5.

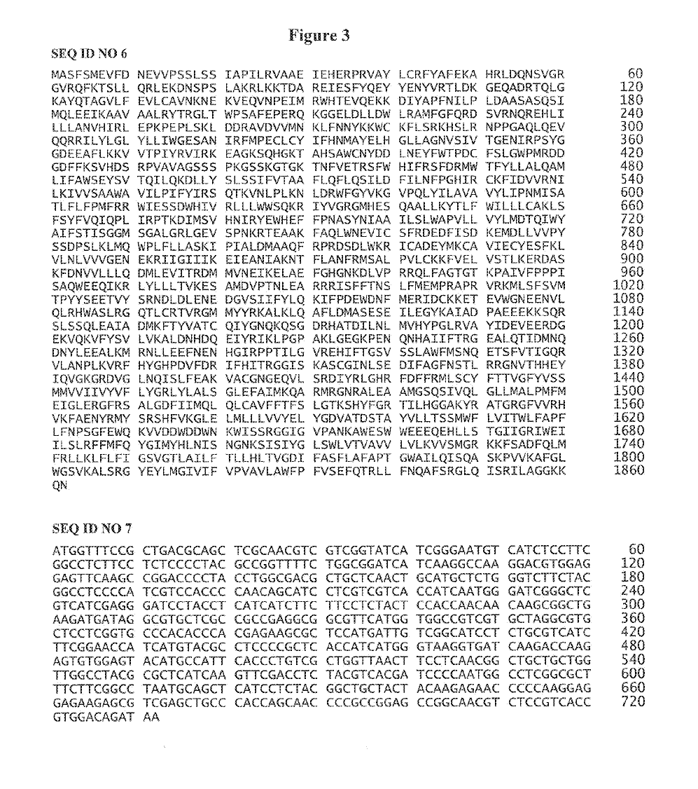

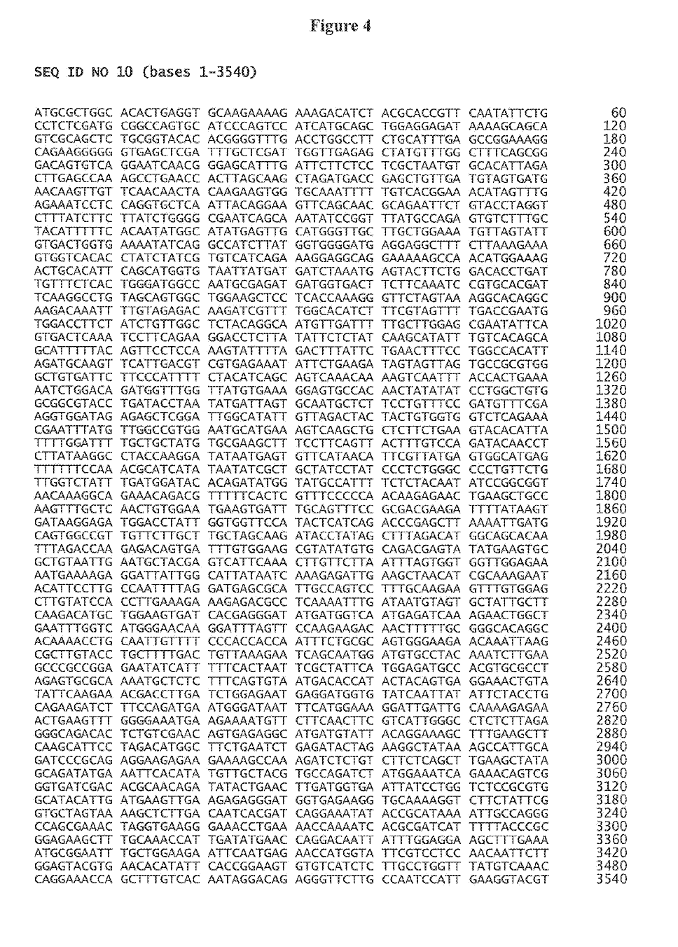

Our invention includes a process of producing male-sterile wheat which comprises inhibiting expression of Mfw genes that code for any of the amino-acid sequences shown in FIGS. 3 and 4, SEQ ID NOs 1-6 and/or 30-35 or for amino-acid sequences of corresponding function that have at least 60% and preferably at least 90%, particularly at least 95% sequence identity with those amino-acid sequences. % Sequence identity is the percentage of characters that match exactly when a first sequence is compared with a second sequence of the same or longer length. Gaps are not counted.

Percent identity of two proteins may be determined by comparison using available software tools, eg ‘BLAST’.

Our invention further provides a population of wheat plants that are male-sterile in consequence of the non-expression of at least one Mfw gene that is necessary for viable pollen production. Preferably the population comprises at least 50%, particularly 90%, 95% or 99%, of substantially genetically-uniform pollen-sterile seeds. Within the term ‘plants’ in this specification we include seeds and seedlings.

In one aspect, described herein is a population of wheat plants that are male sterile and comprising a deactivated Mfw and/or Mpew gene as described herein and/or or comprising a deactivating modification of a Mfw and/or Mpew gene as described herein. In some embodiments of any of the aspects, the population is substantially genetically uniform. In some embodiments of any of the aspects, the population is substantially genetically uniform at the locus and/or loci at which deactivating modifications have been made. In some embodiments of any of the aspects, the population is substantially genetically identical at each copy of the locus and/or loci at which deactivating modifications have been made. In some embodiments of any of the aspects, the population is genetically identical at the locus and/or loci at which deactivating modifications have been made. In some embodiments of any of the aspects, the population is genetically identical at each copy of the locus and/or loci at which deactivating modifications have been made. In some embodiments of any of the aspects, the population consists of individuals of the same genetic background, line and/or variety.

Another aspect of the present invention provides a process for producing a pollen-sterile wheat plant from a pollen-fertile wheat plant having an Mfw and/or Mpew gene, the process comprising deactivating an Mfw and/or Mpew gene of the pollen-fertile wheat plant. As used herein, a “deactivated” gene is one that, due to engineering and/or modification of the genome (both chromosomal and/or extrachromosomal) of the cell in which the gene is found, is expressed at less than 35% of the wild-type level of functional polypeptide. In some embodiments of any of the aspects, a deactivated gene is expressed at less than 30% of the wild-type level of functional polypeptide. In some embodiments of any of the aspects, a deactivated gene is expressed at less than 25% of the wild-type level of functional polypeptide. In some embodiments of any of the aspects, a deactivated gene is expressed at less than 20% of the wild-type level of functional polypeptide. In some embodiments of any of the aspects, a deactivated gene is expressed at less than 15% of the wild-type level of functional polypeptide.

The wild-type level of functional polypeptide can be the level of functional polypeptide found in the same type of cell not comprising the modification. In some embodiments of any of the aspects, the level of functional polypeptide can be the level of full-length polypeptide with a wild-type sequence.

In some embodiments of any of the aspects, deactivation of a gene can comprise engineering, modifying, and/or altering the genome of the cell in which the gene is found such that the cell expresses no more than 35% of the wild-type level of the polypeptide, inclusive of both full-length and partial sequences of the gene. In some embodiments of any of the aspects, a deactivated gene is expressed at less than 30% of the wild-type level of polypeptide, inclusive of both full-length and partial sequences of the gene. In some embodiments of any of the aspects, a deactivated gene is expressed at less than 25% of the wild-type level of polypeptide, inclusive of both full-length and partial sequences of the gene. In some embodiments of any of the aspects, a deactivated gene is expressed at less than 20% of the wild-type level of polypeptide, inclusive of both full-length and partial sequences of the gene. In some embodiments of any of the aspects, a deactivated gene is expressed at less than 15% of the wild-type level of polypeptide, inclusive of both full-length and partial sequences of the gene.

In some embodiments of any of the aspects, deactivation of a gene can comprise engineering, modifying, and/or altering the genome of the cell in which the gene is found such that the cell expresses polypeptides comprising no more than 35% of the wild-type sequence of the polypeptide. In some embodiments of any of the aspects, deactivation of a gene can comprise engineering, modifying, and/or altering the genome of the cell in which the gene is found such that the cell expresses polypeptides comprising no more than 30% of the wild-type sequence of the polypeptide. In some embodiments of any of the aspects, deactivation of a gene can comprise engineering, modifying, and/or altering the genome of the cell in which the gene is found such that the cell expresses polypeptides comprising no more than 25% of the wild-type sequence of the polypeptide. In some embodiments of any of the aspects, deactivation of a gene can comprise engineering, modifying, and/or altering the genome of the cell in which the gene is found such that the cell expresses polypeptides comprising no more than 20% of the wild-type sequence of the polypeptide. In some embodiments of any of the aspects, deactivation of a gene can comprise engineering, modifying, and/or altering the genome of the cell in which the gene is found such that the cell expresses polypeptides comprising no more than 15% of the wild-type sequence of the polypeptide. In some embodiments of any of the aspects, deactivation of a gene can comprise engineering, modifying, and/or altering the genome of the cell in which the gene is found such that the cell expresses polypeptides comprising no more than 10% of the wild-type sequence of the polypeptide. The invention further contemplates crossing male-sterile wheat obtainable by the process of the invention with male-fertile wheat to produce F1 hybrids, as well as hybrids so produced. A significant advantage of our invention is that it can, using gene editing technology, knockout Mfw genes and produce a recessive male-sterility genotype, mfw/mfw. This can allow F1 hybrids to be made by pollination with a wide range of wild-type male-fertile wheats that have endogenous dominant male-fertility Mfw/Mfw genes. In the next generation, such F1 hybrids resulting from our invention, are heterozygous Mfw/mfw, and so are fertile due to the dominance of the wild-type Mfw allele. In contrast, in some other hybrid systems, male-fertile pollinator lines need to be specially bred to incorporate a gene to restore fertility in the next generation, i.e., in the F1 plants in farmer-customers' fields (Whitford et al, 2013).

In some embodiments of any of the aspects, a population of plants as described herein can be at least 97% male-sterile, e.g., at least 97% male-sterile, at least 98% male-sterile, at least 99% male sterile, or 100% male-sterile. In some embodiments of any of the aspects, a population of plants as described herein can be at least 98% male-sterile. In some embodiments of any of the aspects, a population of plants as described herein can be at least 99% male-sterile. In some embodiments of any of the aspects, a population of plants as described herein can be 100% male-sterile. Male-sterile phenotypes described in other species can be of commercial value with even a partial male-sterility phenotype. Furthermore male-fertility genes in such other species, particularly diploid species, which have been mutated may be expected to express a male-sterility phenotype. If, as is often the case, those other plants species are 1) prone to cross-pollinate and/or 2) self-pollination is readily reduced or inhibited (e.g., detasseling of corn plants) a larger element of male-fertility may be acceptable in a male-sterile-based hybrid system in such species. In contrast, male-sterile wheat plants must demonstrate a phenotype that is significantly less “leaky” than what can be tolerated in other crops because wheat plants are much more likely to self-pollinate than other crop plants and physical interference with self-pollination is not practicable.

In some embodiments of any of the aspects, the male-sterile plants and/or hybrid plants described herein have a yield which is no less than 90% of the yield of a wild-type wheat plant of the same strain. In some embodiments of any of the aspects, the male-sterile plants and/or hybrid plants described herein have a yield which is no less than 95% of the yield of a wild-type wheat plant of the same strain. In some embodiments of any of the aspects, the male-sterile plants and/or hybrid plants described herein have a yield which is no less than 98% of the yield of a wild-type wheat plant of the same strain. Inhibition of Mfw genes may be carried out in various ways. Preferably inhibition of Mfw genes is carried out by targeted modification of the wheat genome, by additions or by deletions or by a combination of the two. Two main ways visualised by the invention are: by modifying the wheat genome so as to express RNA that inhibits expression of the identified Mfw gene; or by gene-editing to prevent the Mfw gene carrying out its function.

The transcriptome of a group of cells is the set of all RNA fragments generated in the cells at a particular time, including information about their relative abundance. It may be generated in various ways, in particular by DNA microarrays, or more preferably by the known technique of RNA-seq (whole transcriptome shotgun sequencing). This technique is described in more detail in Trick et al., (2012) and Harrison et al., (2015).

The whole wheat genome has previously been sequenced, and published. Sequences are given in Chapman et al (2014) and Clavijo et al, (2016) and were downloadable from, e.g., TGAC, The Genome Analysis Centre, Norwich in January 2016 and subsequently published in October 2016 as part of Clavijo et al., 2016. (available on the world wide web at ftp.ensemblgenomes.org/pub/plants/pre/fasta/triticum_aestivum/dna/). We have also sequenced the coding sequences for Mfw1 and Mfw2 in each of the three chromosome pairs of hexaploid wheat from the variety Fielder. These are shown in SEQ ID NOs 7-12 below. Our ‘Fielder’ sequences are very similar to but not identical with those obtained by TGAC (analysing variety Chinese Spring), Clavijo et al, (2016), and Chapman et al (2014) (which in turn differ slightly from each other). This is inevitable. Modern gene sequencing methods have a low but finite error rate—also the samples of wheat being sequenced may themselves have minor differences amongst and within different varieties. In selecting sequences of Mfw genes for use in the present invention, suitable coding sequences as shown as part of any of SEQ ID NOs 7-12 are preferred, but sequences from Clavijo et al, (2016), Chapman et al (2014) or TGAC (or any other academic publication) may also be useful. Further, Mfw genes may be inactivated by editing or deleting their associated promoter sequences. For example, the expression of Mfw1-A in variety Chinese Spring may be inhibited by editing of bases upstream (5′) of the start codon ATG at position 6072 of SEQ ID NO 13 so as to disrupt the action of the gene promoter. The position and number of the bases that must be removed, inserted or replaced so as to disrupt the action of the gene promoter may be determined by trial and error.

Individual modifications may be referred to herein as “deactivating modifications.” The phrase “deactivating modification” refers to a modification of an individual nucleic acid sequence and/or copy of a gene, which may or may not, on its own, result in deactivation of the desired gene. For example, deactivating modifications at all six copies of a given gene may be necessary to deactivate the gene. Furthermore, it is contemplated herein that the deactivating modification found at any given copy of a gene may or may not be identical to the deactivating modification found at the remaining copies of that gene.

In the context of a type of modification that is made at a location in the genome other than at the gene to be deactivated, a single modification may be sufficient to deactivate the gene (e.g, the introduction of an inhibitory nucleic acid). However, multiple copies of such modifications, at additional alleles and/or loci may be desirable to prevent “leaky”, imperfect or unreliable phenotype or prevent loss of the desired phenotypes in subsequent generations.

In the context of a type of modification that is made at the gene to be deactivated, e.g, an indel at the coding sequence of the gene, it can be necessary to introduce deactivating modifications at additional copies of the gene (e.g., at all six copies of a given homoeologous gene set in wheat) in order to effect deactivation of the gene. Accordingly, a modification at the gene to be deactivated is considered a deactivating modification if it deactivates the copy of the gene in which it occurs, regardless of its effect on other copies of the gene.

The inhibition and/or deactivation of an Mfw and/or Mpew gene, e.g., one identified according to the invention may be carried out by generation of interfering mRNA (RNAi). For example, the Mfw gene may be deactivated by RNAi repression, e.g., from an introgressed transgene designed for this purpose. An instance of this technique is illustrated in Example 3 below. Or deactivation may be by another form of genetic modification—for example by expressing a second copy of the relevant gene (or part of it) in reverse, to silence the gene.

In some embodiments of any of the aspects, a deactivating modification can be a modification that introduces an inhibitory nucleic acid into the cell, e.g, an RNAi, siRNA, shRNA, endogenous microRNA and/or artificial microRNA. The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target. An inhibitory nucleic acid mediates the targeted cleavage of a target RNA transcript, e.g., via an RNA-induced silencing complex (RISC) pathway, thereby inhibiting the expression and/or activity of the target, e.g, deactivating the target gene.

As described elsewhere herein, wheat has a hexaploid genome. Accordingly, in some embodiments, more than one copy of an inhibitory nucleic acid can be necessary in order to inhibit target gene(s) expression sufficiently to cause a male-sterile phenotype. In some embodiments of any of the aspects, a deactivating modification can comprise 1 or more copies of nucleic acid encoding an inhibitory nucleic acid. In some embodiments of any of the aspects, a deactivating modification can comprise 2 or more copies of nucleic acid encoding an inhibitory nucleic acid. In some embodiments of any of the aspects, a deactivating modification can comprise 3 or more copies of nucleic acid encoding an inhibitory nucleic acid. In some embodiments of any of the aspects, a deactivating modification can comprise 4 or more copies of nucleic acid encoding an inhibitory nucleic acid. In some embodiments of any of the aspects, a deactivating modification can comprise 5 or more copies of nucleic acid encoding an inhibitory nucleic acid. Multiple copies of a nucleic acid encoding an inhibitory nucleic acid can be integrated into the genome at the same loci (e.g., in series), or different loci.

In some embodiment of any of the aspects, the inhibitory nucleic acid can comprise SEQ ID NO: 19. In some embodiment of any of the aspects, the inhibitory nucleic acid can comprise a sequence with at least 90% identity, at least 95% identity, or at least 98% identity with SEQ ID NO: 19. In some embodiment of any of the aspects, the inhibitory nucleic acid can comprise a hairpin molecule comprising SEQ ID NO: 19 and the reverse complement of SEQ ID NO: 19. In some embodiment of any of the aspects, the inhibitory nucleic acid can comprise a sequence with at least 90% identity, at least 95% identity, or at least 98% identity with SEQ ID NO: 19 and a sequence with at least 90% identity, at least 95% identity, or at least 98% identity with the reverse complement of SEQ ID NO: 19.

Alternatively an Mfw and/or Mpew gene may be inhibited by gene-editing so that it no longer fulfils its function (‘gene knockout’). A variety of general methods is known for gene editing. Such editing may involve additions to or deletions from the gene coding sequence or from control (regulatory) sequences upstream or downstream of the coding sequence, but in any case is such as to inhibit production of functional RNA transcript. For example, a gene might be knocked out by inserting one or more additional base pairs of DNA resulting in coding for one or more unsuitable amino-acids, or by creating a premature stop codon so as to substantially shorten the resulting RNA transcript. In a preferred mode of our invention, gene editing comprises only deletion of DNA base sequence. Such editing by deletion, because it contains no additional or heterogenous DNA, is often regarded as environmentally safer and so may require less extensive, and hence less expensive and time-consuming, regulation.

Accordingly, in some embodiments of any of the aspects, a deactivating modification can be a modification that interrupts and/or alters the wild-type coding sequence of the gene, e.g., by deletions which generate a stop codon, transposon, deletion, or frameshift in the coding sequence of the gene.

Several methods of gene-editing are known. Such editing may be done using by various methods, including site-directed mutagenesis employing site-specific nucleases, for example transcription activator-like effector nucleases (TALENs), oligonucleotides, meganucleases, and zinc-finger nucleases. Toolkits and services for zinc-finger nuclease mutagenesis are commercially available, for example EXZACT™ Precision Technology, marketed by Dow AgroSciences.

Particularly preferred methods for gene-editing are the recently-discovered CRISPR-associated (Cas) systems such as CRISPR-Cas9. CRISPR is an acronym for clustered regularly interspaced short palindromic repeats). CRISPR-Cas technology for editing of plant genomes is fully described in Belhaj et al. (2015). This is a practicable, convenient and flexible method of gene editing. It has been shown to work well in plants, see for example in Belhaj et al. (2015) and Shan et al. (2014). The latter paper gives full protocols to enable the system to be applied to modify plant genomes (including wheat) as desired.

As described herein, a deactivating modification can be introduced by utilizing the CRISPR/Cas system. In some embodiments of any of the aspects, a plant or seed with a deactivated Mfw and/or Mpew gene can further comprise an exogenous or introduced endonuclease or a nucleic acid encoding such an endonuclease (e.g., Cas9, a Cas9-derived nickase, or a Cas9 homolog (e.g., Cpf1)). In some embodiments of any of the aspects, a plant or seed with a deactivated Mfw and/or Mpew gene can further comprise a CRISPR RNA sequence designed to target an endonuclease to the gene, e.g. (a crRNA and trans-activating crRNA (tracrRNA) and/or a guide RNA (sgRNA)). Briefly, in order for a Cas9 nuclease (or related nuclease) to recognize and cleave a target nucleic acid molecule, a CRISPR RNA (crRNA) and trans-activating crRNA (tracrRNA) must be present. crRNAs hybridize with tracrRNA to form a guide RNA (sgRNA) which then associates with the Cas9 nuclease. Alternatively, the sgRNA can be provided as a single contiguous sgRNA. Once the sgRNA is complexed with Cas9, the complex can bind to a target nucleic acid molecule. The sgRNA binds specifically to a complementary target sequence via a target-specific sequence in the crRNA portion (e.g., the spacer sequence), while Cas9 itself binds to a protospacer adjacent motif (CRISPR/Cas protospacer-adjacent motif; PAM). The Cas9 nuclease then mediates cleavage of the target nucleic acid to create a double-stranded break within the sequence bound by the sgRNA. In some embodiments of any of the aspects, the sgRNA is provided as a single continuous nucleic acid molecule. In some embodiments of any of the aspects, the sgRNA is provided as a set of hybridized molecules, e.g., a crRNA and tracrRNA. In some embodiments of any of the aspects, the sgRNA is provided as a DNA molecule encoding a sgRNA and/or a crRNA and tracrRNA. Design of sgRNAs, crRNAs, and tracrRNAs are known in the art and described elsewhere herein. Exemplary sgRNA sequences for Mfw1, Mfw2, Mfw3, and Mfw5 are provided elsewhere herein.

In alternative embodiments, a deactivating modification can be introduced by utilizing TALENs or ZFN technology, which are known in the art. Methods of engineering nucleases to achieve a desired sequence specificity are known in the art and are described, e.g., in Kim (2014); Kim (2012); Belhaj et al. (2013); Urnov et al. (2010); Bogdanove et al. (2011); Jinek et al. (2012) Silva et al. (2011); Ran et al. (2013); Carlson et al. (2012); Guerts et al. (2009); Taksu et al. (2010); and Watanabe et al. (2012); each of which is incorporated by reference herein in its entirety.

In embodiments where multiple genes are to be deactivated, e.g., multiple members of a gene family, deactivating modifications can be targeted to shared sequences to minimize the number of modifications and/or individual reagents. Alternatively, deactivating modifications can be targeted to areas that are unique to each gene and a multiplexed approach can be taken. By way of non-limiting example, a gene family can be deactivated utilizing a single CRISPR sgRNA (or equivalent) if the sgRNA is targeted to a sequence found in all members of the gene family; or the gene family can be deactivated utilizing multiple CRISPR sgRNAs (or equivalents) if the sgRNAs are each targeted to sequences not found in each member of the gene family.

In some embodiments of any of the aspects, deactivating modifications can be introduced by means of a mutagen, e.g., ethyl methane sulphonate (EMS), radiation, UV light, aflatoxin B 1, nitrosoguanidine (NG), formaldehyde, acetaldehyde, diepoxyoctane (DEO), depoxybutane (DEB), diethyl sulphate (DES), methylnitrontrosoguanidine (NTG), N-ethyl-N-nitrosourea (ENU), and trimethylpsoralen (TMP). In some embodiments of any of the aspects, deactivating modifications can be introduced, selected, and/or identified by means of TILLING (Targeted Induced Local Lesions IN Genomes) which uses mutagens to generate mutations. TILLING is described in detail, e.g., in Kurowska et al. J Appl Genet 2011 52:371-390 and McCallum et al. Plant Physiol 2000 123:439-442, which are incorporated by reference herein in their entireties.

In some embodiments of any of the aspects, deactivating modifications can be introduced by non-transgenic mutagenesis, e.g., by a method which causes mutations of the nucleic acid sequences of the wheat genome without introducing foreign and/or exogenous nucleic acid molecules into the wheat cell. In some embodiments, non-transgenic mutagenesis can comprise insertions and/or deletions due to mutagenic activity, e.g., indels arising from damage and/or repair processes in the cell. Non-transgenic mutagenesis can utilize, e.g., chemical mutagens (e.g., mutagens not comprising a nucleic acid sequence) and/or radiation sources (e.g., UV light). Non-transgenic mutagenesis excludes the use of, e.g., transposon insertions and/or RNAi. In some embodiments of any of the aspects, non-transgenic mutagenesis does not comprise the use of a site-specific nuclease, e.g., CRISPR-Cas. In some embodiments of any of the aspects, non-transgenic mutagenesis can be used in, e.g., TILLING approaches to generate and/or identify deactivating modifications.

In some embodiments of any of the aspects, the deactivating modification is not a naturally occurring modification, mutation, and/or allele.

In order for a gene to be deactivated, it is necessary to reduce the expression from multiple alleles or copies, e.g., wheat is a hexaploid genome and it may be necessary to reduce expression from all six copies of a given gene. Accordingly, in some embodiments of any of the aspects, a deactivating modification is present at all six copies of a given deactivated gene. The individual deactivating modifications can be identical or they can vary.

In some embodiments of any of the aspects, the deactivation of a first gene can further comprise deactivation of one or more further related genes which display functional redundancy with the first gene. In some embodiments, a plant or cell in which a given gene is deactivated can comprise deactivating modification(s) that deactivate all members of that gene's family. In some embodiments, a plant or cell in which a given gene is deactivated can comprise deactivating modification(s) that deactivate all genes with at least 30% sequence identity at the amino acid level to the gene. In some embodiments, a plant or cell in which a given gene is deactivated can comprise deactivating modification(s) that deactivate all genes with at least 40% sequence identity at the amino acid level to the gene. In some embodiments, a plant or cell in which a given gene is deactivated can comprise deactivating modification(s) that deactivate all genes with at least 50% sequence identity at the amino acid level to the gene. In some embodiments, a plant or cell in which a given gene is deactivated can comprise deactivating modification(s) that deactivate all genes with at least 60% sequence identity at the amino acid level to the gene. In some embodiments, a plant or cell in which a given gene is deactivated can comprise deactivating modification(s) that deactivate all genes with at least 70% sequence identity at the amino acid level to the gene. In some embodiments, a plant or cell in which a given gene is deactivated can comprise deactivating modification(s) that deactivate all genes with at least 80% sequence identity at the amino acid level to the gene. In some embodiments, a plant or cell in which a given gene is deactivated can comprise deactivating modification(s) that deactivate all genes with at least 90% sequence identity at the amino acid level to the gene. In some embodiments, a plant or cell in which a given gene is deactivated can comprise deactivating modification(s) that deactivate all genes with at least 30% sequence identity at the nucleotide level to the gene. In some embodiments, a plant or cell in which a given gene is deactivated can comprise deactivating modification(s) that deactivate all genes with at least 40% sequence identity at the nucleotide level to the gene. In some embodiments, a plant or cell in which a given gene is deactivated can comprise deactivating modification(s) that deactivate all genes with at least 50% sequence identity at the nucleotide level to the gene. In some embodiments, a plant or cell in which a given gene is deactivated can comprise deactivating modification(s) that deactivate all genes with at least 60% sequence identity at the nucleotide level to the gene. In some embodiments, a plant or cell in which a given gene is deactivated can comprise deactivating modification(s) that deactivate all genes with at least 70% sequence identity at the nucleotide level to the gene. In some embodiments, a plant or cell in which a given gene is deactivated can comprise deactivating modification(s) that deactivate all genes with at least 80% sequence identity at the nucleotide level to the gene. In some embodiments, a plant or cell in which a given gene is deactivated can comprise deactivating modification(s) that deactivate all genes with at least 90% sequence identity at the nucleotide level to the gene.

It is contemplated herein that such further related gene(s) can be deactivated by the same type of modification (e.g., the first gene is deactivated by modifying the gene with CRISPR/Cas and the further related gene(s) are deactivated by modifying the further related genes(s) with CRISPR/Cas); with the same modification step (e.g., the first gene is deactivated by modifying the gene with CRISPR/Cas and the further related gene(s) are simultaneously deactivated by modifying the further related genes(s) with the same CRISPR/Cas array, wherein the array targets sequences shared between the first and further genes); or by separate types of modifications (e.g., the first gene is deactivated by modifying the gene with CRISPR/Cas and the further related gene(s) are deactivated by introducing an RNAi construct that targets the further related genes).

Producing male-sterile plants according to the invention may be carried out as follows. Transgenic technology is used to deactivate one or more Mfw genes, for example the Mfw1, Mfw2, Mfw3 and/or Mfw5 genes. Transformation vectors are designed to repress expression of the gene using gene silencing technology. In one application, an RNAi construct is designed and used to produce a quantitative effect on expression of at least one Mfw gene, for example Mfw1. A range of different sterility phenotypes may be produced in this way for assessment. In a second application, a synthetic micro RNA construct is designed and used to achieve complete suppression of an Mfw gene, for example Mfw1. In both applications, Agrobacterium transfer may be used to introduce the constructs into wheat immature embryo cells from which whole wheat plants are derived, for example using known well-established selection and regeneration protocols (e.g., those given in Risacher et al., (2009)).

In one aspect, described herein is a wheat plant or seed that is male-sterile as a result of deactivation of one or more Mfw genes. In one aspect, described herein is a wheat plant or seed that is male-sterile as a result of deactivation of one or more Mpew genes.

In one aspect, described herein is a wheat plant or seed that is male-sterile and comprises a deactivating modification of one or more Mfw genes. In one aspect, described herein is a wheat plant or seed that is male-sterile and comprises a deactivating modification of one or more Mpew genes. In one aspect, described herein is a wheat plant or seed that is male-sterile and comprises a deactivating modification at each copy of one or more Mfw genes. In one aspect, described herein is a wheat plant or seed that is male-sterile and comprises a deactivating modification at each copy of one or more Mpew genes. In one aspect, described herein is a hybrid wheat plant and/or seed comprising at least one copy of a Mfw gene comprising a deactivating modification and at least one wild-type copy of the same Mfw gene. In one aspect, described herein is a hybrid wheat plant and/or seed comprising at least one copy of a Mpew gene comprising a deactivating modification and at least one wild-type copy of the same Mpew gene. In one aspect, described herein is a hybrid wheat plant and/or seed comprising at least three copies of a Mfw gene comprising a deactivating modification and three wild-type copies of the same Mfw gene. In one aspect, described herein is a hybrid wheat plant and/or seed comprising at least three copies of a Mpew gene comprising a deactivating modification and three wild-type copies of the same Mpew gene. In one aspect, described herein is a hybrid wheat plant and/or seed comprising at three copies of a Mfw gene comprising a deactivating modification and three wild-type copies of the same Mfw gene. In one aspect, described herein is a hybrid wheat plant and/or seed comprising three copies of a Mpew gene comprising a deactivating modification and three wild-type copies of the same Mpew gene.

In one aspect of any of the embodiments, described herein is a population of hybrid wheat plants comprising at least one copy of a Mfw gene comprising a deactivating modification and at least one wild-type copy of the same Mfw gene. In one aspect of any of the embodiments, described herein is a population of hybrid wheat plants comprising at least one copy of a Mpew gene comprising a deactivating modification and at least one wild-type copy of the same Mpew gene.

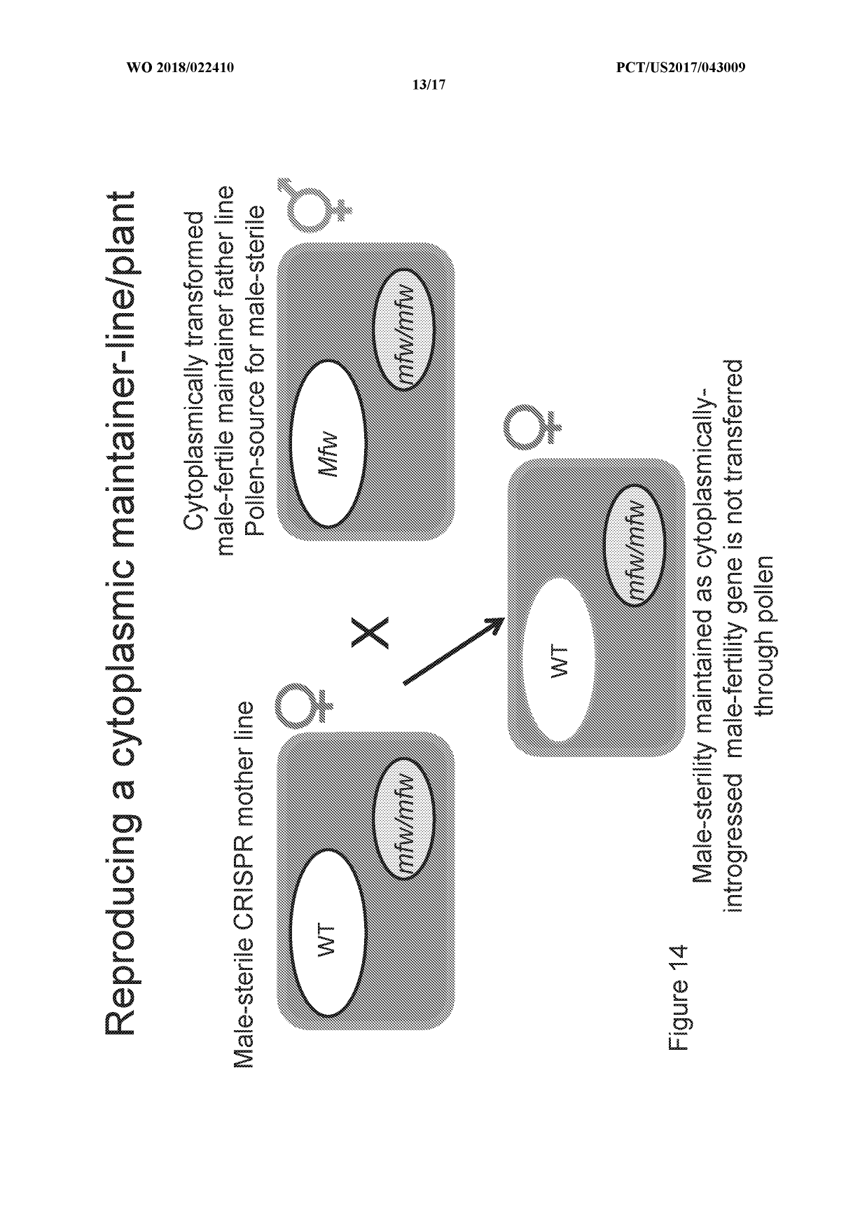

FIG. 15 depicts an illustrative example of the breeding of hybrid plants as described herein. The male sterile plants described herein can be crossed with standard wheat lines which are wild type and dominant for the Mfw and/or Mpew genes. The offspring will be F1 hybrid lines which are male-fertile.

The invention will now be further described with reference to the drawings and the accompanying SEQ IDs NOs 1-19, wherein

SEQ ID NO 1 is the amino-acid sequence for which Mfw1-A codes

SEQ ID NO 2 is the amino-acid sequence for which Mfw1-B codes

SEQ ID NO 3 is the amino-acid sequence for which Mfw1-D codes

SEQ ID NO 4 is the amino-acid sequence for which Mfw2-A codes

SEQ ID NO 5 is the amino-acid sequence for which Mfw2-B codes

SEQ ID NO 6 is the amino-acid sequence for which Mfw2-D codes

SEQ ID NO 7 is the DNA coding sequence (from start codon to stop codon inclusive) of Mfw1-A from wheat (Triticum aestivum, variety ‘Fielder’)

SEQ ID NO 8 is the DNA coding sequence (from start codon to stop codon inclusive) of Mfw1-B from wheat (Triticum aestivum, variety ‘Fielder’)

SEQ ID NO 9 is the DNA coding sequence (from start codon to stop codon inclusive) of Mfw1-D from wheat (Triticum aestivum, variety ‘Fielder’)

SEQ ID NO 10 is the DNA coding sequence (from start codon to stop codon inclusive) of Mfw2-A from wheat (Triticum aestivum, variety ‘Fielder’)

SEQ ID NO 11 is the DNA coding sequence (from start codon to stop codon inclusive) of Mfw2-B from wheat (Triticum aestivum, variety ‘Fielder’)

SEQ ID NO 12 is the DNA coding sequence (from start codon to stop codon inclusive) of Mfw2-D from wheat (Triticum aestivum, variety ‘Fielder’)

SEQ ID NO 13 is a partial sequence of chromosome 7A of wheat (Triticum aestivum, variety ‘Chinese Spring’) including Mfw1-A

SEQ ID NO 14 is a partial sequence chromosome 7A of wheat (Triticum aestivum, variety ‘Chinese Spring’) including Mfw2-A

SEQ ID NO 15 is a partial sequence of chromosome 7B of wheat (Triticum aestivum, variety ‘Chinese Spring’) including Mfw1-B

SEQ ID NO 16 is a partial sequence of chromosome 7B of wheat (Triticum aestivum, variety ‘Chinese Spring’) including Mfw2-B

SEQ ID NO 17 is a partial sequence of chromosome 7D of wheat (Triticum aestivum, variety ‘Chinese Spring’) including Mfw1-D

SEQ ID NO 18 is a partial sequence of chromosome 7D of wheat (Triticum aestivum, variety ‘Chinese Spring’) including Mfw2-D

SEQ ID NO 19 is the DNA sequence to be inserted in Example 2 below.

SEQ ID NO 30 is the amino-acid sequence for which Mfw3-A codes.

SEQ ID NO 31 is the amino-acid sequence for which Mfw3-B codes.

SEQ ID NO 32 is the amino-acid sequence for which Mfw3-D codes.

SEQ ID NO 33 is the amino-acid sequence for which Mfw5-A codes.

SEQ ID NO 34 is the amino-acid sequence for which Mfw5-B codes.

SEQ ID NO 35 is the amino-acid sequence for which Mfw5-D codes.

SEQ ID NO 36 is the DNA coding sequence (from start-codon to stop-codon inclusive) of Mfw3-A from wheat (Triticum aestivum, variety ‘Fielder’).

SEQ ID NO 37 is the DNA coding sequence (from start-codon to stop-codon inclusive) of Mfw3-B from wheat (Triticum aestivum, variety ‘Fielder’).

SEQ ID NO 38 is the DNA coding sequence (from start-codon to stop-codon inclusive) of Mfw3-D from wheat (Triticum aestivum, variety ‘Fielder’).

SEQ ID NO 39 is the DNA coding sequence (from start-codon to stop-codon inclusive) of Mfw5-A from wheat (Triticum aestivum, variety ‘Fielder’).

SEQ ID NO 40 is the DNA coding sequence (from start-codon to stop-codon inclusive) of Mfw5-B from wheat (Triticum aestivum, variety ‘Fielder’).

SEQ ID NO 41 is the DNA coding sequence (from start-codon to stop-codon inclusive) of Mfw5-D from wheat (Triticum aestivum, variety ‘Fielder’).

SEQ ID NO 42 is a partial sequence of chromosome 6A of wheat (Triticum aestivum, variety ‘Chinese Spring’) including Mfw3-A.

SEQ ID NO 43 is a partial sequence of chromosome 6B of wheat (Triticum aestivum, variety ‘Chinese Spring’) including Mfw3-B.

SEQ ID NO 44 is a partial sequence of chromosome 6D of wheat (Triticum aestivum, variety ‘Chinese Spring’) including Mfw3-D.

SEQ ID NO 45 is a partial sequence of chromosome 2A of wheat (Triticum aestivum, variety ‘Chinese Spring’) including Mfw5-A.

SEQ ID NO 46 is a partial sequence of chromosome 2B of wheat (Triticum aestivum, variety ‘Chinese Spring’) including Mfw5-B.

SEQ ID NO 47 is a partial sequence of chromosome 2D of wheat (Triticum aestivum, variety ‘Chinese Spring’) including Mfw5-D.

SEQ ID NO 48 is the DNA sequence to be inserted in Example 6.

SEQ ID NO 60 is the amino-acid sequence for which Mfw4-A codes.

SEQ ID NO 61 is the DNA coding sequence (from start-codon to stop-codon inclusive) of Mfw4-A from wheat (Triticum aestivum, variety ‘Fielder’).

SEQ ID NO 62 is a partial sequence of the wheat (Triticum aestivum, variety ‘Chinese Spring’) genomic sequence including Mfw4-A.

SEQ ID NO 63 is the amino-acid sequence for which Mfw4-B codes.

SEQ ID NO 64 is the DNA coding sequence (from start-codon to stop-codon inclusive) of Mfw4-B from wheat (Triticum aestivum, variety ‘Fielder’).

SEQ ID NO 65 is a partial sequence of the wheat (Triticum aestivum, variety ‘Chinese Spring’) genomic sequence including Mfw4-B.

SEQ ID NO 66 is the amino-acid sequence for which Mfw4-D codes.

SEQ ID NO 67 is the DNA coding sequence (from start-codon to stop-codon inclusive) of Mfw4-D from wheat (Triticum aestivum, variety ‘Fielder’).

SEQ ID NO 68 is a partial sequence of the wheat (Triticum aestivum, variety ‘Chinese Spring’) genomic sequence including Mfw4-D.

SEQ ID NO 69 is the amino-acid sequence for which Mfw6-A codes.

SEQ ID NO 70 is the DNA coding sequence (from start-codon to stop-codon inclusive) of Mfw6-A from wheat (Triticum aestivum, variety ‘Fielder’).

SEQ ID NO 71 is a partial sequence of the wheat (Triticum aestivum, variety ‘Chinese Spring’) genomic sequence including Mfw6-A.

SEQ ID NO 72 is the amino-acid sequence for which Mfw6-D codes.

SEQ ID NO 73 is the DNA coding sequence (from start-codon to stop-codon inclusive) of Mfw6-D from wheat (Triticum aestivum, variety ‘Fielder’).

SEQ ID NO 74 is a partial sequence of the wheat (Triticum aestivum, variety ‘Chinese Spring’) genomic sequence including Mfw6-D.

SEQ ID NO 75 is the amino-acid sequence for which Mfw7-A codes.

SEQ ID NO 76 is the DNA coding sequence (from start-codon to stop-codon inclusive) of Mfw7-A from wheat (Triticum aestivum, variety ‘Fielder’).

SEQ ID NO 77 is a partial sequence of the wheat (Triticum aestivum, variety ‘Chinese Spring’) genomic sequence including Mfw7-A.

SEQ ID NO 78 is the amino-acid sequence for which Mfw7-B codes.

SEQ ID NO 79 is the DNA coding sequence (from start-codon to stop-codon inclusive) of Mfw7-B from wheat (Triticum aestivum, variety ‘Fielder’).

SEQ ID NO 80 is a partial sequence of the wheat (Triticum aestivum, variety ‘Chinese Spring’) genomic sequence including Mfw7-B.

SEQ ID NO 81 is the amino-acid sequence for which Mfw7-D codes.

SEQ ID NO 82 is the DNA coding sequence (from start-codon to stop-codon inclusive) of Mfw7-D from wheat (Triticum aestivum, variety ‘Fielder’).

SEQ ID NO 83 is a partial sequence of the wheat (Triticum aestivum, variety ‘Chinese Spring’) genomic sequence including Mfw7-D.

SEQ ID NO 84 is the amino-acid sequence for which Mfw8-A codes.

SEQ ID NO 85 is the DNA coding sequence (from start-codon to stop-codon inclusive) of Mfw8-A from wheat (Triticum aestivum, variety ‘Fielder’).

SEQ ID NO 86 is a partial sequence of the wheat (Triticum aestivum, variety ‘Chinese Spring’) genomic sequence including Mfw8-A.

SEQ ID NO 87 is the amino-acid sequence for which Mfw8-B codes.

SEQ ID NO 88 is the DNA coding sequence (from start-codon to stop-codon inclusive) of Mfw8-B from wheat (Triticum aestivum, variety ‘Fielder’).

SEQ ID NO 89 is a partial sequence of the wheat (Triticum aestivum, variety ‘Chinese Spring’) genomic sequence including Mfw8-B.

SEQ ID NO 90 is the amino-acid sequence for which Mfw8-D codes.

SEQ ID NO 91 is the DNA coding sequence (from start-codon to stop-codon inclusive) of Mfw8-D from wheat (Triticum aestivum, variety ‘Fielder’).

SEQ ID NO 92 is a partial sequence of the wheat (Triticum aestivum, variety ‘Chinese Spring’) genomic sequence including Mfw8-D.

SEQ ID NO 93 is the amino-acid sequence for which Mfw9-A codes.

SEQ ID NO 94 is the DNA coding sequence (from start-codon to stop-codon inclusive) of Mfw9-A from wheat (Triticum aestivum, variety ‘Fielder’).

SEQ ID NO 95 is a partial sequence of the wheat (Triticum aestivum, variety ‘Chinese Spring’) genomic sequence including Mfw9-A.

SEQ ID NO 96 is the amino-acid sequence for which Mfw9-B codes.

SEQ ID NO 97 is the DNA coding sequence (from start-codon to stop-codon inclusive) of Mfw9-B from wheat (Triticum aestivum, variety ‘Fielder’).

SEQ ID NO 98 is a partial sequence of the wheat (Triticum aestivum, variety ‘Chinese Spring’) genomic sequence including Mfw9-B.

SEQ ID NO 99 is the amino-acid sequence for which Mfw9-D codes.

SEQ ID NO 100 is the DNA coding sequence (from start-codon to stop-codon inclusive) of Mfw9-D from wheat (Triticum aestivum, variety ‘Fielder’).

SEQ ID NO 101 is a partial sequence of the wheat (Triticum aestivum, variety ‘Chinese Spring’) genomic sequence including Mfw9-D.

SEQ ID NO 102 is the amino-acid sequence for which Mfw10-A codes.

SEQ ID NO 103 is the DNA coding sequence (from start-codon to stop-codon inclusive) of Mfw10-A from wheat (Triticum aestivum, variety ‘Fielder’).

SEQ ID NO 104 is a partial sequence of the wheat (Triticum aestivum, variety ‘Chinese Spring’) genomic sequence including Mfw10-A.

SEQ ID NO 105 is the amino-acid sequence for which Mfw10-B codes.

SEQ ID NO 106 is the DNA coding sequence (from start-codon to stop-codon inclusive) of Mfw10-B from wheat (Triticum aestivum, variety ‘Fielder’).

SEQ ID NO 107 is a partial sequence of the wheat (Triticum aestivum, variety ‘Chinese Spring’) genomic sequence including Mfw11-U.

SEQ ID NO 108 is the amino-acid sequence for which Mfw11-U codes.

SEQ ID NO 109 is the DNA coding sequence (from start-codon to stop-codon inclusive) of Mfw11-U from wheat (Triticum aestivum, variety ‘Fielder’).

SEQ ID NO 110 is a partial sequence of the wheat (Triticum aestivum, variety ‘Chinese Spring’) genomic sequence including Mfw11-U.

SEQ ID NO 111 is the amino-acid sequence for which Mfw12-A codes.

SEQ ID NO 112 is the DNA coding sequence (from start-codon to stop-codon inclusive) of Mfw12-A from wheat (Triticum aestivum, variety ‘Fielder’).

SEQ ID NO 113 is a partial sequence of the wheat (Triticum aestivum, variety ‘Chinese Spring’) genomic sequence including Mfw12-A.

SEQ ID NO 114 is the amino-acid sequence for which Mfw12-B codes.

SEQ ID NO 115 is the DNA coding sequence (from start-codon to stop-codon inclusive) of Mfw12-B from wheat (Triticum aestivum, variety ‘Fielder’).

SEQ ID NO 116 is a partial sequence of the wheat (Triticum aestivum, variety ‘Chinese Spring’) genomic sequence including Mfw12-B.

SEQ ID NO 117 is the amino-acid sequence for which Mfw12-D codes.

SEQ ID NO 118 is the DNA coding sequence (from start-codon to stop-codon inclusive) of Mfw12-D from wheat (Triticum aestivum, variety ‘Fielder’).

SEQ ID NO 119 is a partial sequence of the wheat (Triticum aestivum, variety ‘Chinese Spring’) genomic sequence including Mfw12-D.

SEQ ID NO 120 is the amino-acid sequence for which Mfw13-A codes.

SEQ ID NO 121 is the DNA coding sequence (from start-codon to stop-codon inclusive) of Mfw13-A from wheat (Triticum aestivum, variety ‘Fielder’).

SEQ ID NO 122 is a partial sequence of the wheat (Triticum aestivum, variety ‘Chinese Spring’) genomic sequence including Mfw13-A.

SEQ ID NO 123 is the amino-acid sequence for which Mfw13-B codes.

SEQ ID NO 124 is the DNA coding sequence (from start-codon to stop-codon inclusive) of Mfw13-B from wheat (Triticum aestivum, variety ‘Fielder’).

SEQ ID NO 125 is a partial sequence of the wheat (Triticum aestivum, variety ‘Chinese Spring’) genomic sequence including Mfw13-D.

SEQ ID NO 126 is the amino-acid sequence for which Mfw13-B codes.

SEQ ID NO 127 is the DNA coding sequence (from start-codon to stop-codon inclusive) of Mfw13-D from wheat (Triticum aestivum, variety ‘Fielder’).

SEQ ID NO 128 is a partial sequence of the wheat (Triticum aestivum, variety ‘Chinese Spring’) genomic sequence including Mfw13-D.

All samples of genetic resources used in the Examples were obtained in the UK, from stock reproduced in the UK. The wheat variety ‘Fielder’ was originally bred in the USA.

Further description of SEQ ID NOs 13-18

SEQ ID NO 13 is a partial sequence of that part of chromosome 7A of wheat (Triticum aestivum, variety ‘Chinese Spring’) that, from the start codon starting at 6072 bp to the end of the TAA stop codon at 8122 bp, includes the DNA coding sequence for Mfw1-A as well as flanking sequences upstream of the start codon and downstream of the stop codon. These flanking sequences may be expected to include regulatory sequences, such as, in the upstream flanking sequence, the promoter.

SEQ ID NO 14 is a partial sequence of that part of chromosome 7B of wheat (Triticum aestivum, variety ‘Chinese Spring’) that, from the start codon starting at 2076 bp to the end of the TAA stop codon at 3844 bp, includes the DNA coding sequence for Mfw2-A as well as flanking sequences upstream of the start codon and downstream of the stop codon. These flanking sequences may be expected to include regulatory sequences, such as, in the upstream flanking sequence, the promoter.

SEQ ID NO 15 is a partial sequence of that part of chromosome 7D of wheat (Triticum aestivum, variety ‘Chinese Spring’) that, from the start codon starting at 7957 bp to the end of the TAA stop codon at 9960 bp, includes the DNA coding sequence for Mfw1-B as well as flanking sequences upstream of the start codon and downstream of the stop codon. These flanking sequences may be expected to include regulatory sequences, such as, in the upstream flanking sequence, the promoter.

SEQ ID NO 16 is a partial sequence of that part of chromosome 7A of wheat (Triticum aestivum, variety ‘Chinese Spring’) that, from the start codon starting at 2949 bp to the end of the TGA stop codon at 16953 bp, includes the DNA coding sequence for Mfw2-B as well as flanking sequences upstream of the start codon and downstream of the stop codon. These flanking sequences may be expected to include regulatory sequences, such as, in the upstream flanking sequence, the promoter.

SEQ ID NO 17 is a partial sequence of that part of chromosome 7B of wheat (Triticum aestivum, variety ‘Chinese Spring’) that, from the start codon starting at 249 bp to the end of the TGA stop codon at 17681 bp, includes the DNA coding sequence for Mfw1-D as well as flanking sequences upstream of the start codon and downstream of the stop codon. These flanking sequences may be expected to include regulatory sequences, such as, in the upstream flanking sequence, the promoter.

SEQ ID NO 18 is a partial sequence of that part of chromosome 7D of wheat (Triticum aestivum, variety ‘Chinese Spring’) that, from the start codon starting at 1255 bp to the end of the TGA stop codon at 18448 bp, includes the DNA coding sequence for Mfw2-D as well as flanking sequences upstream of the start codon and downstream of the stop codon. These flanking sequences may be expected to include regulatory sequences, such as, in the upstream flanking sequence, the promoter.

SEQ ID Nos 13-18 are taken from the public literature referred to above.

Further description of SEQ ID NOs

SEQ ID NO 42 is a partial sequence of that part of chromosome 6A of wheat (Triticum aestivum, variety ‘Chinese Spring’) that, from the start codon starting at 2130 bp to the end of the TGA stop codon at 4398 bp, includes the DNA coding sequence for Mfw3-A as well as flanking sequences upstream of the start codon and downstream of the stop codon. These flanking sequences may be expected to include regulatory sequences, such as, in the upstream flanking sequence, the promoter.

SEQ ID NO 43 is a partial sequence of that part of chromosome 6B of wheat (Triticum aestivum, variety ‘Chinese Spring’) that, from the start codon starting at 1884 bp to the end of the TGA stop codon at 4144 bp, includes the DNA coding sequence for Mfw3-B as well as flanking sequences upstream of the start codon and downstream of the stop codon. These flanking sequences may be expected to include regulatory sequences, such as, in the upstream flanking sequence, the promoter.

SEQ ID NO 44 is a partial sequence of that part of chromosome 6D of wheat (Triticum aestivum, variety ‘Chinese Spring’) that, from the start codon starting at 2078 bp to the end of the TGA stop codon at 4269 bp, includes the DNA coding sequence for Mfw3-D as well as flanking sequences upstream of the start codon and downstream of the stop codon. These flanking sequences may be expected to include regulatory sequences, such as, in the upstream flanking sequence, the promoter.

SEQ ID NO 45 is a partial sequence of that part of chromosome 2A of wheat (Triticum aestivum, variety ‘Chinese Spring’) that, from the start codon starting at 1395 bp to the end of the TGA stop codon at 3650 bp, includes the DNA coding sequence for Mfw5-A as well as flanking sequences upstream of the start codon and downstream of the stop codon. These flanking sequences may be expected to include regulatory sequences, such as, in the upstream flanking sequence, the promoter.

SEQ ID NO 46 is a partial sequence of that part of chromosome 2B of wheat (Triticum aestivum, variety ‘Chinese Spring’) that, from the start codon starting at 2360 bp to the end of the TGA stop codon at 4734 bp, includes the DNA coding sequence for Mfw5-B as well as flanking sequences upstream of the start codon and downstream of the stop codon. These flanking sequences may be expected to include regulatory sequences, such as, in the upstream flanking sequence, the promoter.

SEQ ID NO 47 is a partial sequence of that part of chromosome 2D of wheat (Triticum aestivum, variety ‘Chinese Spring’) that, from the start codon starting at 1501 bp to the end of the TGA stop codon at 3579 bp, includes the DNA coding sequence for Mfw5-D as well as flanking sequences upstream of the start codon and downstream of the stop codon. These flanking sequences may be expected to include regulatory sequences, such as, in the upstream flanking sequence, the promoter.

SEQ ID NO 62 is a partial sequence of that part of the genomic sequence of wheat (Triticum aestivum, variety ‘Chinese Spring’) that, from the start codon starting at 1374 bp to the end of the TGA stop codon at 4938 bp, includes the DNA coding sequence for Mfw4-A as well as flanking sequences upstream of the start codon and downstream of the stop codon. These flanking sequences may be expected to include regulatory sequences, such as, in the upstream flanking sequence, the promoter.

SEQ ID NO 65 is a partial sequence of that part of the genomic sequence of wheat (Triticum aestivum, variety ‘Chinese Spring’) that, from the start codon starting at 1309 bp to the end of the TGA stop codon at 4637 bp, includes the DNA coding sequence for Mfw4-B as well as flanking sequences upstream of the start codon and downstream of the stop codon. These flanking sequences may be expected to include regulatory sequences, such as, in the upstream flanking sequence, the promoter.

SEQ ID NO 68 is a partial sequence of that part of the genomic sequence of wheat (Triticum aestivum, variety ‘Chinese Spring’) that, from the start codon starting at 1309 bp to the end of the TGA stop codon at 4637 bp, includes the DNA coding sequence for Mfw4-D as well as flanking sequences upstream of the start codon and downstream of the stop codon. These flanking sequences may be expected to include regulatory sequences, such as, in the upstream flanking sequence, the promoter.

SEQ ID NO 71 is a partial sequence of that part of the genomic sequence of wheat (Triticum aestivum, variety ‘Chinese Spring’) that, from the start codon starting at 1605 bp to the end of the TGA stop codon at 3022 bp, includes the DNA coding sequence for Mfw6-A as well as flanking sequences upstream of the start codon and downstream of the stop codon. These flanking sequences may be expected to include regulatory sequences, such as, in the upstream flanking sequence, the promoter.

SEQ ID NO 74 is a partial sequence of that part of the genomic sequence of wheat (Triticum aestivum, variety ‘Chinese Spring’) that, from the start codon starting at 1560 bp to the end of the TGA stop codon at 2980 bp, includes the DNA coding sequence for Mfw6-D as well as flanking sequences upstream of the start codon and downstream of the stop codon. These flanking sequences may be expected to include regulatory sequences, such as, in the upstream flanking sequence, the promoter.

SEQ ID NO 77 is a partial sequence of that part of the genomic sequence of wheat (Triticum aestivum, variety ‘Chinese Spring’) that, from the start codon starting at 1318 bp to the end of the TGA stop codon at 3470 bp, includes the DNA coding sequence for Mfw7-A as well as flanking sequences upstream of the start codon and downstream of the stop codon. These flanking sequences may be expected to include regulatory sequences, such as, in the upstream flanking sequence, the promoter.

SEQ ID NO 80 is a partial sequence of that part of the genomic sequence of wheat (Triticum aestivum, variety ‘Chinese Spring’) that, from the start codon starting at 1229 bp to the end of the TGA stop codon at 3369 bp, includes the DNA coding sequence for Mfw7-B as well as flanking sequences upstream of the start codon and downstream of the stop codon. These flanking sequences may be expected to include regulatory sequences, such as, in the upstream flanking sequence, the promoter.

SEQ ID NO 83 is a partial sequence of that part of the genomic sequence of wheat (Triticum aestivum, variety ‘Chinese Spring’) that, from the start codon starting at 1413 bp to the end of the TGA stop codon at 3588 bp, includes the DNA coding sequence for Mfw7-D as well as flanking sequences upstream of the start codon and downstream of the stop codon. These flanking sequences may be expected to include regulatory sequences, such as, in the upstream flanking sequence, the promoter.

SEQ ID NO 86 is a partial sequence of that part of the genomic sequence of wheat (Triticum aestivum, variety ‘Chinese Spring’) that, from the start codon starting at 1340 bp to the end of the TGA stop codon at 3407 bp, includes the DNA coding sequence for Mfw8-A as well as flanking sequences upstream of the start codon and downstream of the stop codon. These flanking sequences may be expected to include regulatory sequences, such as, in the upstream flanking sequence, the promoter.

SEQ ID NO 87 is a partial sequence of that part of the genomic sequence of wheat (Triticum aestivum, variety ‘Chinese Spring’) that, from the start codon starting at 1349 bp to the end of the TGA stop codon at 3422 bp, includes the DNA coding sequence for Mfw8-B as well as flanking sequences upstream of the start codon and downstream of the stop codon. These flanking sequences may be expected to include regulatory sequences, such as, in the upstream flanking sequence, the promoter.

SEQ ID NO 92 is a partial sequence of that part of the genomic sequence of wheat (Triticum aestivum, variety ‘Chinese Spring’) that, from the start codon starting at 1331 bp to the end of the TGA stop codon at 3401 bp, includes the DNA coding sequence for Mfw8-D as well as flanking sequences upstream of the start codon and downstream of the stop codon. These flanking sequences may be expected to include regulatory sequences, such as, in the upstream flanking sequence, the promoter.

SEQ ID NO 95 is a partial sequence of that part of the genomic sequence of wheat (Triticum aestivum, variety ‘Chinese Spring’) that, from the start codon starting at 1248 bp to the end of the TGA stop codon at 2849 bp, includes the DNA coding sequence for Mfw9-A as well as flanking sequences upstream of the start codon and downstream of the stop codon. These flanking sequences may be expected to include regulatory sequences, such as, in the upstream flanking sequence, the promoter.

SEQ ID NO 98 is a partial sequence of that part of the genomic sequence of wheat (Triticum aestivum, variety ‘Chinese Spring’) that, from the start codon starting at 393 bp to the end of the TGA stop codon at 32502 bp, includes the DNA coding sequence for Mfw9-B as well as flanking sequences upstream of the start codon and downstream of the stop codon. These flanking sequences may be expected to include regulatory sequences, such as, in the upstream flanking sequence, the promoter.

SEQ ID NO 101 is a partial sequence of that part of the genomic sequence of wheat (Triticum aestivum, variety ‘Chinese Spring’) that, from the start codon starting at 1273 bp to the end of the TGA stop codon at 2831 bp, includes the DNA coding sequence for Mfw9-D as well as flanking sequences upstream of the start codon and downstream of the stop codon. These flanking sequences may be expected to include regulatory sequences, such as, in the upstream flanking sequence, the promoter.

SEQ ID NO 104 is a partial sequence of that part of the genomic sequence of wheat (Triticum aestivum, variety ‘Chinese Spring’) that, from the start codon starting at 1398 bp to the end of the TGA stop codon at 3217 bp, includes the DNA coding sequence for Mfw10-A as well as flanking sequences upstream of the start codon and downstream of the stop codon. These flanking sequences may be expected to include regulatory sequences, such as, in the upstream flanking sequence, the promoter.

SEQ ID NO 107 is a partial sequence of that part of the genomic sequence of wheat (Triticum aestivum, variety ‘Chinese Spring’) that, from the start codon starting at 1407 bp to the end of the TGA stop codon at 3217 bp, includes the DNA coding sequence for Mfw10-B as well as flanking sequences upstream of the start codon and downstream of the stop codon. These flanking sequences may be expected to include regulatory sequences, such as, in the upstream flanking sequence, the promoter.

SEQ ID NO 110 is a partial sequence of that part of the genomic sequence of wheat (Triticum aestivum, variety ‘Chinese Spring’) that, from the start codon starting at 1553 bp to the end of the TGA stop codon at 2940 bp, includes the DNA coding sequence for Mfw11-U as well as flanking sequences upstream of the start codon and downstream of the stop codon. These flanking sequences may be expected to include regulatory sequences, such as, in the upstream flanking sequence, the promoter.

SEQ ID NO 113 is a partial sequence of that part of the genomic sequence of wheat (Triticum aestivum, variety ‘Chinese Spring’) that, from the start codon starting at 1309 bp to the end of the TGA stop codon at 3246 bp, includes the DNA coding sequence for Mfw12-A as well as flanking sequences upstream of the start codon and downstream of the stop codon. These flanking sequences may be expected to include regulatory sequences, such as, in the upstream flanking sequence, the promoter.

SEQ ID NO 116 is a partial sequence of that part of the genomic sequence of wheat (Triticum aestivum, variety ‘Chinese Spring’) that, from the start codon starting at 1281 bp to the end of the TGA stop codon at 3169 bp, includes the DNA coding sequence for Mfw12-B as well as flanking sequences upstream of the start codon and downstream of the stop codon. These flanking sequences may be expected to include regulatory sequences, such as, in the upstream flanking sequence, the promoter.

SEQ ID NO 119 is a partial sequence of that part of the genomic sequence of wheat (Triticum aestivum, variety ‘Chinese Spring’) that, from the start codon starting at 1300 bp to the end of the TGA stop codon at 3086 bp, includes the DNA coding sequence for Mfw12-D as well as flanking sequences upstream of the start codon and downstream of the stop codon. These flanking sequences may be expected to include regulatory sequences, such as, in the upstream flanking sequence, the promoter.

SEQ ID NO 122 is a partial sequence of that part of the genomic sequence of wheat (Triticum aestivum, variety ‘Chinese Spring’) that, from the start codon starting at 1308 bp to the end of the TGA stop codon at 3251 bp, includes the DNA coding sequence for Mfw13-A as well as flanking sequences upstream of the start codon and downstream of the stop codon. These flanking sequences may be expected to include regulatory sequences, such as, in the upstream flanking sequence, the promoter.

SEQ ID NO 125 is a partial sequence of that part of the genomic sequence of wheat (Triticum aestivum, variety ‘Chinese Spring’) that, from the start codon starting at 1259 bp to the end of the TGA stop codon at 3233 bp, includes the DNA coding sequence for Mfw13-B as well as flanking sequences upstream of the start codon and downstream of the stop codon. These flanking sequences may be expected to include regulatory sequences, such as, in the upstream flanking sequence, the promoter.

SEQ ID NO 128 is a partial sequence of that part of the genomic sequence of wheat (Triticum aestivum, variety ‘Chinese Spring’) that, from the start codon starting at 1446 bp to the end of the TGA stop codon at 3418 bp, includes the DNA coding sequence for Mfw13-D as well as flanking sequences upstream of the start codon and downstream of the stop codon. These flanking sequences may be expected to include regulatory sequences, such as, in the upstream flanking sequence, the promoter.

In some embodiments of any of the aspects, Mfw1, Mfw2, Mfw3, and/or Mfw5 genes can be deactivated in wheat plants by utilizing a CRISPR/Cas system to introduce deactivating mutations at these loci. For example, Mfw1 and Mfw2 genes can be targeted with four guide RNAs for each of the three sets of homoeologues. The target sequences in these genes can be identified using the publicly available program DREG (available on the world wide web at emboss.sourceforge.net/apps/cvs/emboss/apps/dreg.html) to find sequences that match either ANNNNNNNNNNNNNNNNNNNNGG or GNNNNNNNNNNNNNNNNNNNNNGG in both directions of the Fielder genomic sequence.

As an illustrative example, the guides can be selected from the results based on the following criteria: that the target sequence is conserved in all three homoeologues, that it is (at least partially) in an exon of Mfw1 or Mfw2 genes, that it has a restriction enzyme site near the site of the protospacer associated motif (PAM) but in the sequence of the guide RNA and finally, prioritizing guides near the start of the coding sequences of each gene.

An additional consideration can be to select sequences with either AN20GG and GN20GG as this stabilizes the construct for transformation in the plant. Exemplary guide sequences are depicted within the context of SEQ ID NOs 20-21 below and are individually identified, in order, as SEQ ID NOs 22-29. Guide sequence expression can be driven by individual and/or shared promoters. Exemplary promoters include OsU3, TaU3, TaU6 and OsU6 promoters

Guide constructs, expressing one or more sgRNA sequences can be cloned into a vector suitable for expressing the sgRNAs in wheat, e.g., a binary vector containing a wheat-optimized Cas9 enzyme driven by the rice actin promoter. Vectors can be introduced into wheat by any means known in the art, e.g. by Agrobacterium. Alternatively, the sgRNAs can be expressed in vitro and introduced into wheat cells by, e.g., microinjection.

Plants can be screened for deactivating modifications, e.g., utilizing a PCR based method where the PCR product is digested with an appropriate enzyme previously identified to cut the DNA at a site near the PAM. PCR products which are not cut therefore contain a mutation induced by the CRISPR construct.

Sequence for Mfw1 guides (guide targeting sequences shown in
bold)
(SEQ ID NO: 20)
CAAATAATGATTTTATTTTGACTGATAGTGACCTGTTCGTTGCAACAAATTGATG
AGCAATGCTTTTTTATAATGCCAAGTTTGTACAAAAAAGCAGGCTTTAACCGCGG
TATACAAGGAATCTTTAAACATACGAACAGATCACTTAAAGTTCTTCTGAAGCAA
CTTAAAGTTATCAGGCATGCATGGATCTTGGAGGAATCAGATGTGCAGTCAGGG
ACCATAGCACAAGACAGGCGTCTTCTACTGGTGCTACCAGCAAATGCTGGAAGC
CGGGAACACTGGGTACGTTGGAAACCACGTGATGTGAAGAAGTAAGATAAACTG
TAGGAGAAAAGCATTTCGTAGTGGGCCATGAAGCCTTTCAGGACATGTATTGCA
GTATGGGCCGGCCCATTACGCAATTGGACGACAACAAAGACTAGTATTAGTACC
ACCTCGGCTATCCACATAGATCAAAGCTGATTTAAAAGAGTTGTGCAGATGATCC
GTGGCATCGGGAATGTCATCTCCTTGTTTTAGAGCTAGAAATAGCAAGTTAAA
ATAAGGCTAGTCCGTTATCAACTTGAAAAAGTGGCACCGAGTCGGTGCTTTTTTT
TATAACTTAAGCCGCGGGTATACTTAATTAAATTGGATGATCTGATAATTAACCC
GGGGACCAAGCCCGTTATTCTGACAGTTCTGGTGCTCAACACATTTATATTTATC
AAGGAGCACATTGTTACTCACTGCTAGGAGGGAATCGAACTAGGAATATTGATC
AGAGGAACTACGAGAGAGCTGAAGATAACTGCCCTCTAGCTCTCACTGATCTGG
GTCGCATAGTGAGATGCAGCCCACGTGAGTTCAGCAACGGTCTAGCGCTGGGCT
TTTAGGCCCGCATGATCGGGCTTTTGTCGGGTGGTCGACGTGTTCACGATTGGGG
AGAGCAACGCAGCAGTTCCTCTTAGTTTAGTCCCACCTCGCCTGTCCAGCAGAGT
TCTGACCGGTTTATAAACTCGCTTGCTGCATCAGACTTGTACGTACCATGATGG
TGAGGTTTTAGAGCTAGAAATAGCAAGTTAAAATAAGGCTAGTCCGTTATCAAC
TTGAAAAAGTGGCACCGAGTCGGTGCTTTTTTTCCGCGGCACGTCTCGAGCCCGG
GTTAATTAAATTGGATGATGACTCTAGATAACGCAGAAGATTAATTAACCCGGG
GACCAAGCCCGTTATTCTGACAGTTCTGGTGCTCAACACATTTATATTTATCAAG
GAGCACATTGTTACTCACTGCTAGGAGGGAATCGAACTAGGAATATTGATCAGA
GGAACTACGAGAGAGCTGAAGATAACTGCCCTCTAGCTCTCACTGATCTGGGTC
GCATAGTGAGATGCAGCCCACGTGAGTTCAGCAACGGTCTAGCGCTGGGCTTTTA
GGCCCGCATGATCGGGCTTTTGTCGGGTGGTCGACGTGTTCACGATTGGGGAGAG
CAACGCAGCAGTTCCTCTTAGTTTAGTCCCACCTCGCCTGTCCAGCAGAGTTCTG
ACCGGTTTATAAACTCGCTTGCTGCATCAGACTTGATCATCAAGGCCAAGGACG
GTTTTAGAGCTAGAAATAGCAAGTTAAAATAAGGCTAGTCCGTTATCAACTTGAA
AAAGTGGCACCGAGTCGGTGCTTTTTTTCCGCGGCACGTCTCGAGCCCGGGTTAA
TTAAATTGGATGATGACTCTAGATAACGCAGGATCCACTAGTAACGGCCGCCAG
TGTGCTGGAATTGCCCTTGGATCATGAACCAACGGCCTGGCTGTATTTGGTGGTT
GTGTAGGGAGATGGGGAGAAGAAAAGCCCGATTCTCTTCGCTGTGATGGGCTGG
ATGCATGCGGGGGAGCGGGAGGCCCAAGTACGTGCACGGTGAGCGGCCCACAG
GGCGAGTGTGAGCGCGAGAGGCGGGAGGAACAGTTTAGTACCACATTGCCCAGC
TAACTCGAACGCGACCAACTTATAAACCCGCGCGCTGTCGCTTGTGTGGGGGAT
GGGGGCTTACGTAGTTTTAGAGCTAGAAATAGCAAGTTAAAATAAGGCTAGTC
CGTTATCAACTTGAAAAAGTGGCACCGAGTCGGTGCTTTTTTTGTCCCTTCGAAG
GGCAATTCTGCAGATATCCATCACACTGGCGGCCGCTCGAGGTCGAGGGTATCG
ATAAGCTTGAATTCGACCCAGCTTTCTTGTACAAAGTTGGCATTATAAAAAATAA
TTGCTCATCAATTTGTTGCAACGAACAGGTCACTATCAGTCAAAATAAAATCATT
ATTTG
SEQ ID NO: 22
TCGGGAATGTCATCTCCTT
SEQ ID NO: 23
TACGTACCATGATGGTGAG
SEQ ID NO: 24
ATCATCAAGGCCAAGGACG
SEQ ID NO: 25
GGGGATGGGGGCTTACGTA
Sequence for Mfw2 guides (guide targeting sequences shown
in bold)
(SEQ ID NO 21)
CAAATAATGATTTTATTTTGACTGATAGTGACCTGTTCGTTGCAACAAATTGATG
AGCAATGCTTTTTTATAATGCCAAGTTTGTACAAAAAAGCAGGCTTTAACCGCGG
TATACAAGGAATCTTTAAACATACGAACAGATCACTTAAAGTTCTTCTGAAGCAA
CTTAAAGTTATCAGGCATGCATGGATCTTGGAGGAATCAGATGTGCAGTCAGGG
ACCATAGCACAAGACAGGCGTCTTCTACTGGTGCTACCAGCAAATGCTGGAAGC
CGGGAACACTGGGTACGTTGGAAACCACGTGATGTGAAGAAGTAAGATAAACTG
TAGGAGAAAAGCATTTCGTAGTGGGCCATGAAGCCTTTCAGGACATGTATTGCA
GTATGGGCCGGCCCATTACGCAATTGGACGACAACAAAGACTAGTATTAGTACC
ACCTCGGCTATCCACATAGATCAAAGCTGATTTAAAAGAGTTGTGCAGATGATCC
GTGGCACACCTGATTGTTTCTCACTGTTTTAGAGCTAGAAATAGCAAGTTAAAA
TAAGGCTAGTCCGTTATCAACTTGAAAAAGTGGCACCGAGTCGGTGCTTTTTTTT
ATAACTTAAGCCGCGGGTATACTTAATTAAATTGGATGATCTGACTAGATACCGG
TCTCGAGTTAACATGAATCCAAACCACACGGAGTTCAAATTCCCACAGATTAAG
GCTCGTCCGTCGCACAAGGTAATGTGTGAATATTATATCTGTCGTGCAAAATTGC
CTGGCCTGCACAATTGCTGTTATAGTTGGCGGCAGGGAGAGTTTTAACATTGACT
AGCGTGCTGATAATTTGTGAGAAATAATAATTGACAAGTAGATACTGACATTTGA
GAAGAGCTTCTGAACTGTTATTAGTAACAAAAATGGAAAGCTGATGCACGGAAA
AAGGAAAGAAAAAGCCATACTTTTTTTTAGGTAGGAAAAGAAAAAGCCATACGA
GACTGATGTCTCTCAGATGGGCCGGGATCTGTCTATCTAGCAGGCAGCAGCCCTA
CCAACCTCACGGGCCAGCAATTACGAGTCCTTCTAAAACGTCCCGCCGAGGGCG
CGTGGCCGTGCTGTGCAGCAGCACGTCTAACATTAGTCCCACCTCGCCAGTTTAC
AGGGAGCAGAACCAGCTTATAAGCGGAGGCGCGGCACCAAGAAGCAACTTGCA
TCTAATGTGGCCGTTTTAGAGCTAGAAATAGCAAGTTAAAATAAGGCTAGTCCG
TTATCAACTTGAAAAAGTGGCACCGAGTCGGTGCCAACATTTTTTTTGTCCTTCTG
TTTTTTTAGTCAGTCTCTTTTTTCAGAAGTACAACATCTTTTTTTTGTCCTTCTGTT
TTTTTAGTCAGTCTTTTTTCAGAAGTACTCTATGTGATATCTTCGTTCTGGGAAAT
GTCTGTCTGTCTACAACCCATAATTATATTTGCAATCACACATCTAATATCTCTGT
GACAAGACAGCCGAACAACCTAGGTAAGATTAATTAACCCGGGGACCAAGCCCG
TTATTCTGACAGTTCTGGTGCTCAACACATTTATATTTATCAAGGAGCACATTGTT
ACTCACTGCTAGGAGGGAATCGAACTAGGAATATTGATCAGAGGAACTACGAGA
GAGCTGAAGATAACTGCCCTCTAGCTCTCACTGATCTGGGTCGCATAGTGAGATG
CAGCCCACGTGAGTTCAGCAACGGTCTAGCGCTGGGCTTTTAGGCCCGCATGATC
GGGCTTTTGTCGGGTGGTCGACGTGTTCACGATTGGGGAGAGCAACGCAGCAGT
TCCTCTTAGTTTAGTCCCACCTCGCCTGTCCAGCAGAGTTCTGACCGGTTTATAAA
CTCGCTTGCTGCATCAGACTTGGATGGCCAATGCGAGATGAGTTTTAGAGCTAG
AAATAGCAAGTTAAAATAAGGCTAGTCCGTTATCAACTTGAAAAAGTGGCACCG
AGTCGGTGCTTTTTTTCCGCGGCACGTCTCGAGCCCGGGTTAATTAAATTGGATG
ATGACTCTAGATAACGCAGGATCCACTAGTAACGGCCGCCAGTGTGCTGGAATT
GCCCTTGGATCATGAACCAACGGCCTGGCTGTATTTGGTGGTTGTGTAGGGAGAT
GGGGAGAAGAAAAGCCCGATTCTCTTCGCTGTGATGGGCTGGATGCATGCGGGG
GAGCGGGAGGCCCAAGTACGTGCACGGTGAGCGGCCCACAGGGCGAGTGTGAG
CGCGAGAGGCGGGAGGAACAGTTTAGTACCACATTGCCCAGCTAACTCGAACGC
GACCAACTTATAAACCCGCGCGCTGTCGCTTGTGTGATAGTAGTTAGTGCCGCG
TGTTTTAGAGCTAGAAATAGCAAGTTAAAATAAGGCTAGTCCGTTATCAACTTGA
AAAAGTGGCACCGAGTCGGTGCTTTTTTTGTCCCTTCGAAGGGCAATTCTGCAGA
TATCCATCACACTGGCGGCCGCTCGAGGTCGAGGGTATCGATAAGCTTGAATTCG
ACCCAGCTTTCTTGTACAAAGTTGGCATTATAAAAAATAATTGCTCATCAATTTG
TTGCAACGAACAGGTCACTATCAGTCAAAATAAAATCATTATTTG
SEQ ID NO: 26
CACCTGATTGTTTCTCACT
SEQ ID NO: 27
ACTTGCATCTAATGTGGCC
SEQ ID NO: 28
GATGGCCAATGCGAGATGA
SEQ ID NO: 29
ATAGTAGTTAGTGCCGCGT

Mfw3-A coding sequence (SEQ ID NO: 36), with the portion used for the Mfw-3/Mfw-5 hairpin described in Example 2 depicted in bold (SEQ ID NO: 54). Exemplary guide targeting sequences (SEQ ID NOs: 131-134) are shown in italics

ATGGGAGGAGGAGATTATCACCAGCAGAGCCTCATCGGCGGTGCGGCTGTTCAT
GGCCATGGAGGGGGCACCGTGGAGGCTGCGCTGAGGCCGCTCGTCGGCGGCT [symbol] [symbol]
GTACTGGCGGCTCTCTCCTGACCAGAGGTTC
TTGGAGATGGCGGGTTTTTGCTGCAGCGCCGAGTTCGAGGCGCAGGTGGCC
ACGCTCGCCGACGTCCCTTGCTCCATCCCTCTTGACTCCTCCTCCATCGGGA
TGCACGCTCAGGCGCTACTGTCGAACCAGCCAATCTGGCAGAGCAGCGGCG
GGGCGCCGGGTCCGGATCTCCTCACGGGCTACGAGGCTG [symbol] [symbol] [symbol] [symbol]
CGGGATCGTCGAGCTCTTC
GCGTCGAGATATATGGTCGAGGAGCAGCAGATGGCGGAGCTGGTCATGGCG
CAGTGCGGTGGCGGTGGGCAGGGGTGGCAGGAGACGGAGGCGCAGGGGTT
CGCGTGGGACGCGGCGGCGGCGGCAGACTCGGGGCGGCTCTACGCGGCGGCG
TCGCTCAACCTGTTCGACGGCGCCGGGGGAAGCGGCTCCGGCGAGCCGTTCCTG
GCGGGAGTGCAGGACGACGGCGCGGCGGGCGTGGGGTGGCAGTACGCGGCGGA
GAGCAGCGAGCCGCCGTCGACAGTGGCGCAGGAGCATCAGCAGCTGCACGGCTC
GGGCGTGGGGAGGGCAGATTCAGGGTCGGAGGGGAGCGATATGCAGCTGGGGG
ACCCCGACGACGACGGCAACGGCGAGACGCAGAGGGGCTCCGGCAAAGACGGC
AAAGACGCAGAGGGGAAGCGGCAGCAGTGCAAGAACCTCGAGGCGGAGCGGAA
GCGGCGCAGGAAGCTCAACGACCGCCTGTACAAACTCCGGTCCCTCGTCCCCAA
CATTACTAAGATGGACCGGGCGTCGATCCTCGGGGACGCGATCGACTACATCGTG
GGGCTGCAGAAGCAGGTGAAGGACCTGCAGGACGAGCTGGAGGACCCGAACCC
GCCGGGGGTCACCGGCGGCGACAGCAAGGCCCCCGACGTGCTCCTCGACGACCA
CCCGCCGCCGGGCCTCGACAACGACGAGGACTCGCCGCAGCAGCAGCCGTTCCC
GTCGGCCGGCGGGAAGCGGCCCCGGAAGGAGGAGGCCGGCGACGAGGAGGAGA
AGGAGGCGGAGGACCAGGACATGGAGCCGCAGGTGGAGGTCCGGCAGGTGGAG
GGGAAGGAGTTCTTCCTGCAGGTGCTCTGCTCCCACAAGTCCGGGCGCTTCGTCC
GCATCATGGACGAGATCGCCGCCCTCGGCCTCCAGATCACCAGCGTCAACGTCA
CCTCCTACAACAAGCTCGTCCTCAACGTCTTCCGGGCCGTCATGAAGGACAACGA
GGCGGCGGTGCCGGCGGACAGGGTGAGGGACTCGCTGCTGGAGGTGACGAGGG
AGATGTACGGCGGGGCCGGGGCGTGGTCGTCCCCGGTCCCTCCGCCGCCGCTGA
CAAACGCGAAGCTCGATGGTATGGACGGGCAGGCGGTGCCGACGGTGGCCGGG
GAGCACTACCAGCTGCACCACCAGGTGCTGGGAGGATATCATCACCAGCATCTG
CAGTACCTCGCCATGGATTGA
SEQ ID NO: 131
CCCACGGCTGGGACTACTGCAT
SEQ ID NO: 132
CCTCCAGCGGCGGCGAGAAGAC
SEQ ID NO: 133
GGCTCCTCGTCCCCGTCGCCGG
SEQ ID NO: 134
CCTCGGGGACGCGATCGACTAC

Mfw3-B coding sequence (SEQ ID NO: 37), with the portion used for the Mfw-3/Mfw-5 hairpin described in Example 2 depicted in bold (SEQ ID NO: 55). Exemplary guide targeting sequences (SEQ ID NOs: 135-138) are shown in italics

ATGGGAGGAGGAGATTATCACCAGCAGAGCCTCAACGGCGGTGCGGCTGTTCAT
GGGCATGGAGGGGGAGGGGGCGGCACCGTGGAGGCTGCGCTGAGGCCGCTCGT
CGGCGGCT [symbol] [symbol] CTACTGGCGGCTCTCTCCTGACC
AGAGGTTCTTGGAGATGGCGGGGTTTTGCTGCAGCGCCGAGTTCGAGGCGC
AGGTGGCCACGCTCGCCGACGTGCCTTGCTCCATCCCTCTTGACTCCTCCTC
CGTCGGGATGCACGCTCAGGCGCTACTGTCGAACCAGCCAATCTGGCAGAG
CAGTGGCGGGTCGCCGGGCCCGGATCTCCTCACGGGCTACGAGGCTG [symbol] [symbol] [symbol] [symbol] [symbol]
CGGGATCGTCG
AGCTCTTCGCGTCGAGATATATGGCGGAGGAGCAGCAGATGGCTGAGCTGG
TCATGGCGCAGTGCGGTGGCGGTGGGCAGGGGTGGCAGGAGACGGAGGCG
CAGGGGTTCGCGTGGGACGCGGCGGCGGCAGACCCCGGGCGGCTCTACGCGG
CGGCGTCGCTCAACCTATTCGACGGCGCCGGGGGAAGCGGCTCCGGCGAGCCGT
TCCTGGCGGGAGTGCAGGAGGATGGCGCGGCGGGCGTGGGGTGGCAGTACGCG
GCAGAGAGCAGCGAGCCGCCGTCGACGGTGGCGCAGGAGCATCAGCAGCTGCA
CGGCTCGGGCGTGGGGAGGGCAGATTCGGGGTCGGAGGGGAGCGATATGCAGCT
GGGAGACCCCGACGACGAAGTCGACGGCGAGACGCAGAGGGGCTCCGGCAAAG
ACGGCTGCGGGAAGCGGCAGCAGTGCAAGAACCTCGAGGCGGAGCGGAAGCGG
CGGAAGAAGCTCAACGAACGCCTCTACAAGCTCCGGTCCCTCGTCCCAAACATT
ACCAAGATGGACCGGGCGTCGATCCTCGGGGACGCGATCGACTACATAGTGGGGC
TGCAGAAGCAGGTGAAGGACCTGCAGGACGAGCTGGAGGACCCAAACCTGCCG
GGGATCACCGGCGGCGACAGCAAGGCCCCCGACGTGCTCCTCGACGACCACCCG
CCGCCGGGCCTCGACAACGACGAGGACTCGCCGCAGCAGCAGCCGTTCCCGTCC
GCCGGCGGCAAGCGGCTCCGGAAGGAGGAGGCGGGCGACGAGGAGGAGAAGGA
GGCGGAGGACCAGGACATGGAGCCGCAGGTGGAGGTCCGGCAGGTGGAGGGGA
AGGAGTTCTTCCTACAGGTGCTGTGCTCCCACAAGTCCGGGCGCTTCGTCCGCAT
CATGGACGAGATCGCCGCCCTCGGCCTCCAGATTACCAGCATCAACGTCACCTCC
TACAACAAGCTCGTCCTCAACGTCTTCCGCGCCGTCATGAAGGACAACGAGGCG
GCGGTGCCGGCGGACAGGGTGAGGGACTCGCTGCTGGAGGTGACCAGGGAGAT
GTACAGCGGGGGCGGCACGTGGTCGTCCCCGGTCCCTCCGCCGCCGCCGACAAA
CGCAAAGCTCGATGGCATGGACGGGCAGGCGGTGCCGGCGGCCGCCGGGGACC
ACTACCAGCTGCACCACCAGGTGCTGGGAGGATATCATCACCAGCATCTGCAGT
ACCTCGCCATGGATTGA
SEQ ID NO: 135
CCCACGGCTGGGACTACTGCAT
SEQ ID NO: 136
CCTCCAGCGGCGGCGAGAAGAC
SEQ ID NO: 137
GGCTCCTCGTCCCCGTCGCCGG
SEQ ID NO: 138
CCTCGGGGACGCGATCGACTAC

Mfw3-D coding sequence (SEQ ID NO: 38), with the portion used for the Mfw-3/Mfw-5 hairpin described in Example 2 depicted in bold (SEQ ID NO: 56). Exemplary guide targeting sequences (SEQ ID NOs: 139-142) are shown in italics.

ATGGCAGGAGGAGACTATCACCAGCAGAGCATCATCGGCGGCCGTGCGGCTGTT
CATGGCCATGGAGGGGGAGGCGGCGGCACCGTGGAGGCTGCGCTCAGGCCGCT
CGTCGGCGGCG [symbol] [symbol] CTACTGGCGGCTCTCTCCTG
ACCAGCGGTTCTTGGAGATGACGGGGTTCTGCTGCAGCGCGGAGTTCGAGG
CGCAGGTGGCCACGCTCGCCGACGTCCCTTCCTCCATCCCTCTCGACTCCTC
CTCCATCGGGATGCACGCTCAGGCCCTGCTGTCGAACCAGCCGATCTGGCA
GAGCAGCGGCGGGGCGCCGGGTCCGGATCTACTCACGGGCTACGAGGCTT [symbol] [symbol] [symbol] [symbol] [symbol]
CGGCATCGT
CGAGCTCTTCGCTTCAAGATACATGGCGGAGGAGCAGCAGATGGCGGAGCT
GGTCATGGCGCAGTGCGGCGGCGGTGGGCAGGGATGGCAGGAGACGGAGG
CGCAGGGGTTTGCGTGGGACGCGGCAGCGGCAGACCCGGGGCGGCTCTACGC
GGCGGCGTCGCTCAACCTGTTCGACGGCGCCGGGGGAAGCGGCTCGGGCGAGCC
GTTCCTGGCGGGAGTGCAGGAGGACGGCGCGGCGGGCGTGGGTTGGCAGTACGC
GGCAGAGAGCAGCGAGCCGCCGTCGACGGTGGCGCAGGAGCATCAGCAGCTGC
ACGGCTCGGGCGTGGGGAGGGCGGACTCGGGGTCGGAGAGGAGTGACATGCAG
CTGGGGGACCCCGACGACAACGTCGACGGCGAGACGCAGAGGGGCTCCGGCAA
AGACGGCGGCGGGAAGCGGCAGCAGTGCAAGAACCTCATCGCGGAGCGGAAGC
GGCGCAAGAAGCTCAACAACCGCCTCTACACGCTCCGGTCCCTCGTCCCCAACAT
CACCAAGATGGACCGTGCGTCGATCCTCGGGGACGCGATCGACTACATCGTGGGG
CTGCAGAAGCAGGTGAAGGACCTGCAGGACGAGCTGGAGGACCCGAACCCGCC
GGGGGTCACCGGCGGCCACAGCAAGGCCCCCGACGTGCTCCTCGACGACCACCC
GCCGCCGGGCCTCGACAACGACGAGGACTCGCCGCAGCAGCAGCCGTTCCCGTC
CGCCGCCGGCAAGCGGCCCCGGAAGGTGGAGGCGGGCGAGGAGGAGGAGAAGG
AGGCGGAGGACCAGGACATGGAGCCGCAGGTGGAGGTCCGGCAGGTGGAGGGG
AAGGAGTTCTTCCTGCAGGTGCTGTGCTCCCACAAGTCCGGGCGCTTCGTCCGCG
TCATGGACGAGATCGCCGCCCTCGGCCTCCAGATCACCAGCGTCAACGTCACCTC
CTACAACAAGCTCGTCCTCAACGTCTTCCGCGCCGTCATGAAGGACAACGAGGC
GGCGGTGCCGGCGGACAGGGTGAGGGACTCGCTGCTGGAGGTGACGAGGGAGA
TGTACGGCGGGGGCGGCGCGTGGTCGTCCCCGCTCCCCCCGCCGCCGCCGACGA
ACGCGAAGCTCGATGGCATGGACGGGCAGGCGGTGCCGGCGGCGGCCGGGGAC
CACTACCAGCTGCACCACCAGGTGCTGGGAGGATATCACCACCAGCATCTGCAG
TACCTCGCCATGGATTGA
SEQ ID NO: 139
CCCACGGCTGGGACTACTGCAT
SEQ ID NO: 140
CCTCCAGCGGCGGCGAGAAGAC
SEQ ID NO: 141
GGCTCCTCGTCCCCGTCGCCGG
SEQ ID NO: 142
CCTCGGGGACGCGATCGACTAC

Mfw5-A coding sequence (SEQ ID NO: 129), with the portion used for the Mfw-3/Mfw-5 hairpin described in Example 2 depicted in bold (SEQ ID NO: 57). Exemplary guide targeting sequences (SEQ ID NOs: 143-146) are shown in italics.

ATGACAGGATCTTTGACCCATGATTCTTCTCTGGCTCCTAAATGCAACGACAACACAAAT
ATTGAGCTACAGAGATTCAAGGTGCAGTCGTTTTCTGCAGATATCCTTTCTGATTCGACCAA
TCTTTCTTCTGAAGCTGCAAGAGCAATCAACCACCTTCAGCATCAACTAGGAATTGGTTT
GGAGCAGGATATGCGACCAGTGGAAACTGCGA [symbol] [symbol] CATTC
AAGACCAAATAATCAACCATCAGCTTAGCGAAGATCCACAAAACATATTGGTGCAA
CAACAGATTCAACAGTATGATGCTGCGCTTTATCCAAACAGTGGTTACACA [symbol] [symbol]
TTCTCCACTGCACTGTGGCTCCAGTGTTCCCTCCAACAGCAT
CAGTTTTTGGTGATACAGCACTAAGTGGTGGTACCAACTATTTGGATCTTAATGATG
AGTTTACAGGAGTGGCAGCAATTCCTGACAGTGGATTAATGTACACTAGTGATCCG
GCATTGCAGTTAGGGTACCATGCTGCCCAGTCTCACGCACTAAAGGATATCTGCCA
TTCACTGCCGCAAAATTATGGGCTGTT [symbol] [symbol] GCCATCCTT
GGGGTTGGAAGTGTCGGAGGAGATCTTTTTCAGGATATGGATGACAGGCAATTTGATA
CTGTACTGGAGGGCAGAAGAGGGAAGGGTGACTTCGGAAAGGGAAAAGGAAAAGCTAA
CTTTGCGACAGAGAGAGAGAGGAGGGAACAGCTAAATGTGAAGTATAAGACTTTAAGA
ATGCTCTTCCCCAATCCTACCAAGAATGACAGGGCTTCAGTAGTAGGTGATGCCATTGAA
TACATAGATGAGCTGAATCGAACAGTGAAGGAACTGAAGATCCTAGTGGAACAGAAGTG
GCATGGGACTAATAGGAGAAAGATAAGAAAGTTGGATGAAGAGGCCGCTGCTGATGGT
GAAAGCTCATCGATGAGGCCAATAAGGGATGAGCAAGACAATCAGCTTGATGGGGCCAT
AAGAAGCTCATGGGTTCAGAGGAGGTCCAGGGAGTGCCATGTTGATGTTCGCATAGTGG
AAAATGAAATAAACATCAAGCTCACAGAAAAGAAGACGACCAACTCCTCCCTGCTTCAT
GTTGCAAAGGTTCTTGATGAATTCCATCTTGAGATCATCCATGTGGTTGGAGGGATTATT
GGTGATCACTACATATTCATGTTTAACACTAAGGTGTCTGAAGGTTCCTCAATTTATGCTT
GTGCAGTGGCAAAGAGGATCCTTCAAGCAGTGGATGCACAACACCAGGCACTTGACATA
TTCAACTAG
SEQ ID NO: 143
ATTGAGCTACAGAGATTCAAGG
SEQ ID NO: 144
CCTGGGATACTTCTATCTGCAC
SEQ ID NO: 145
CCAGCACCTGATCTCTTAAACC
SEQ ID NO: 146
CCCCAGTGAGGATGAAAGAGAT

Mfw5-B coding sequence (SEQ ID NO: 130), with the portion used for the Mfw-3/Mfw-5 hairpin described in Example 2 depicted in bold (SEQ ID NO: 58). Exemplary guide targeting sequences (SEQ ID NOs: 147-150) are shown in italics.

ATGGGACTTCTCTACACGGAAGAACAGACAGCCACATTGCATAGCTTAAAACTC
CACGGCTCTACCTCTTTTGCAACAACCAAAACAGCCAGGCCAACTGCAATTNNN
NNNCATGATTCTTCTCTGGCTCCTAAATGCAACGACAACACAAATATTGAGCTACA
GAGATTCAAGGTGCAGTCGTTTTCTGCAGATATCCTTTCTGATTCGACCAATCTTTC
TTCTGAAGCTGCAAGAGCGATCAACCACCTCCAGCATCAACTAGGAATTGGTTTG
GAGCAGGATATGCCGCCAGTGGGAACTGCGA [symbol] [symbol]
ATTCAAGACCAAATTATCAACCATCAGCTTAGCGAAGATCCACAAAACATAT
TGGTGCAACAACAGATTCAACAGTATGATGCTGCGCTTTATCCAAACAGTGG
TTACACA [symbol] [symbol] TTCTCCACTGCACTGTGGCTCCAGT
GTTCCCTGCAACAGCATCAGTCTTTGGTGATACAGCACTAAGTGGTGATACC
AACTATTTGGATCTTAATGGTGAGTTTACAGGAGTGGCAGCAATTCCTGACA
GTGGATTAATGTACACTAGTGATCCAGCATTGCAGTTAGGGTACCATGCTGC
CCAGTCTCACGCACTAAAGGATATCTGCCATTCACTGCCGCAAAATTATGGG
CTCTT [symbol] [symbol] GTCATGCTTGGGGTTGGAAGTGTCGG
AGGAGATCTTTTTCAGGATATAGATGACAGGCAATTTGATACTGTACTGGAGGGC
AGAAGAGGAAAGGGTGAGTTCGGAAAAGGAAAAGGAAAAGCTAACTTTGCGAC
TGAGAGAGAGAGGAGGGAACAACTCAATGTGAAGTATAAGACGTTAAGAATGCT
CTTCCCCAACCCTACCAAGAATGACAGGGCTTCAGTAGTAGGTGATGCCATTGAA
TACATAGATGAGCTGAATCGAACAGTGAAGGAACTGAAGATCCTAGTGGAACAG
AAGTGGCATGGGACTAATAGGAGAAAGATAAGAAAGTTGGATGAAGAGGCGGC
TGCTGATGGTGAAAGCTCATCGATGAGGCCAATGAGGGATGAGCAAGACAATCA
GCTTGATGGGGCCATAAGAAGCTCATGGGTTCAGAGGAGGTCCAGGGAGTGCCA
TGTTGATGTTCGCATAGTGGAAAATGAAATAAACATCAAGCTCACAGAAAAGAA
GAAGACCAACTCCTCCCTGCTTCATGTTGCAAAGGTTCTTGATGAATTCCATCTT
GAGATCATCCATGTAGTTGGAGGGATTATTGGTGATCACTACATATTCATGTTTA
ACACTAAGGTGACTGAAGGTTCCTCAGTTTATGCTTGTGCAGTGGCAAAGAGGAT
CCTTCAAGCAGTGGATGCACAACACCAGGCACTTGACATATTCAACTAG
SEQ ID NO: 147
ATTGAGCTACAGAGATTCAAGG
SEQ ID NO: 148
CCTGGGATACTTCTATCTGCAC
SEQ ID NO: 149
CCAGCACCTGATCTCTTAAACC
SEQ ID NO: 150
CCCCAGTGAGGATGAAAGAGAT

Mfw5-D coding sequence (SEQ ID NO: 41), with the portion used for the Mfw-3/Mfw-5 hairpin described in Example 2 depicted in bold (SEQ ID NO: 59). Exemplary guide targeting sequences (SEQ ID NOs: 151-154) are shown in italics.

ATGCCACCAGTGGAAACTGCGA [symbol] [symbol] CATTCAAGAC
CAAATAATCAACCATCAGCTTAGCGAAGATCCACAAAACATATTGGTGCAAC
AACAGATTCAACAGTATGATGCTGCGCTTTATCCAAACAGTGGTTACACA [symbol] [symbol] [symbol] [symbol]
TTCTCCACTGCACTGTGGCTCCAGTGTTCCCTGC
AACAGCATCAGTCTTTGGTGATACAGCACTAAGTGGTGGTACCAACTATTTG
GATCTTAATGGTGAGTTTACAGGAGTGGCAGCAATTCCTGACAGCGGATTA
ATGTACACTAGTGATCCGGCATTGCAGTTAGGGTACCATGCTGCCCCGTCTC
ACGCACTAAAGGATATCTGCCATTCACTGCCGCAAAATTATGGACTGTT [symbol] [symbol] [symbol]
GTCATGCTTGGGGTTGGAAGTGTCGGAGGAGATC
TTTTTCAGGATATGGATGACAGGCAATTTGAAACTGTACTGGAGGGCAGAAGAG
GGAAGGGTGAGTTCGGAAAGGGAAAAGGAAAAGCTAACTTTGCGACTGAGAGA
GAGAGGAGGGAACAGCTAAATGTGAAGTATAAGACTTTAAGAATGCTCTTCCCC
AATCCTACCAAGAATGACAGGGCTTCAGTAGTAGGTGATGCCATTGAATACATA
GATGAGCTGAATCGAACAGTGAAGGAACTGAAGATCCTAGTGGAACAGAAGTG
GCATGGGACTAATAGGAGAAGGACAAGAAAGTTGGATGAAGAGGCGGCTGCTG
ATGGTGAAAGCTCATCGATGAGGCCAATGAGGGATGAGCAAGACAATCAGCTTG
ATGGGGCCATAAGAAGCTCATGGGTTCAGAGGAGGTCCAGGGAGTGCCATGTTG
ATGTTCGCATAGTGGAAAATGAAATAAACATCAAGCTCACAGAAAAGAAGAAG
GCCAACTCCTCCCTGCTTCATGTTGCAAAGGTTCTTGACGAATTCCATCTTGAGAT
CATCCATGTGGTTGGAGGGATTATTGGTGATCACTACATATTCATGTTTAACACT
AAGGTGACTGAAGGTTCCTCAGTTTATGCTTGTGCAGTGGCAAAGAGGATCCTTC
AGGCAGTGGATGCACAACACCAGGCACTTGACATATTCAACTAG
SEQ ID NO: 151
CCTGGGATACTTCTATCTGCAC
SEQ ID NO: 152
CCAGCACCTGATCTCTTAAACC
SEQ ID NO: 153
CCAGCACCTGATCTCTTAAACC
SEQ ID NO: 154
CCCCAGTGAGGATGAAAGAGAT

Cas9 and sgRNA sequences can be expressed either stably or transiently in a cell in order to generate the deactivating modifications described herein. In one aspect of any of the embodiments, described herein is a wheat cell comprising 1) an exogenous Cas9 protein and/or an exogenous nucleic acid encoding a Cas9 protein: and 2) at least one sgRNA capable of specifically hybridizing with at least one Mfw and/or Mpew gene sequence under cellular conditions or a nucleic acid encoding such an sgRNA. In some embodiments of any of the aspects, the sgRNA can comprise a sequence selected from SEQ ID NOs: 22-29 and/or 131-154. In some embodiments of any of the aspects, the 1) exogenous nucleic acid encoding a Cas9 protein: and 2) the nucleic acid encoding at least one sgRNA capable of specifically hybridizing with at least one Mfw and/or Mpew gene sequence under cellular conditions are provided in a vector or vector(s). In some embodiments of any of the aspects, the vectors are transient expression vectors. In some embodiments of any of the aspects, the 1) exogenous nucleic acid encoding a Cas9 protein: and 2) the nucleic acid encoding at least one sgRNA capable of specifically hybridizing with at least one Mfw and/or Mpew gene sequence under cellular conditions are integrated into the genome. It is contemplated herein that similar approaches to vector delivery, transient expression, and/or stable integration can also be utilized in embodiments relating to, e.g., inhibitory RNAs, TALENs, and/or ZFNs.

In one aspect of any of the embodiments, described herein is a nucleic acid encoding at least one sgRNA capable of specifically hybridizing with at least one Mfw and/or Mpew gene sequence, e.g., under cellular conditions. In one aspect of any of the embodiments, described herein is a nucleic acid encoding at least one sgRNA capable of targeting Cas9 or a related endonuclease to at least one Mfw and/or Mpew gene sequence, e.g., under cellular conditions. In some embodiments of any of the aspects, the sgRNA can comprise a sequence that can specifically hybridize, in the cell, to a sequence selected from SEQ ID NOs: 1-12. In some embodiments of any of the aspects, the sgRNA can comprise a sequence selected from SEQ ID NOs: 22-29 and/or 131-154. In some embodiments of any of the aspects, the nucleic acid further encodes a Cas9 protein. In some embodiments of any of the aspects, the nucleic acid is provided in a vector. In some embodiments of any of the aspects, the vector is a transient expression vector.

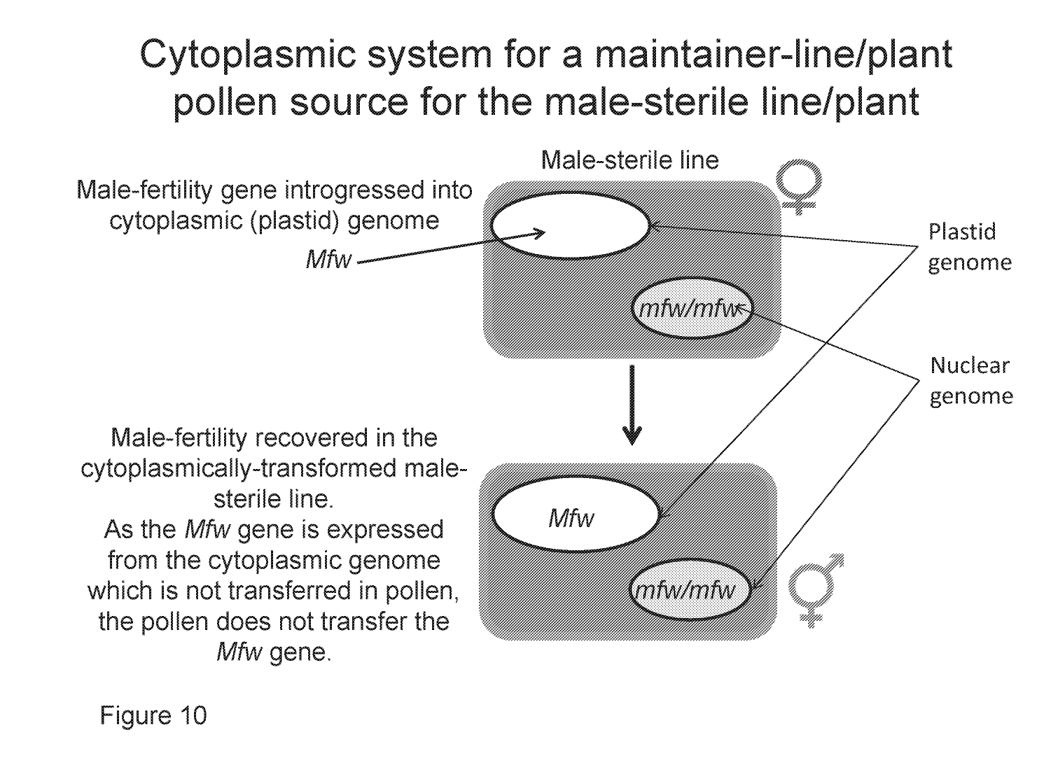

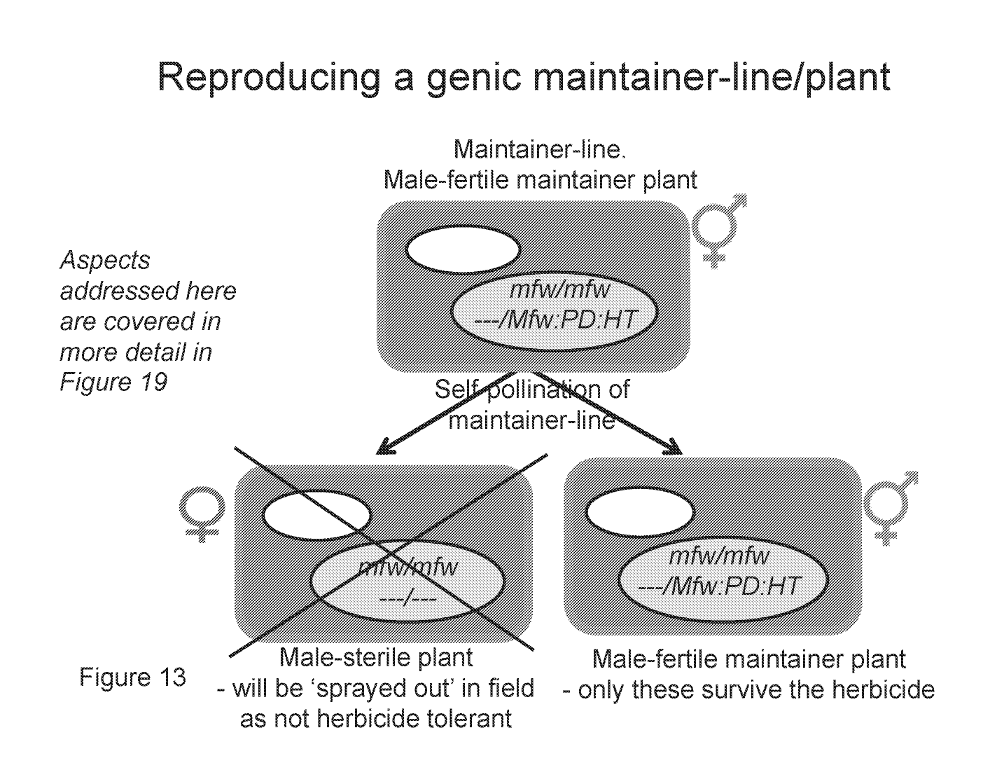

Further described herein are methods and compositions relating to a ‘maintainer line’ for the male-sterile(s) plants described herein. In one aspect, the deactivated genes can be introgressed into the cytoplasmic genome of the male-sterile lines. This will produce a male-fertile phenotype which is not pollen-transmitted to the male-sterile line it fertilises, enabling maintenance of the male-sterile lines. An illustrative example of this approach is depicted schematically in FIG. 10. This maintainer line then allows the maintenance of the male-sterility by crossing with the male sterile line. The pollen is viable on the maintainer line allowing seed set of/on the male-sterile line, but, after sowing such seed, the resulting plant is still male-sterile, because the wild-type Mfw is plastid-located in the maintainer line and therefore Mfw is not inherited through its pollen (FIG. 14).

Accordingly, in one aspect, described herein is a wheat plant and/or seed comprising a) a deactivating modification of each nuclear copy of one or more Mfw and/or Mpew genes and b) a nucleic acid encoding an exogenous wild-type sequence of at least one of the Mfw and/or Mpew genes, wherein the nucleic acid is located in the cytoplasmic genome. In some embodiments, each member of a gene family can be deactivated and the maintainer line can comprise a nucleic acid encoding an exogenous wild-type sequence of one member of the gene family, e.g., the male-sterile phenotype can be rescued by restoring expression of one member of a functionally redundant group.

Alternatively, a maintainer line can be generated by introducing a maintainer line construct into the male sterile cell or plant. In some embodiments, such construct can comprise 1) an Mfw gene (appropriate to counteract the mfw male-sterility gene concerned) 2) a “pollen death” PD gene and 3) a herbicide tolerant (hereinafter ‘HT’)—or other appropriate selectable marker gene—to enable deselection of non-transformants (together this is referred to herein as a Mfw/PD/HT construct).

As used herein, a Mfw/PD/HT construct is a gene or group of genes that, when introduced, in a hemizygous manner, into a plant with a male-sterile phenotype due to deactivation of a Mfw and/or Mpew gene as described herein, conveys a meiosis-competent phenotype that results in post-meiosis pollen death or non-viability in the gamete receiving the hemizygous Mfw/PD/HT construct. Non-viability here, is the lack of ability, for whatever reason, to effect fertilisation of a wheat ovule. The transgene-hemizygote pollen mother cell will, after meiosis, produce pollen sperm cells which, 50:50, contain either the transgene or do not. The pollen sperm cells with the transgene will die or be non-viable; those without it will survive and be viable for fertilisation. The surviving pollen sperm cells can then self-pollinate their parent plant or, after dispersal, cross-pollinate another plant, eg a male-sterile F1 parent line plant. In the latter case, because the transgene construct with its dominant male-fertility, Mfw gene has been eliminated by its post-meiosis Mfw/PD/HT gene, the remaining pollen will only contain the recessive mfw male-sterility gene and will not transfer the Mfw male-fertility of the fully fertile parent.

In embodiment of any of the aspects, a Mfw/PD/HT construct comprises a) nucleic acid comprising a wild-type sequence of at least one of the Mfw and/or Mpew genes which have been deactivated, wherein the deactivating modifications of the Mfw and/or Mpew are found in the coding sequences themselves (e.g., not by introducing an inhibitory nucleic acid) and b) an inhibitory nucleic acid targeting a post-meiosis-expressed pollen viability gene such as Mfw1, wherein the inhibitory nucleic acid is under the control of a pollen-specific promoter, e.g., a late-pollen specific promoter. The pollen specific promoter can avoid the gene being activated earlier, eg in the tapetum, when all pollen cells might be affected rather than just those with the transgene.)

In some embodiments of any of the aspects, a Mfw/PD/HT construct can comprise a) a pollen-cytotoxic gene under the control of a pollen-specific promoter and b) a nucleic acid comprising a wild-type sequence of at least one of the Mfw and/or Mpew genes which have been deactivated, wherein the deactivating modifications of the Mfw and/or Mpew are found in the coding sequences themselves (e.g., not by introducing an inhibitory nucleic acid) and, c) an HT gene. The hemizygous female megasporocyte will produce, 50:50, ovules which contain the construct or do not. Once fertilised by 100% mfw pollen the resultant embryos and seed will be, 50:50, transgenic or not; the former will be male-fertile due to expression of the construct's Mfw gene, the latter will be male-sterile due to the lack of Mfw gene expression. In a seed production field intended to produce pollinators for the male-sterile line, the 50% male-sterile plants are a hindrance and if an HT gene is present, the male-sterile plants can be eliminated by spraying the seed production field with the herbicide for which the transgene is tolerant. The embodiments described herein which relate to use of an HT gene can provide certain advantages over other approaches, e.g., the use of a seed endosperm pigmentation gene. Because of the relative opaqueness of wheat's seed coat and small size of wheat seeds, colour separation approaches can incur high costs without achieving optimal accuracy. Use of HT genes in wheat plants as described herein is contemplated to provide increased accuracy and lower cost per acre as compared to the use of seed coat pigmentation approaches. Nevertheless, in some embodiments, for extra confidence of lack of transgenes in the male-sterile for example, a color selectable marker gene can be added to the construct.

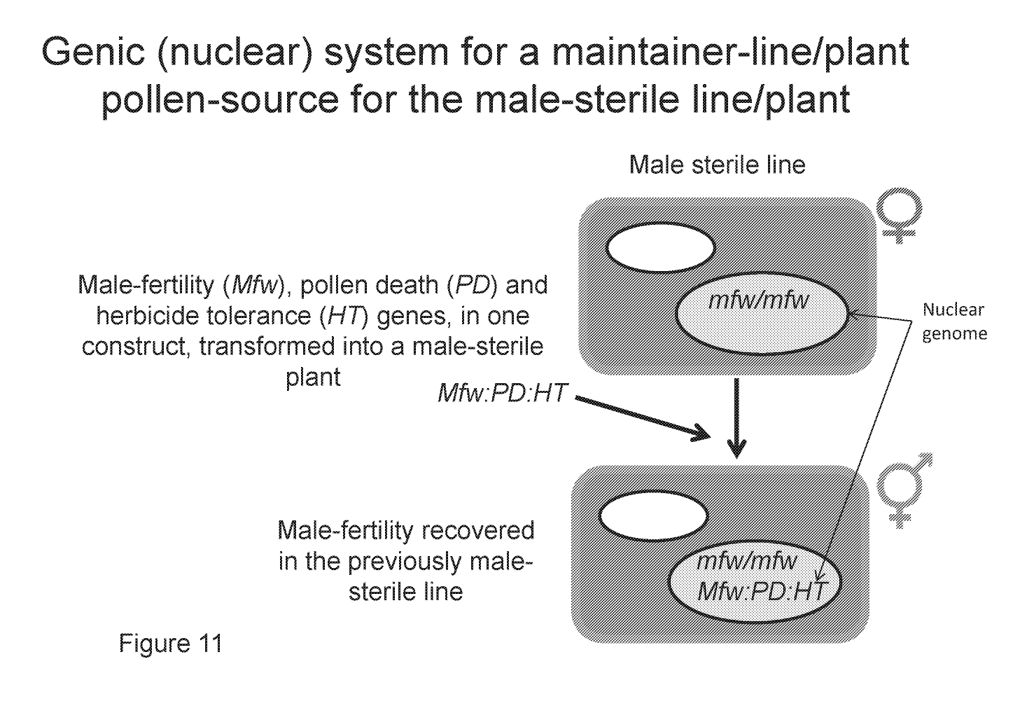

An illustrative example of this approach is depicted schematically in FIG. 11. Exemplary pollen-specific promoters for use in wheat are known in the art and can include, by way of non-limiting example, pPG47 and TaPSG719 (see, e.g, Chen, L., Tu, Z., Hussain, J. et al. Mol Biol Rep (2010) 37: 737; which is incorporated by reference herein in its entirety). Exemplary pollen-cytotoxic genes are known in the art and can include alpha-amylase, barnase (see, e.g., Zhang et al Plant Physiology (2012) 159:1319-1334; which is incorporated by reference herein in its entirety, and orf288 (see, e.g, Jing et al. J. Exp. Bot. (2012) 63:1285-1295; which is incorporated by reference herein in its entirety). In some embodiments of any of the aspects, the pollen-cytotoxic gene is not an alpha-amylase gene, not an amylase gene, and/or has less than 60% sequence identity with the ms45 gene from Zea mays.

In some embodiments of any of the aspects, the nucleic acid comprising a wild-type sequence of at least one of the Mfw and/or Mpew genes can be operably linked to a promoter. In some embodiments of any of the aspects, the promoter operably linked to the nucleic acid comprising a wild-type sequence of at least one of the Mfw and/or Mpew genes can be an anther-specific promoter.

In some embodiments of any of the aspects, the HT gene can be a glyphosate-tolerance gene. In some embodiments of any of the aspects, the HT gene can be operably linked to a constitutive promoter.

In some embodiments of any of the aspects, a Mfw/PD/HT construct can be introduced into the genome, e.g., stably integrated at a location other than at the original Mfw and/or Mpew locus which was deactivated.

Accordingly, in one aspect of any of the embodiments, described herein is a wheat plant and/or seed comprising a deactivating modification of each nuclear copy of one or more Mfw and/or Mpew genes and further comprising a Mfw/PD/HT construct. In some embodiments, the Mfw/PD/HT construct is located in the nuclear genome.

In some embodiments of any of the aspects, the Mfw/PD/HT construct can further comprise an extra selection gene and/or selection construct, e.g., one that allows a seed comprising the Mfw/PD/HT construct to be distinguished from seeds not comprising the Mfw/PD/HT construct. In some embodiments of any of the aspects, the selection gene permits one to distinguish the seeds by visual and/or optical means, e.g., the selection gene can convey a non-standard color to the seed including to seed produced as a result of fertilisation by pollen containing the color-selection gene. In some embodiments of any of the aspects described herein, a plant, seed, and/or maintainer line as described herein can further comprise a selectable marker gene and/or selectable marker construct. The selectable marker gene and/or selectable marker construct can comprise a selectable marker, e.g. a marker that conveys an optically-detectable difference in seed coat color, under the control of a promoter which permits expression of the selectable marker gene at least in the endosperm. Thus, a seed or plant resulting from pollination with a pollen grain comprising selectable marker gene and/or selectable marker construct will express the selectable marker. Such markers can be selected against and/or screened against in order to provide a group of seeds and/or plants which do not comprise the selectable marker gene and/or construct, and thus also do not comprise the Mfw/PD/HT. Such an approach can prevent undesired dissemination of transgenic material. Exemplary selectable markers can include a blue aleurone (Ba) layer selectable marker gene. The Ba selectable marker gene and its use are known in the art, e.g., see U.S. Pat. No. 6,407,311. In some embodiments, the selectable marker construct can comprise multiple copies of the selectable marker, e.g., 2 copies, 3 copies, or more copies, and/or the selectable marker can be expressed by a strong promoter, e.g., to ensure desired levels of phenotypic penetrance and expression.

Maintainer lines comprising a Mfw/PD/HT construct permit the maintenance of the male-sterility by crossing with the male-sterile line. The maintainer line's pollen, containing only mfw alleles due to Mfw-containing pollen having been eliminated by the post-meiosis PD gene, is viable on the male-sterile line and enables seed set of the male-sterile line without transferring any Mfw male-fertility alleles (FIG. 12).

In some embodiments, each member of a gene family can be deactivated and the maintainer line can comprise an exogenous copy of one member of the gene family, e.g., the male-sterile phenotype can be rescued by restoring expression of one member of a functionally redundant group.

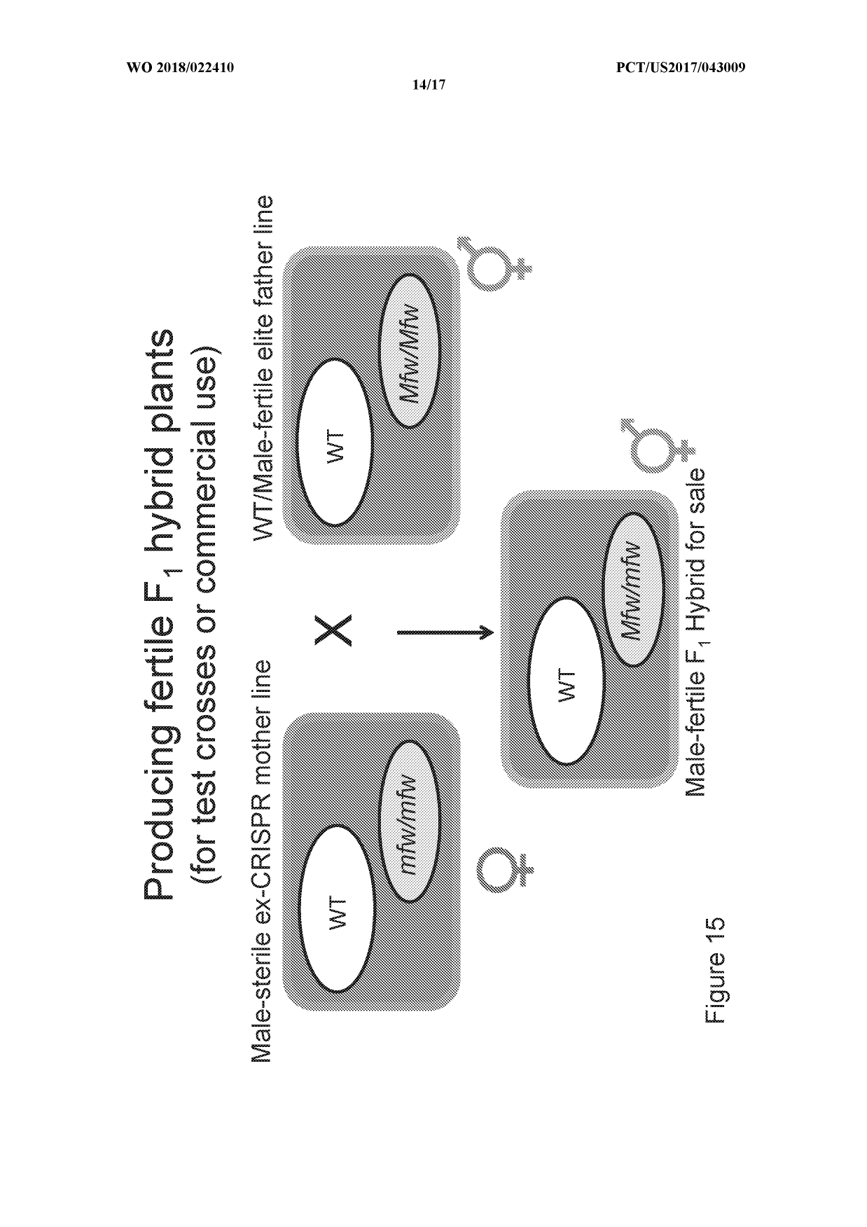

It is further contemplated herein that once male-sterile and maintainer material has been produced, the deactivated genes/alleles/characters and/or deactivating modifications can be transferred to elite standard lines by normal backcrossing (with appropriate marker-assisted selection for the male-sterile material) (FIG. 16).

The methods and compositions described herein provide a number of advantages over existing wheat technologies. For example, a low cost of final production; no special spraying of the intended male-sterile lines in potentially large-scale F1 seed production field to create the necessary male-sterile trait in the seed-producing parent; a low cost of breeding (many test-crosses can be made with wild-type, standard lines being potential pollinator lines (with wild-type dominant fertility), and no separate breeding programme to produce ‘final’ pollinator lines); the final F1 production and seed sold may not be classified as “genetically modified” under some jurisdictions' consumer guidelines or seed or GM regulations. For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level.

The terms “increased”, “increase”, “enhance”, or “activate” are all used herein to mean an increase by a statistically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

The degree of homology (percent identity) between a native and a mutant sequence can be determined, for example, by comparing the two sequences using freely available computer programs commonly employed for this purpose on the world wide web (e.g. BLASTp or BLASTn with default settings).

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments, a nucleic acid encoding an RNA or polypeptide as described herein can be introduced into a cell by, e.g., biolistic delivery.

In some embodiments, a nucleic acid encoding an RNA or polypeptide as described herein is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc. Exemplary vectors are known in the art and can include, by way of non-limiting example, pBR322 and related plasmids, pACYC and related plasmids, transcription vectors, expression vectors, phagemids, yeast expression vectors, plant expression vectors, pDONR201 (Invitrogen), pBI121, pBIN20, pEarleyGate100 (ABRC), pEarleyGate102 (ABRC), pCAMBIA, pUC-derived vectors, pSK-derived vectors, pGEM-derived vectors, pSP-derived vectors, pBS-derived vectors, the binary Ti plasmid (see, e.g., U.S. Pat. No. 4,940,838; which is incorporated by reference herein in its entirety), T-DNA, transposons, and artificial chromosomes.

As used herein, the term “expression vector” refers to a vector that directs expression of an RNA or polypeptide from sequences operably linked to transcriptional regulatory sequences on the vector. The term “operably linked” as used herein refers to a functional linkage between a regulatory element and a second sequence, wherein the regulatory element influences the expression and/or processing of the second sequence. Generally, “operably linked” means that the nucleic acid sequences being linked are contiguous and, where necessary to join two protein coding regions, contiguous and in the same reading frame. The regulatory sequence, e.g., a promoter, can be a constitutive, tissue-specific, and/or inducible promoter. The sequences expressed will often, but not necessarily, be heterologous to the cell. An expression vector may comprise additional elements, for example, the expression vector may have two replication systems, thus allowing it to be maintained in two organisms, for example in plant cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), and Current Protocols in Protein Science (CPPS), John E. Coligan (ed.), John Wiley and Sons, Inc., 2005; the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method of producing male-sterile wheat which comprises during the development of the flower:
   analysing the RNA-transcriptome of wheat stamen cells;
   analysing the RNA-transcriptome of wheat pistil cells;
   then comparing the two RNA-transcriptomes to identify one or more genes that at the time of flowering are preferentially expressed in stamens rather than pistils;
   selecting one or more Mfw genes so identified;
   inhibiting expression of at least one selected Mfw gene, so as to produce male-sterile wheat.
2. A method as paragraphed in paragraph 1 in which RNA-transcriptome analysis is carried out during meiosis.
3. A method as paragraphed in paragraphs 1 or 2 in which RNA-transcriptome analysis is carried out between stages 41 to 49 of the Zadoks scale, inclusive.
4. A method as paragraphed in any of paragraphs 1-3, wherein RNA-transcriptome analysis is carried out in juvenile flowers comprising both immature stamens and pistils.
5. A method as paragraphed in any of paragraphs 1 to 4 in which a selected Mfw gene codes for an amino-acid sequence identical, or having corresponding function and least 60%, preferably at least 90% or 95% identity, with any of SEQ ID NOs 1-6 and/or SEQ ID NOs: 30-35 or a sequence of a gene of Tables 1 or 2.
6. A method as paragraphed in any of paragraphs 1 to 5 in which the selected Mfw gene has the sequence shown in any of SEQ ID NOs 7-12, 36-41, and/or 129-130 or has at least 60%, preferably at least 90% or 95% identity therewith.
7. The method as paragraphed in any of paragraphs 1-6, wherein the selected Mfw genes are at least two of Mfw1, Mfw2, Mfw3, and Mfw5.
8. A method as paragraphed in any of paragraphs 1-67 in which the selected Mfw gene is deactivated by site-directed mutagenesis employing a site-specific nuclease.
9. A method as paragraphed in paragraph 8 in which the site-specific nuclease is CRISPR-Cas.
10. A method as paragraphed in either of paragraphs 8 or 9 in which the Mfw gene is deactivated by excision of at least part of a coding or regulatory sequence.
11. A method as paragraphed in any of paragraphs 1-10 in which the selected Mfw gene is deactivated by inhibition by expression of RNAi.
12. A method as paragraphed in any of paragraphs 1-7, wherein the selected Mfw gene is deactivated by non-transgenic mutagenesis.
13. A wheat plant or seed that is male-sterile as a result of deactivation of one or more Mfw and/or Mpew genes.
14. A population of wheat plants that is predominantly male-sterile as a result of deactivation of one or more Mfw and/or Mpew genes.
15. A plant, seed, or population of wheat plants as paragraphed in paragraphs 13-14 in which one or more of the Mfw and/or Mpew genes deactivated is listed in Table 1 or Table 2.
16. A plant, seed, or population of wheat plants as paragraphed in paragraph 13-15 in which one or more of the Mfw and/or Mpew genes deactivated code for an amino-acid sequence having at least 60%, preferably at least 90% or 95% identity with any of SEQ ID NOs 1-6 and/or 30-35.
17. A population of wheat plants as paragraphed in any of paragraphs 13-16 that is at least 50%, preferably at least 90%, particularly 97% male-sterile.
18. A population of wheat plants as paragraphed in any of paragraphs 13-17 that is at least 97% male-sterile.
19. A population of wheat plants as paragraphed in any of paragraphs 13-18 which is substantially genetically uniform.
20. A plant, seed, or population of any of paragraphs 13-19, wherein the one or more Mfw and/or Mpew genes are at least two of Mfw1, Mfw2, Mfw3, and Mfw5.
21. A male-sterile wheat plant comprising deactivating modifications of each of the six copies of one or more Mfw and/or Mpew genes.
22. The male-sterile wheat plant of paragraph 21, wherein the deactivating modification is identical across the three genomes.
23. The male-sterile wheat plant of paragraph 21, wherein each genome comprises a different deactivating modification.
24. The male-sterile wheat plant of any of paragraphs 21-23, wherein one or more of the Mfw and/or Mpew genes deactivated is listed in Table 1 or Table 2.
25. The male-sterile wheat plant of any of paragraphs 21-24, wherein one or more of the Mfw and/or Mpew genes code for an amino-acid sequence having at least 60%, preferably at least 90% or 95% identity with any of SEQ ID NOs 1-6 and/or 30-35.
26. The male-sterile wheat plant of any of paragraphs 21-25, wherein the Mfw and/or Mpew gene is Mfw1, Mfw2, Mfw3, or Mfw 5.
27. The male-sterile wheat plant of any of paragraphs 21-26, wherein the one or more Mfw and/or Mpew gene is at least two of Mfw1, Mfw2, Mfw3, or Mfw5.
28. A hybrid wheat plant and/or seed comprising at least one deactivated copy of a Mfw and/or Mpew gene and at least one wild-type copy of the same Mfw and/or Mpew gene.
29. A population of hybrid wheat plants comprising at least one deactivated copy of a Mfw and/or Mpew gene and at least one wild-type copy of the same Mfw and/or Mpew gene.
30. The plant, seed, or population of any of paragraphs 28-29, wherein the one or more Mfw and/or Mpew genes are at least two of Mfw1, Mfw2, Mfw3, and Mfw5.
31. The plant, seed, or population of any of paragraphs 13-30, wherein the deactivating modification is a site-directed mutagenic event resulting from the activity of a site-specific nuclease; or the at least one Mfw and/or Mpew gene is deactivated by site-directed mutagenesis resulting from the activity of a site-specific nuclease.
32. The plant, seed, or population of paragraph 31, wherein the site-specific nuclease is CRISPR-Cas.
33. The plant, seed, or population of any of paragraphs 13-30, wherein
   the deactivating modification is excision of at least part of a coding or regulatory sequence; or
   the at least one Mfw and/or Mpew gene is deactivated by excision of at least part of a coding or regulatory sequence.
34. The plant, seed, or population of any of paragraphs 13-30, wherein the deactivating modification is insertion of RNAi-encoding sequences; or the at least one Mfw and/or Mpew gene is deactivated by inhibition by expression of RNAi.
35. The plant, seed, or population of any of paragraphs 13-30, wherein
   the deactivating modification is non-transgenic mutagenesis; or
   the at least one Mfw and/or Mpew gene is deactivated by non-transgenic mutagenesis.
36. A process of obtaining wheat hybrids which comprises crossing a wheat plant or population of wheat plants paragraphed in any of paragraphs 13-35 with male-fertile wheat.
37. A process paragraphed in paragraph 36 which comprises crossing a population paragraphed in any of paragraphs 13-35 with a uniform population of male-fertile wheat.
38. Hybrids produced by the process of either of paragraphs 36 or 37.
39. A plant, seed, or population of wheat plants comprising: a) a deactivating modification of each nuclear copy of one or more Mfw and/or Mpew genes; and
   b) a nucleic acid encoding an exogenous wild-type sequence of at least one of the Mfw and/or Mpew genes, wherein the nucleic acid is located in the cytoplasmic genome.
40. A plant, seed, or population of wheat plants comprising:
   a. a deactivating modification of each nuclear copy of one or more Mfw and/or Mpew genes; and
   b. a Mfw/PD/HT construct;
   wherein the Mfw/PD/HT construct is introgressed into the genome of the plant, seed, or population of plants; and
   whereby the plant, seed, or population of plants can pollinate a male-sterile plant comprising the deactivating modifications of clause a., but not the construct of clause
   b., resulting in male-sterile seed and/or progeny plants which are isogenic with the male-sterile plant.
41. The plant, seed, or population of wheat plants of any of paragraphs 39-40, wherein the one or more Mfw and/or Mpew genes are at least two of Mfw1, Mfw2, Mfw3, and Mfw 5.
42. The plant, seed, or population of wheat plants of any of paragraphs 39-41, further comprising a selectable marker gene or selectable marker construct.

### EXAMPLES

### Example 1

mRNAseq (as described in Trapnell et al., 2011) was used on wheat. The objective is to produce a set of ESTs (expressed sequence tags) from the RNA seq reads to discover genes expressed during flower development. This set of ESTs will contain both full length and fragments of genes. Arranging matching overlaps (using suitable software) allows the coding sequences of (most or all of) the expressed genes to be deduced.

Material was collected from stamens and pistils of immature flowers (at or around the time of meiosis and gamete development) and RNA was extracted from each tissue type.

Total RNA was extracted from three biologically replicated samples of developing stamens and pistils of wheat (Triticum aestivum) plants, cultivar Fielder. Tissues were selected and dissected from wheat ears between the Zadok stages 41-49 and total RNA was isolated using Qiagen's RNeasy® kit. Samples were then treated with DNAse to remove any further genomic contamination and purified using RNeasy Minelute® columns. Six RNA Seq libraries (three from stamens and three from pistils) were generated and sequenced using an Illumina HiSeq 2500 150 base pair paired end reads. These cDNA libraries were treated with the enzyme Ribo Zero (Illumina) to reduce the abundance of ribosomal RNAs before the libraries were run on the Illumina HiSeq2500. Sequencing was performed by Eurofins Genomics.

Obtained reads from the six libraries were analyzed using the bioinformatics software tool ‘fastQC’ to identify adapter contamination (available on the world wide web at bioinformatics.babraham.ac.uk/projects/fastqc/). Adapter contamination was removed from the reads using the ‘cutadapt’ software and trimmed sequences were again run through fastQC to assure adapters had been removed. Trimmed reads were aligned to the Chapman et al. Genome release using the ‘cufflinks’ suite of bioinformatics tools to determine differences in expression of genes between the two tissue types (Trapnell et al., 2011). Differentially expressed transcripts were run through ‘Blast2GO’ (bioinformatics platform) for a reference annotation (Conesa, et al., 2005).

A reference transcriptome was built using ‘cufflinks’ to allow the identification of candidate genes.

Sequencing results were compared to released wheat sequences as given in Chapman et al (2014) and TGAC genomes to understand gene models and fill any gaps in sequence knowledge (downloadable from The Genome Analysis Centre, Norwich, January 2016, ensemblgenomes.org/pub/plants/pre/fasta/triticum_aestivum/dna/). The sequences provided in Clavijo et al, (2016) can also be used in a similar fashion.

As noted above, wheat has an estimated 104,000 protein-coding genes, see Clavijo et al, (2016). The transcriptome analysis of this Example gave 8471 genes or gene fragments differentially expressed in the immature pistils or stamens analysed. Of these, 6668 were expressed higher in the stamen tissues: 6149 genes or gene fragments were expressed in the stamen only; 519 were expressed in the stamen and pistil with the stamen expression being higher than the pistil expression by factors ranging from 133 (102.29 Fragments Per Kilobase of transcript per Million [FPKM] in the stamen compared to 0.7657 FPKM in the pistil) to 8.6 (8.7895 FPKM in the stamen to 1.024 in the pistil).

The 6668 genes and gene fragments expressing in the stamens were then aligned to the TGAC genome released in January 2016 to validate their sequence (eliminating or combining gene fragments into single genes) and find their locus (including which chromosome) and show which of these genes have homology with genes found and described in other species. Genes having homology with genes from other species previously described as being involved with pollen development were selected for further analysis. This further analysis was based on i) degree of confidence in inferring function of the genes (based on their sequence available, their level of conserved sequence [at least 45% similarity] in comparison with putatively homologous genes in other plant species and a demonstrated link with male-fertility. in such other species) and ii) evidence of homoeologous copies in at least two, preferably three out of the three wheat genomes. This analysis and structured selection process gave a number of genes as candidates for further test. These are shown in Table 1 and Table 2.

TABLE 1

| | Cross | | | | | |
|---|---|---|---|---|---|---|
| Assigned | reference | | | Pistil | Stamen | |
| Mfwname | to Table 2 | Blast hit | Associated transcript | expression | expression | Homoeologues |
| Mfw1-D | a | RPG1 (RUPTURED POLLEN | Traes_7DL_015FBEE0C.1 | 0 | 5.8181 | Traes_7AL_F9E72D016.1 |
| | | GRAIN1) like | | | | Traes_7BL_784EA335F.1 |
| Mwf4-D | b | RPG1 (RUPTURED POLLEN | Traes_5DS_17EB80BAC.1 | 0 | 3.354 | Traes_5AS_2BDDAC590.2 |
| | | GRAIN1) like | | | | Traes_5BS_33597360E.1 |
| Mfw3-B | c | Aborted microspore 1 like | Traes_6AS_884A8FA55 | 0.666759 | 22.5582 | Traes_6BS_63C2E4C75 |
| Mfw3-A | d | Aborted microspore 1 like | Traes_6BS_63C2E4C75 | 0.994102 | 44.3336 | Traes_6AS_884A8FA55 |
| Mfw5-A | e | bHLH91 | Traes_2AL_FAB4B4A20 | 0.305494 | 12.6531 | Traes_2BL_1DDA22EA3 |
| | | | | | | Traes_2DL_9DD224B48 |
| Mfw5-D | f | bHLH91 | Traes_2DL_9DD224B48 | 1.35188 | 18.6834 | Traes_2BL_D3FAA4D64 |
| | | | | | | Traes_2AL_6FC5F1FD0 |
| Mfw2-B | g | callose synthase 5 | Traes_7BS_170A2F4BB.1 | 0 | 724.068 | Traes_7AS_AC78A59B0.2 |
| | | | | | | Traes_7DS_3B08482C9.1 |
| Mfw2-B | h | callose synthase 5 | Traes_7BS_170A2F4BB.1 | 0 | 14.3366 | Traes_7AS_AC78A59B0.2 |
| | | | | | | Traes_7DS_3B08482C9.1 |
| Mfw2-D | i | callose synthase 5 | Traes_7DS_674C1055E.2 | 0 | 36.152 | Traes_7BS_170A2F4BB.1 |
| | | | | | | Traes 7AS AC78A59B0.2 |
| Mfw2-B | j | callose synthase 5 like | Traes_7BS_170A2F4BB.1 | 0 | 2.04192 | Traes_7AS_AC78A59B0.2 |
| | | | | | | Traes_7DS_3B08482C9.1 |
| Mfw2-B | k | callose synthase 5 like | Traes_7DS_674C1055E.2 | 0 | 2.10844 | Traes_7BS_170A2F4BB.l |
| | | | | | | Traes 7AS AC78A59B0.2 |
| Mfw6-A | l | GAMYB (AtMYB101) | Traes_6AS_5562B97F7 | 1.17948 | 53.1065 | Traes_6DS_A0EC5D808.1 |
| Mfw6-A | m | GAMYB (AtMYB101) | Traes_6AS_5562B97F7.1 | 0 | 1.188 | Traes_6DS_A0EC5D808.1 |
| Mfw6-D | n | GAMYB (AtMYB101) | Traes_6DS_A0EC5D808.1 | 0 | 1.8778 | Traes_6AS_5562B97F7.1 |
| Mfw6-D | o | GAMYB (AtMYB101) | Traes_6DS_A0EC5D808.1 | 0 | 5.26915 | Traes_6AS_5562B97F7.1 |
| Mfw6-D | p | Hothead | Traes_1DL_4C479DE73 | 1.68379 | 54.1902 | Traes_1BL_CF9A1EAC4 |
| | | | | | | Traes_1AL_E0F69742D |
| Mfw7-B | q | Hothead | Traes_4BL_96CA397DA | 0.181023 | 24.1233 | Traes_4DL_1049A91B7 |
| Mfw7-B | r | Hothead | Traes_4BL_96CA397DA | 0.204306 | 27.0504 | Traes_4DL_0A4D9B04E |
| Mfw7-D | s | Hothead | Traes_4DL_1049A91B7 | 0 | 6.95578 | Traes_4BL_96CA397DA |
| Mfw8-D | t | Hothead | Traes_6DL_0CA2DAF56 | 0 | 296.701 | no strong hit |
| Mfw9-B | u | member of the sweet family | Traes_2BS_E686AA452.1 | 0 | 19.5814 | Traes_2DS_0F3296166 |
| Mfw10-A | v | member of the sweet family | Traes_7AS_43647E27C.1 | 1.63664 | 17.2441 | Traes_7BS_D48ECD082.1 |
| | | | | | | Traes_7DS_F2ACF99D2.1 |
| Mfw11-B | w | Similar to OsSweet7e | Traes_5BL_DE3 86929C.1 | 0 | 0.959594 | no strong hit |
| Mfw12-D | x | Sweet4 | Traes_1DL_9AC3057FA.1 | 0 | 4.17944 | Traes_1AL_D06B0BF4E.1 |
| Mfw1-A | y | RPG1 (RUPTURED POLLEN | Traes_7AL_F9E72D016.1 | 0 | 0.323 | Traes_7BL_784EA335F.1; |
| | | GRAIN1) like | | | | Traes_7DL_015FBEE0C.1 |
| Mfw1-B | z | RPG1 (RUPTURED POLLEN | Traes_7BL_784EA335F.1 | 0 | 0.110322 | Traes_7BL_784EA335F.1; |
| | | GRAIN1) like | | | | Traes_7DL_015FBEE0C.1 |
| Mfw2-A | aa | callose synthase 5 (see. e.g., | Traes_7AS_AC78A59B0.2 | 0.471962 | 12.939 | Traes_7BS_170A2F4BB.1; |
| | | SEQ ID NOs 4, 10, 14) | | | | Traes_7DS_674C1055E.2 |
| Mfw3-D | ab | Aborted microspore 1 like | not in IWGSC v 26 | 0.608498 | 12.285 | Traes_6AS_884A8FA55; |
| | | | | | | Traes_6BS_63C2E4C75 |
| Mfw5-B | ac | bHLH91 | not in IWGSC v 26 | 1.75714 | 18.2507 | Traes_2AL_FAB4B4A20; |
| | | | | | | Traes_2DL_9DD224B48 |

TABLE 2

| | Table 1 | | | | TGAC v1 gene model* - the closest match on a |
|---|---|---|---|---|---|
| Assigned | Cross- | Pistil | Stamen | | particular genome for the sequence/contig we built |
| Mfw name | reference | expression | expression | Blast hit | using our RNA data |
| Mfw1-A | y | 0 | 0.323 | RPG1 | TRIAE_CS42_7AL_TGACv1_556969_AA1774370 |
| | | | | (RUPTURED | |
| | | | | POLLEN | |
| | | | | GRAIN1) like | |
| Mfw1-B | z | 0 | 0.110322 | RPG1 | TRIAE_CS42_7BL_TGACv1_580455_AA1914070 |
| | | | | (RUPTURED | |
| | | | | POLLEN | |
| | | | | GRAIN1) like | |
| Mfw1-D | a | 0 | 5.8181 | RPG1 | TRIAE_CS42_7DL_TGACv1_603435_AA1983700 |
| | | | | (RUPTURED | |
| | | | | POLLEN | |
| | | | | GRAIN1) like | |
| Mfw2-A | aa | 0.471962 | 12.939 | callose | TRIAE_CS42_7AS_TGACv1_569258_AA1811650 |
| | | | | synthase 5 | |
| Mfw2-B | g | 0 | 724.068 | callose | TRIAE_CS42_7BS_TGACv1_593715_AA1953990 |
| | | | | synthase 5 | |
| ″ | h | 0 | 14.3366 | callose | ″ |
| | | | | synthase 5 | |
| ″ | j | 0 | 2.04192 | callose | ″ |
| | | | | synthase 5 | |
| ″ | k | 0 | 2.10844 | callose | ″ |
| | | | | synthase 5 | |
| Mfw2-D | i | 0 | 36.152 | callose | TRIAE_CS42_7DS_TGACv1_622598_AA2042310 |
| | | | | synthase 5 | |
| Mfw3-A | d | 0.994102 | 44.3336 | Aborted | TRIAE_CS42_6AS_TGACv1_486918_AA1566480 |
| | | | | microspore 1 | |
| | | | | like | |
| Mfw3-B | c | 0.666759 | 22.5582 | Aborted | TRIAE_CS42_6BS_TGACv1_514404_AA1659330 |
| | | | | microspore 1 | |
| | | | | like | |
| Mfw3-D | ab | 0.608498 | 12.285 | Aborted | TRIAE_CS42_U_TGACv1_643846_AA2135420 |
| | | | | microspore 1 | |
| | | | | like | |
| Mfw4-D | b | 0 | 3.354 | RPG1 | TRIAE_CS42_5BS_TGACv1_423307_AA1373980; |
| | | | | (RUPTURED | |
| | | | | POLLEN | |
| | | | | GRAIN1) like | |
| Mfw5-A | e | 0.305494 | 12.6531 | bHLH91 | TRIAE_CS42_2AL_TGACv1_094707_AA0301850 |
| Mfw5-B | ac | 1.75714 | 18.2507 | bHLH91 | TRIAE_CS42_2BL_TGACv1_129925_AA0399500 |
| Mfw5-D | f | 1.35188 | 18.6834 | bHLH91 | TRIAE_CS42_2DL_TGACv1_158620_AA0523420 |
| Mfw6-A | l | 1.17948 | 53.1065 | GAMYB | TRIAE_CS42_6AS_TGACv1_485682_AA1550030 |
| | | | | (AtMYB101) | |
| ″ | m | 0 | 1.188 | GAMYB | ″ |
| | | | | (AtMYB101) | |
| Mfw6-D | n | 0 | 1.8778 | GAMYB | TRIAE_CS42_6DS_TGACv1_543879_AA1744870 |
| | | | | (AtMYB101) | |
| ″ | o | 0 | 5.26915 | GAMYB | ″ |
| | | | | (AtMYB101) | |
| Mfw7-B | q | 0.181023 | 24.1233 | Hothead | TRIAE_CS42_4BL_TGACv1_320326_AA1035360 |
| ″ | r | 0.204306 | 27.0504 | ″ | ″ |
| Mfw7-D | s | 0 | 6.95578 | Hothead | TRIAE_CS42_4DL_TGACv1_343496_AA1135340 |
| Mfw8-D | t | 0 | 296.701 | Hothead | TRIAE_CS42_6DL_TGACv1_527115_AA1698830 |
| Mfw9-B | u | 0 | 19.5814 | member of the | TRIAE_CS42_2DS_TGACv1_177708_AA0582810 |
| | | | | sweet family | |
| Mfw10-A | v | 1.63664 | 17.2441 | member of the | TRIAE_CS42_7AS_TGACv1_570345_AA1834200 |
| | | | | sweet family | |
| Mfw11-B | w | 0 | 0.959594 | Similar to | TRIAE_CS42_U_TGACv1_640821_AA2075730 |
| | | | | OsSweet7e | |
| Mfw12-D | x | 0 | 4.17944 | Sweet4 | TRIAE_CS42_1DL_TGACv1_065128_AA0236610 |
| Mfw13-D | p | 1.68379 | 54.1902 | Hothead | TRIAE_CS42_1DL_TGACv1_063432_AA0227210 |

| Assigned | TGAC v1 homoeologues* - the copies on the other sub |
|---|---|
| Mfw name | genomes of wheat and their associated gene models |
| Mfw1-A | TRIAE_CS42_7BL_TGACv1_580455_AA1914070; |
| | TRIAE_CS42_7DL_TGACv1_603435_AA1983700 |
| Mfw1-B | TRIAE_CS42_7AL_TGACv1_556969_AA1774370; |
| | TRIAE_CS42_7DL_TGACv1_603435_AA1983700 |
| Mfw1-D | TRIAE_CS42_7AL_TGACv1_556969_AA1774370; |
| | TRIAE_CS42_7BL_TGACv1_580455_AA1914070 |
| Mfw2-A | TRIAE_CS42_7BS_TGACv1_593715_AA1953990; |
| | TRIAE_CS42_7DS_TGACv1_622598_AA2042310 |
| Mfw2-B | TRIAE_CS42_7AS_TGACv1_569258_AA1811650; |
| | TRIAE_CS42_7DS_TGACv1_622598_AA2042310 |
| ″ | TRIAE_CS42_7AS_TGACv1_569258_AA1811650; |
| | TRIAE_CS42_7DS_TGACv1_622598_AA2042310 |
| ″ | TRIAE_CS42_7AS_TGACv1_569258_AA1811650; |
| | TRIAE_CS42_7DS_TGACv1_622598_AA2042310 |
| ″ | TRIAE_CS42_7AS_TGACv1_569258_AA1811650; |
| | TRIAE_CS42_7DS_TGACv1_622598_AA2042310 |
| Mfw2-D | TRIAE_CS42_7BS_TGACv1_593715_AA1953990; |
| | TRIAE_CS42_7AS_TGACv1_569258_AA181165 |
| Mfw3-A | TRIAE_CS42_6BS_TGACv1_514404_AA1659330; |
| | TRIAE_CS42_U_TGACv1_643846_AA2135420 |
| Mfw3-B | TRIAE_CS42_6AS_TGACv1_486918_AA1566480; |
| | TRIAE_CS42_U_TGACv1_643846_AA2135420 |
| Mfw3-D | TRIAE_CS42_6AS_TGACv1_486918_AA1566480; |
| | TRIAE_CS42_6BS_TGACv1_514404_AA1659330 |
| Mfw4-D | TRIAE_CS42_5AS_TGACv1_393366_AA1271880; |
| | TRIAE_CS42_5DS_TGACv1_457788_AA1489840 |
| Mfw5-A | TRIAE_CS42_2BL_TGACv1_129925_AA0399500; |
| | TRIAE_CS42_2DL_TGACv1_158620_AA0523420 |
| Mfw5-B | TRIAE_CS42_2AL_TGACv1_094707_AA0301850; |
| | TRIAE_CS42_2DL_TGACv1_158620_AA0523420 |
| Mfw5-D | TRIAE_CS42_2AL_TGACv1_094707_AA0301850; |
| | TRIAE_CS42_2BL_TGACv1_129925_AA0399500 |
| Mfw6-A | TRIAE_CS42_6DS_TGACv1_543879_AA1744870 |
| ″ | ″ |
| Mfw6-D | TRIAE_CS42_6AS_TGACv1_485682_AA1550030 |
| ″ | ″ |
| Mfw7-B | TRIAE_CS42_4DL_TGACv1_343496_AA1135340; |
| | TRIAE_CS42_5AL_TGACv1_375593_AA1224180 |
| ″ | TRIAE_CS42_4DL_TGACv1_343496_AA1135340; |
| | TRIAE_CS42_5AL_TGACv1_375593_AA1224180 |
| Mfw7-D | TRIAE_CS42_4BL_TGACv1_320326_AA1035360; |
| | TRIAE_CS42_5AL_TGACv1_375593_AA1224180 |
| Mfw8-D | TRIAE_CS42_6AL_TGACv1_470984_AA1500160; |
| | TRIAE_CS42_6BL_TGACv1_500863_AA1610910 |
| Mfw9-B | TRIAE_CS42_2AS_TGACv1_113352_AA0354890; |
| | TRIAE_CS42_2BS_TGACv1_149844_AA0497680 |
| Mfw10-A | TRIAE_CS42_7BS_TGACv1_591914_AA1925470 |
| Mfw11-B | no strong hit |
| Mfw12-D | TRIAE_CS42_1AL_TGACv1_002319_AA0040790; |
| | TRIAE_CS42_1BL_TGACv1_030610_AA0095680 |
| Mfw13-D | TRIAE_CS42_1AL_TGACv1_001690_AA0034080; |
| | TRIAE_CS42_1BL_TGACv1_032570_AA0131570 |

*Clavijo et al (2016) and associated public access wheat genome database
**In the event of a conflict of gene designations, the Mfw names assigned in Table 2 will be controlling.

Further explanation of the headings in Table 1
‘Blast hits’—Best DNA sequence hit found with the BLAST2GO program
‘Associated transcript’—Refers to the best associated gene model aligned to the IWGSC genome. The name given in the column may be located online at plants.ensembiorg/Triticum_aestivum/Transcript/ . . . Version 28
Pistil expression and Stamen expression—given in FPKM units
Homoeologues—Under this heading are listed the best predictions of the homoeologues on the other genomes of wheat and their associated gene model using the IWGSC (International Wheat Genome Sequencing Consortium) models.

Table 1 references sequence information available on the world-wide web from the International Wheat Genome Sequencing Consortium's database, whereas Table 2 presents sequence information available on the world-wide web from The Genome Analysis Centre's database (Clavijo et al, 2016). The genes in Tables 1 and 2 are cross-referenced for clarity.

Of the genes in Tables 1 and 2, six (Mfw1-A, Mfw1-B, Mfw1-D, Mfw2-A, Mfw2-B and Mfw2-D) were chosen for RNAi knockout in Example 2.

Genes of interest were identified where expression is high in stamens and low or undetectable in pistils. The genes selected and specifically identified in this patent had the following expression levels: Mfw1-A, Stamen 2.36796.FPKM, Pistil 0.016006.FPKM; Mfw1-B, Stamen 3.15965.FPKM, Pistil 0.132269.FPKM; Mfw1-D Stamen 5.8181.FPKM, Pistil 0.FPKM; Mfw2-A Stamen 16.2411.FPKM, Pistil 0.362906.FPKM; Mfw2-B Stamen 724.068.FPKM, Pistil 0 FPKM; Mfw2-D Stamen 36.152.FPKM, Pistil 0.FPKM. No genes were selected which had expression only or predominantly in the pistil.

### Example 2

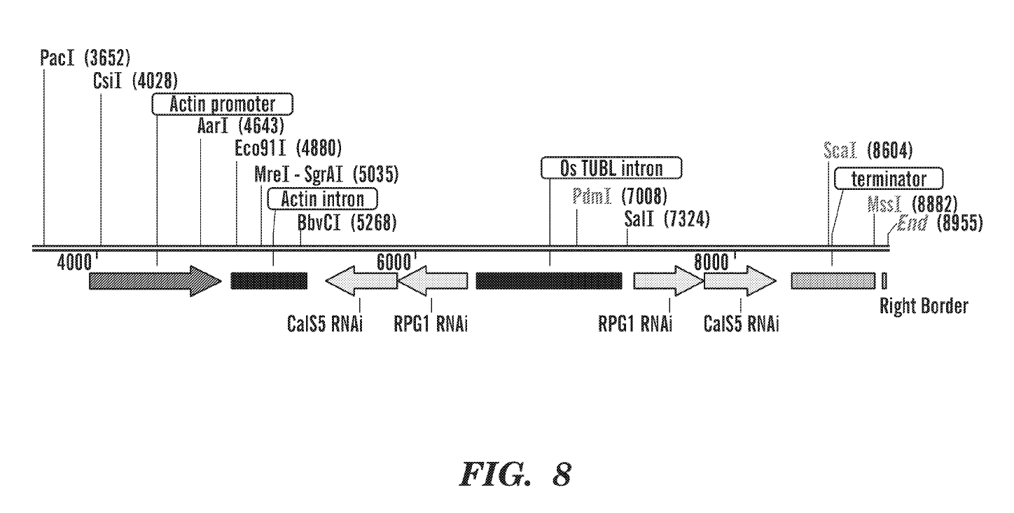

To produce a construct that would inhibit expression of two genes required for male fertility in wheat, a hairpin molecule was designed to target six of the Mfw genes identified in Example 1 above, and to inhibit them by RNAi. The hairpin molecule is formed from two targeting sequences joined end to end, as shown in SEQ ID NO 19. This chimeric sequence comprises 450 bp from the coding sequence for Mfw1-A (bases 1 to 450 as shown in SEQ ID NO 7 linked to 450 bp from the sequence for Mfw2-A (bases 1169 to 1619 as shown in SEQ ID NO 10). To generate inhibiting RNAi, the chimeric SEQ ID NO 19 is inserted in a construct in two copies, one 5′-3′ and one 3′-5′, separated by an intron spacer (see FIG. 8). When transcribed, this construct forms a hairpin molecule in which the two chimeric sequences are the limbs of the hairpin and the intron spacer is the joining loop. This hairpin is then processed by the cell machinery to form inhibiting RNAi. The two halves of the chimeric sequence SEQ ID NO 19 match exactly part of the coding sequences of Mfw1-A and Mfw2-A, so inhibiting these genes. They are also sufficiently similar to the corresponding coding sequences of Mfw1-B,D and Mfw2-B,D so as at to inhibit expression of the latter as well. The construct devised in order to generate the SEQ ID NO 19 hairpin is an insert about 9,000 bases long, shown diagrammatically in FIGS. 7 and 8. FIG. 7 shows the first 3,800 bases of the construct, 5′ to 3′, including the left border, the Sc4 promoter for the selection gene at about 500 to 1,000 basepairs, the FAD intron at about 1,000 to 2,300 base pairs, and the nptII selection gene from around 2,300 to 3,200 base pairs. A terminator is included at 3,300 to 3,500 base pairs. FIG. 7 shows the remaining 5,200 bases of the construct, including the rice actin promoter (McElroy et al (1990)) at 4,000 to 4,700 base pairs and the actin intron at 4,900 to 5,300 base pairs. This is followed by the chimeric insert SEQ ID NO 19 (inserted 3′ to 5′), from 5,500 to 6,400 base pairs; the Os TUBL intron, as separator, from 6,400 to 7,300 base pairs and then the chimeric insert SEQ ID NO 19 (this time 5′ to 3′) from 7,300 to 8,200 base pairs, followed by a terminator sequence and the right border. This construct is transformed into wheat by the method described in Example 3 below.

### Example 3

Wheat transformation of Fielder spring wheat germplasm with the construct prepared in Example 2 was carried out using immature wheat embryos, following Ishida et al. (2015). Tissue culture steps using media and nptII selection and plantlet regeneration were carried out as in Risacher et al (2009). The resulting insert in the wheat genome generates an RNAi hairpin molecule that inhibits expression of one or more Mfw genes (Mfw1 and Mfw2) in the transformed plants. Transformed plants are then grown to seed and their fertility assessed by comparing their overall pollen viability with known male-fertile ‘Fielder’ wheat plants which express Mfw1 and Mfw2 normally.

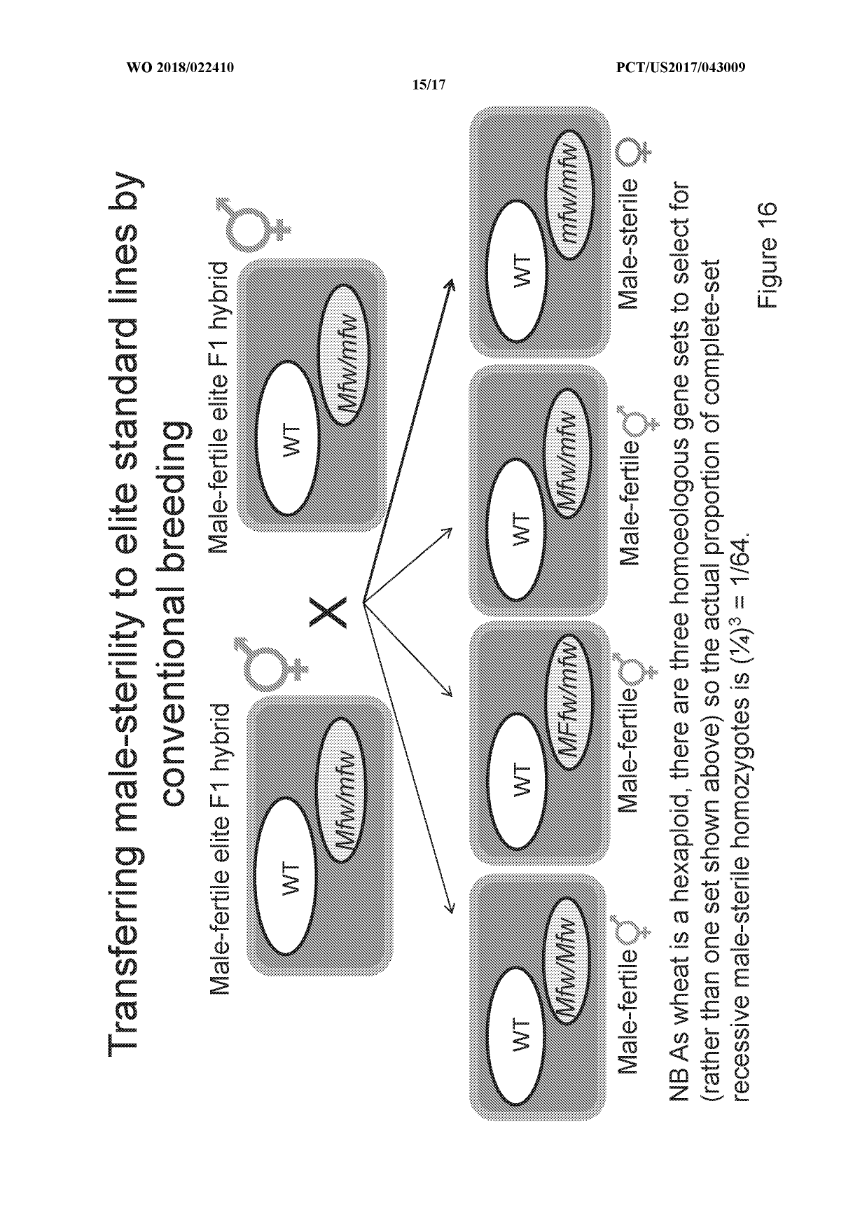

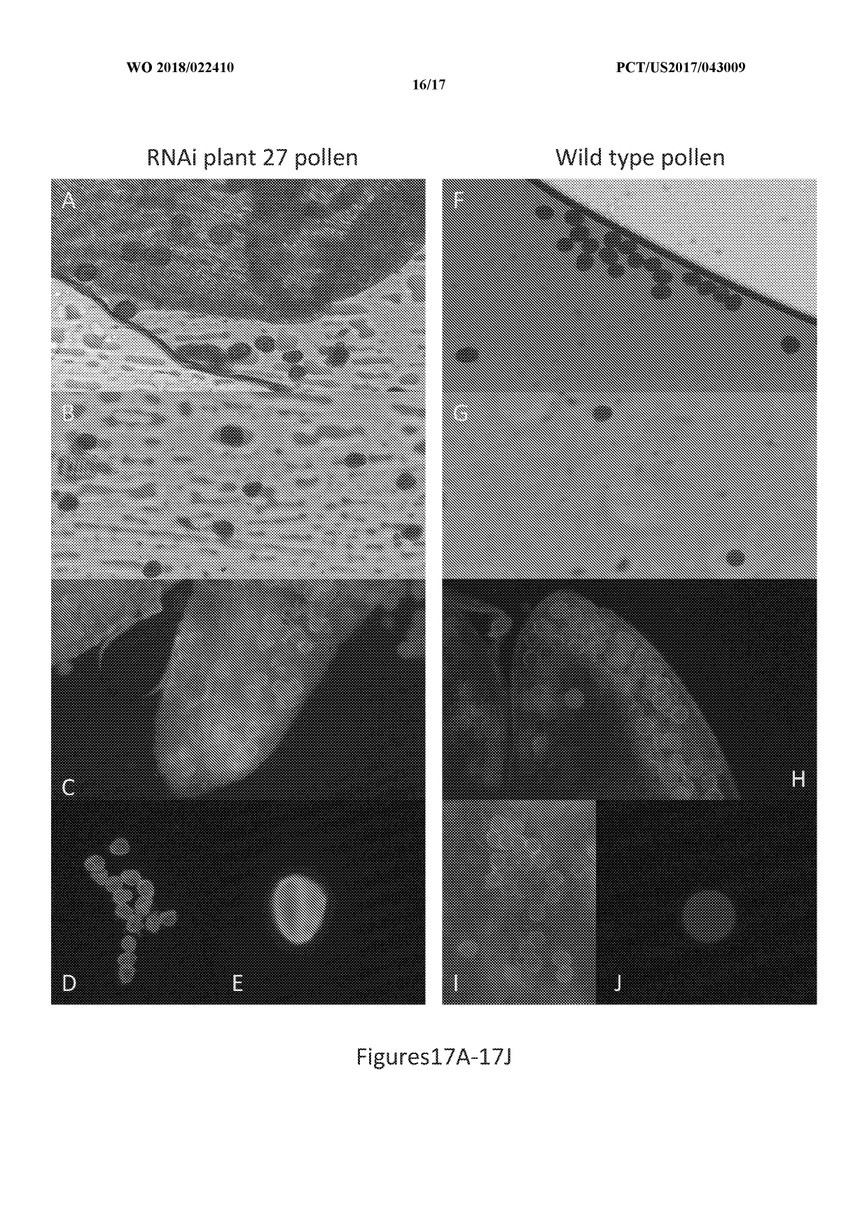

Forty transgenic plants containing an RNAi construct as described above, e.g. targeting 450 bases of both Mfw1 and Mfw2 genes, were generated and grown to seed. Overall, plants containing the RNAi construct were similar to wild-type plants with no observable differences seen in traits such as height, flowering time, leaf angle or leaf number. To assess the pollen specific phenotypes, pollen samples were taken from three anthers of each plant and stained with Alexander stain to assess pollen viability. All 40 of the plants suggested viable pollen with the Alexander stain. However, pollen from plant 27 looked malformed and misshapen (FIGS. 17A-17J). Pollen from plant 27, which has 4 or more copies of the RNAi construct, was than stained with Auramine 0 to gain better distinction of the pollen. Pollen from two plants (9 and 27) showed abnormal pollen when stained with Auramine 0 (FIGS. 17A-17J). Pollen from these two plants were invaginated and deflated compared to well-filled spheres in the case of pollen from wild-type plants. Upon further analysis, flowers of these two plants were not pollinated (ie not self-pollinated) by the time of anther extrusion and appeared to be male sterile. Further examination of flowers from plants 9 and 27 showed normal female flower parts and crossing some of the flowers from plants 9 or 27 with wild-type pollen led to the formation of seeds; thus both plants were female-fertile. The flowers of plants 9 and 27 which were not hand-cross-pollinated remained unfertilized and developed no embryos or seed; thus they were completely male-sterile.

### Example 4

To produce plants with targeted mutations in Mfw1 and Mfw2 we used a CRISPR Cas system to introduce mutations in wheat plants. We targeted Mfw1 and Mfw2 with four guide RNAs for each set of homoeologues. To identify the target sequences in these genes we used the publicly available program DREG (available on the world wide web at emboss.sourceforge.net/apps/cvs/emboss/apps/dreg.html) to find sequences that match either ANNNNNNNNNNNNNNNNNNNNGG or GNNNNNNNNNNNNNNNNNNNNGG in both directions of the Fielder genomic sequence. We then selected four guides based on the following criteria: that the target sequence was conserved in all three homoeologues, that it was (at least partially) in an exon of Mfw1 or Mfw2, that it had a restriction enzyme site near the site of the protospacer associated motif (PAM) but in the sequence of the guide RNA and prioritized guides near the start of the coding sequences of each gene. We also sought to use both AN20GG and GN20GG as this would stabilize the construct for transformation in the plant. The guide sequences selected are shown as SEQ ID NOs: 22-29. For targeting Mfw2 (CalS5-like) we drove one guide by the OsU3, TaU3, TaU6 and OsU6 promoters for a total of four guides targeting Mfw2. For targeting Mfw1 (RPG1-like) we repeated the TaU6 promoter as we could not find a sequence in the Mfw1 gene that could fill all of our criteria for quality guides. These two promoter guides constructs were then synthesized by Genscript and subsequently cloned into an intermediate vector containing L1 L5r flanking sites for Gateway Multisite recombination (Petersen & Stowers, 2011) into the final binary vector containing a wheat-optimized Cas9 enzyme driven by the maize ubiquitin promoter flanked by L5 and L2 sites. This final vector was introduced into Agrobacterium for transformation into wheat using the method as described in Example 3. Plants were then screened for mutations using a PCR based method where the PCR product was digested with an appropriate enzyme previously identified to cut the DNA at a site near the PAM. PCR products which are not cut therefore contain a mutation induced by the CRISPR construct. If no restriction enzyme site existed in a region targeted (for example, Mfw2 Guide 3 below) then direct sequencing of the PCR product was used to determine if a mutation exists.

By way of non-limiting example, the following enzymes are suitable for use with the guide sequences described below herein:

| | Suitable Enzyme |
|---|---|
| Mfw1 Guide | |
| Guide 1 (SEQ ID NO: 22) | HpyAV |
| Guide 2 (SEQ ID NO: 23) | MbiI |
| Guide 3 (SEQ ID NO: 24) | AjII |
| Guide 4 (SEQ ID NO: 25) | Eco105I |
| Mfw2 Guide | |
| Guide 1 (SEQ ID NO: 26) | BpiI/BtsIMutI |
| Guide 2 (SEQ ID NO: 27) | MscI |
| Guide 3 (SEQ ID NO: 28) | |
| Guide 4 (SEQ ID NO: 29) | BglI |

Exemplary guide sequences are depicted within the context of SEQ ID NOs 20-21 below and are individually identified, in order, as SEQ ID NOs 22-29.

-Sequence for Mfw1 guides (guide targeting sequences
shown in bold (SEQ ID NOs: 22-25, in order))
SEQ ID NO: 20
CAAATAATGATTTTATTTTGACTGATAGTGACCTGTTCGTTGCAACAAATTGATG
AGCAATGCTTTTTTATAATGCCAAGTTTGTACAAAAAAGCAGGCTTTAACCGCGG
TATACAAGGAATCTTTAAACATACGAACAGATCACTTAAAGTTCTTCTGAAGCAA
CTTAAAGTTATCAGGCATGCATGGATCTTGGAGGAATCAGATGTGCAGTCAGGG
ACCATAGCACAAGACAGGCGTCTTCTACTGGTGCTACCAGCAAATGCTGGAAGC
CGGGAACACTGGGTACGTTGGAAACCACGTGATGTGAAGAAGTAAGATAAACTG
TAGGAGAAAAGCATTTCGTAGTGGGCCATGAAGCCTTTCAGGACATGTATTGCA
GTATGGGCCGGCCCATTACGCAATTGGACGACAACAAAGACTAGTATTAGTACC
ACCTCGGCTATCCACATAGATCAAAGCTGATTTAAAAGAGTTGTGCAGATGATCC
GTGGCATCGGGAATGTCATCTCCTTGTTTTAGAGCTAGAAATAGCAAGTTAAA
ATAAGGCTAGTCCGTTATCAACTTGAAAAAGTGGCACCGAGTCGGTGCTTTTTTT
TATAACTTAAGCCGCGGGTATACTTAATTAAATTGGATGATCTGATAATTAACCC
GGGGACCAAGCCCGTTATTCTGACAGTTCTGGTGCTCAACACATTTATATTTATC
AAGGAGCACATTGTTACTCACTGCTAGGAGGGAATCGAACTAGGAATATTGATC
AGAGGAACTACGAGAGAGCTGAAGATAACTGCCCTCTAGCTCTCACTGATCTGG
GTCGCATAGTGAGATGCAGCCCACGTGAGTTCAGCAACGGTCTAGCGCTGGGCT
TTTAGGCCCGCATGATCGGGCTTTTGTCGGGTGGTCGACGTGTTCACGATTGGGG
AGAGCAACGCAGCAGTTCCTCTTAGTTTAGTCCCACCTCGCCTGTCCAGCAGAGT
TCTGACCGGTTTATAAACTCGCTTGCTGCATCAGACTTGTACGTACCATGATGG
TGAGGTTTTAGAGCTAGAAATAGCAAGTTAAAATAAGGCTAGTCCGTTATCAAC
TTGAAAAAGTGGCACCGAGTCGGTGCTTTTTTTCCGCGGCACGTCTCGAGCCCGG
GTTAATTAAATTGGATGATGACTCTAGATAACGCAGAAGATTAATTAACCCGGG
GACCAAGCCCGTTATTCTGACAGTTCTGGTGCTCAACACATTTATATTTATCAAG
GAGCACATTGTTACTCACTGCTAGGAGGGAATCGAACTAGGAATATTGATCAGA
GGAACTACGAGAGAGCTGAAGATAACTGCCCTCTAGCTCTCACTGATCTGGGTC
GCATAGTGAGATGCAGCCCACGTGAGTTCAGCAACGGTCTAGCGCTGGGCTTTTA
GGCCCGCATGATCGGGCTTTTGTCGGGTGGTCGACGTGTTCACGATTGGGGAGAG
CAACGCAGCAGTTCCTCTTAGTTTAGTCCCACCTCGCCTGTCCAGCAGAGTTCTG
ACCGGTTTATAAACTCGCTTGCTGCATCAGACTTGATCATCAAGGCCAAGGACG
GTTTTAGAGCTAGAAATAGCAAGTTAAAATAAGGCTAGTCCGTTATCAACTTGAA
AAAGTGGCACCGAGTCGGTGCTTTTTTTCCGCGGCACGTCTCGAGCCCGGGTTAA
TTAAATTGGATGATGACTCTAGATAACGCAGGATCCACTAGTAACGGCCGCCAG
TGTGCTGGAATTGCCCTTGGATCATGAACCAACGGCCTGGCTGTATTTGGTGGTT
GTGTAGGGAGATGGGGAGAAGAAAAGCCCGATTCTCTTCGCTGTGATGGGCTGG
ATGCATGCGGGGGAGCGGGAGGCCCAAGTACGTGCACGGTGAGCGGCCCACAG
GGCGAGTGTGAGCGCGAGAGGCGGGAGGAACAGTTTAGTACCACATTGCCCAGC
TAACTCGAACGCGACCAACTTATAAACCCGCGCGCTGTCGCTTGTGTGGGGGAT
GGGGGCTTACGTAGTTTTAGAGCTAGAAATAGCAAGTTAAAATAAGGCTAGTC
CGTTATCAACTTGAAAAAGTGGCACCGAGTCGGTGCTTTTTTTGTCCCTTCGAAG
GGCAATTCTGCAGATATCCATCACACTGGCGGCCGCTCGAGGTCGAGGGTATCG
ATAAGCTTGAATTCGACCCAGCTTTCTTGTACAAAGTTGGCATTATAAAAAATAA
TTGCTCATCAATTTGTTGCAACGAACAGGTCACTATCAGTCAAAATAAAATCATT
ATTTG
-Sequence for Mfw2 guides (guide targeting sequences
shown in bold (SEQ ID NOs: 26-29 in order))
SEQ ID NO: 21
CAAATAATGATTTTATTTTGACTGATAGTGACCTGTTCGTTGCAACAAATTGATG
AGCAATGCTTTTTTATAATGCCAAGTTTGTACAAAAAAGCAGGCTTTAACCGCGG
TATACAAGGAATCTTTAAACATACGAACAGATCACTTAAAGTTCTTCTGAAGCAA
CTTAAAGTTATCAGGCATGCATGGATCTTGGAGGAATCAGATGTGCAGTCAGGG
ACCATAGCACAAGACAGGCGTCTTCTACTGGTGCTACCAGCAAATGCTGGAAGC
CGGGAACACTGGGTACGTTGGAAACCACGTGATGTGAAGAAGTAAGATAAACTG
TAGGAGAAAAGCATTTCGTAGTGGGCCATGAAGCCTTTCAGGACATGTATTGCA
GTATGGGCCGGCCCATTACGCAATTGGACGACAACAAAGACTAGTATTAGTACC
ACCTCGGCTATCCACATAGATCAAAGCTGATTTAAAAGAGTTGTGCAGATGATCC
GTGGCACACCTGATTGTTTCTCACTGTTTTAGAGCTAGAAATAGCAAGTTAAAA
TAAGGCTAGTCCGTTATCAACTTGAAAAAGTGGCACCGAGTCGGTGCTTTTTTTT
ATAACTTAAGCCGCGGGTATACTTAATTAAATTGGATGATCTGACTAGATACCGG
TCTCGAGTTAACATGAATCCAAACCACACGGAGTTCAAATTCCCACAGATTAAG
GCTCGTCCGTCGCACAAGGTAATGTGTGAATATTATATCTGTCGTGCAAAATTGC
CTGGCCTGCACAATTGCTGTTATAGTTGGCGGCAGGGAGAGTTTTAACATTGACT
AGCGTGCTGATAATTTGTGAGAAATAATAATTGACAAGTAGATACTGACATTTGA
GAAGAGCTTCTGAACTGTTATTAGTAACAAAAATGGAAAGCTGATGCACGGAAA
AAGGAAAGAAAAAGCCATACTTTTTTTTAGGTAGGAAAAGAAAAAGCCATACGA
GACTGATGTCTCTCAGATGGGCCGGGATCTGTCTATCTAGCAGGCAGCAGCCCTA
CCAACCTCACGGGCCAGCAATTACGAGTCCTTCTAAAACGTCCCGCCGAGGGCG
CGTGGCCGTGCTGTGCAGCAGCACGTCTAACATTAGTCCCACCTCGCCAGTTTAC
AGGGAGCAGAACCAGCTTATAAGCGGAGGCGCGGCACCAAGAAGCAACTTGCA
TCTAATGTGGCCGTTTTAGAGCTAGAAATAGCAAGTTAAAATAAGGCTAGTCCG
TTATCAACTTGAAAAAGTGGCACCGAGTCGGTGCCAACATTTTTTTTGTCCTTCTG
TTTTTTTAGTCAGTCTCTTTTTTCAGAAGTACAACATCTTTTTTTTGTCCTTCTGTT
TTTTTAGTCAGTCTTTTTTCAGAAGTACTCTATGTGATATCTTCGTTCTGGGAAAT
GTCTGTCTGTCTACAACCCATAATTATATTTGCAATCACACATCTAATATCTCTGT
GACAAGACAGCCGAACAACCTAGGTAAGATTAATTAACCCGGGGACCAAGCCCG
TTATTCTGACAGTTCTGGTGCTCAACACATTTATATTTATCAAGGAGCACATTGTT
ACTCACTGCTAGGAGGGAATCGAACTAGGAATATTGATCAGAGGAACTACGAGA
GAGCTGAAGATAACTGCCCTCTAGCTCTCACTGATCTGGGTCGCATAGTGAGATG
CAGCCCACGTGAGTTCAGCAACGGTCTAGCGCTGGGCTTTTAGGCCCGCATGATC
GGGCTTTTGTCGGGTGGTCGACGTGTTCACGATTGGGGAGAGCAACGCAGCAGT
TCCTCTTAGTTTAGTCCCACCTCGCCTGTCCAGCAGAGTTCTGACCGGTTTATAAA
CTCGCTTGCTGCATCAGACTTGGATGGCCAATGCGAGATGAGTTTTAGAGCTAG
AAATAGCAAGTTAAAATAAGGCTAGTCCGTTATCAACTTGAAAAAGTGGCACCG
AGTCGGTGCTTTTTTTCCGCGGCACGTCTCGAGCCCGGGTTAATTAAATTGGATG
ATGACTCTAGATAACGCAGGATCCACTAGTAACGGCCGCCAGTGTGCTGGAATT
GCCCTTGGATCATGAACCAACGGCCTGGCTGTATTTGGTGGTTGTGTAGGGAGAT
GGGGAGAAGAAAAGCCCGATTCTCTTCGCTGTGATGGGCTGGATGCATGCGGGG
GAGCGGGAGGCCCAAGTACGTGCACGGTGAGCGGCCCACAGGGCGAGTGTGAG
CGCGAGAGGCGGGAGGAACAGTTTAGTACCACATTGCCCAGCTAACTCGAACGC
GACCAACTTATAAACCCGCGCGCTGTCGCTTGTGTGATAGTAGTTAGTGCCGCG
TGTTTTAGAGCTAGAAATAGCAAGTTAAAATAAGGCTAGTCCGTTATCAACTTGA
AAAAGTGGCACCGAGTCGGTGCTTTTTTTGTCCCTTCGAAGGGCAATTCTGCAGA
TATCCATCACACTGGCGGCCGCTCGAGGTCGAGGGTATCGATAAGCTTGAATTCG
ACCCAGCTTTCTTGTACAAAGTTGGCATTATAAAAAATAATTGCTCATCAATTTG
TTGCAACGAACAGGTCACTATCAGTCAAAATAAAATCATTATTTG
SEQ ID NO: 22
TCGGGAATGTCATCTCCTT
SEQ ID NO: 23
TACGTACCATGATGGTGAG
SEQ ID NO: 24
ATCATCAAGGCCAAGGACG
SEQ ID NO: 25
GGGGATGGGGGCTTACGTA
SEQ ID NO: 26
CACCTGATTGTTTCTCACT
SEQ ID NO: 27
ACTTGCATCTAATGTGGCC
SEQ ID NO: 28
GATGGCCAATGCGAGATGA
SEQ ID NO: 29
ATAGTAGTTAGTGCCGCGT

The individual T₀ CRISPR-transformed plants had genomic DNA isolated from leaf tissue taken before flowering-time and this was analysed for both large deletions, smaller deletions, indels, or SNPs using the four restrictions enzyme sites designed into the guide. These enzymes include MbiI, AjiI and Eco105I for Mfw1 sequences and BpiI, MlsI or BglI for Mfw2. From the results of these assays, it was established which plants had missense mutations at any or all Mfw loci. The results were then considered to decide which plants had complementary deletions and such plants were cross-pollinated onto some but not all of the flowers of the relevant plants. In the case where all three loci or either Mfw1 or Mfw2 were mutated, apparently male-sterile flowers were crossed to wild-type pollen to ensure that the sterility was male sterility only and not complete sterility. Some flowers were left un-crossed to ensure that the pollinated flowers which appeared male-sterile at flowering were in fact male-sterile at maturity. Embryos were then excised from the fertilised flowers (reference for wheat embryo rescue needed here) to produce T₁ plantlets and, where embryos not taken, seed from the fertilised flowers was then sown in order to produce T₁ plants which were tested, using the same procedure as before, to find those which had combined significant deletions in all six homoeologous copies of the Mfw gene concerned. Those which did have such deletions and were male-sterile were cross-pollinated with others which were male-fertile but had the highest number of deletions. In such a way a population is produced which includes some males-steriles. With repetition of this process, further male-steriles can be produced until a separately-produced maintainer-line is established to effect larger-scale production of the male-sterile line.

### Example 5

A male-sterile wheat plant produced according to the method described in Example 4 is grown to flower maturity and fertilised with pollen of the wheat variety ‘Sadash’. Seed sets, and is collected from the plant. In this way is obtained a population consisting of fertile F₁ hybrid wheat seeds, substantially uniform in phenotypic expression, and typically displaying hybrid vigour.

### Example 6

To produce a construct that would inhibit expression of two genes required for male fertility in wheat, a hairpin molecule was designed to target six of the Min) genes identified in Example 1 above, and to inhibit them by RNAi. The hairpin molecule is formed from two targeting sequences joined end to end, as shown in SEQ ID NO 48. This chimeric sequence comprises 450 bp from the coding sequence for Mfw5-A (bases 207 to 656 as shown in SEQ ID NO 7 linked to 450 bp from the sequence for Mfw3-B (bases 100 to 549 as shown in SEQ ID NO 48). To generate inhibiting RNAi, the chimeric SEQ ID NO 48 is inserted in a construct in two copies, one 5c-3′ and one 3′-5′, separated by an intron spacer (see FIG. 8). When transcribed, this construct forms a hairpin molecule in which the two chimeric sequences are the limbs of the hairpin and the intron spacer is the joining loop. This hairpin is then processed by the cell machinery to form inhibiting RNAi. The two halves of the chimeric sequence SEQ ID NO 48 match exactly part of the coding sequences of Mfw5-A and Mfw3-B, so inhibiting these genes. They are also sufficiently similar to the corresponding coding sequences of Mfw5-B,D and Mfw3-A,D so as at to inhibit expression of the latter as well.

The construct devised in order to generate the SEQ ID NO 48 hairpin is an insert about 9,000 bases long. It follows the same plan used for the construct to generate the insert SEQ ID NO 19 in Examples 2 and 3. This plan is as shown diagrammatically in FIGS. 7 and 8. FIG. 7 shows the first 3,800 bases of the construct, 5′ to 3′, including the left border, the Sc4 promoter for the selection gene at about 500 to 1,000 basepairs, the FAD intron at about 1,000 to 2,300 basepairs, and the nptII selection gene from around 2,300 to 3,200 basepairs. A terminator is included at 3,300 to 3,500 basepairs. FIG. 7 shows the remaining 5,200 bases of the construct, including the rice actin promoter (McElroy et al (1990)) at 4,000 to 4,700 basepairs and the actin intron at 4,900 to 5,300 basepairs. This is followed by the chimeric insert SEQ ID NO 48 (inserted 3′ to 5′), from 5,500 to 6,400 basepairs; the OsTUBL intron, as separator, from 6,400 to 7,300 basepairs and then the chimeric insert SEQ ID NO 48 (this time 5′ to 3′) from 7,300 to 8,200 basepairs, followed by a terminator sequence and the right border. This construct is transformed into wheat by the method described in Example 7 below.

### Example 7

Wheat transformation of Fielder spring wheat germplasm with the construct prepared in Example 6 is carried out using immature wheat embryos, following Ishida et al. (2015), Tissue culture steps using media and nptII selection and plantlet regeneration is carried out as in Risacher et al (2009). The resulting insert in the wheat genome generates an RNAi hairpin molecule that inhibits expression of one or more Mfw genes (Mfw3 and Mfw5) in the transformed plants. Transformed plants are then grown to seed and their fertility assessed by comparing their overall pollen viability with known male-fertile ‘Fielder’ wheat plants which express Mfw3 and Mfw5 normally.

### REFERENCES

Belhaj et al., (2015), “Editing plant genomes with CRISPR/Cas9”, Current Opinion in Biotechnology, vol 32, pp 6-84
Belhaj et al. (2013): Plant genome editing made easy: targeted mutagenesis in model and crop plants using the CRISPR/Cas system. Plant Methods 2013 9:39;
Bogdanove et al. (2011) Science 333:1843-6;
Carlson D F, Tan W, Lillico S G, Stverakova D, Proudfoot C, Christian M. et al. Efficient TALEN-mediated gene knockout in livestock. Proc Natl Acad Sci USA. (2012); 109:17382-7. doi:10.1073/pnas.1211446109,
Carroll (2013). “Staying on target with CRISPR-Cas”. Nature Biotechnology. 31 (9), p 807-809
Chapman et al, (2015) “A whole genome shotgun approach for assembling and anchoring the hexaploid bread wheat genome”, Genome Biology, 16 (26), pp 1-17
Clavijo et al (2016) “An improved assembly and annotation of the allohexaploid wheat genome identifies complete families of agronomic genes and provides genomic evidence for chromosomal translocations,” Cold Spring Harbor Laboratory non-reviewed pre-print. doi:http://dx.doi.org/10.1101/080796.
Conesa et al., (2005) “Blast2GO: a universal tool for annotation, visualization and analysis in functional genomics research”, Bioinformatics, vol. 21, pp 3674-3676 Dong et al., (2005) “Callose synthase (CalS5) is required for exine formation during microgametogenesis and for pollen formation in Arabidopsis”, Plant Journal 42: 315-328
Guerts et al. (2009) Science 325:433-3;
Harrison et al., (2015) “Using RNA Sequencing and in Silico Subtraction to Identify Resistance Gene Analog Markers for Lr16 in Wheat”, The Plant Genome, vol. 8, no, 2. pp 1-9
Ishida et al, (2015), Agrobacterium Protocols: Volume 1, Methods in Molecular Biology, vol. 1223, pp 189-198. Springer,
Jinek et al. (2012) Science 337:816-821
Jing, Bing, Shuangping Heng, Dan Tong, Zhengjie Wan, Tingdong Fu, Jinxing Tu, Chaozhi Ma, Bin Yi, Jing Wen, and Jinxiong Shen. (2012). ‘A Male Sterility-Associated Cytotoxic Protein ORF288 in Brassica Juncea Causes Aborted Pollen Development’. Journal of Experimental Botany 63 (3): 1285-95. doi:10.1093/jxb/err355.
Kim and Kim. (2014) Nature Reviews Genetics 15:321-334;
Kim et al. (2012) Genome Res. 22:1327-1333;
McElroy et al., (1990) “Isolation of an Efficient Actin Promoter for Use in Rice Transformation”, The Plant Cell, Vol. 2, pp 163-171.
Petersen L K, Stowers R S (2011) A Gateway MultiSite Recombination Cloning Toolkit. PLoS ONE 6(9): e24531.doi: 10.1371/journal.pone.0024531
Ran et al. (2013) Cell 2013 154:1380-9;
Risacher et al., (2009) “Highly efficient Agrobacterium—mediated transformation of wheat via in planta inoculation” in Jones, H. and Shewry, P. (eds), Methods in Molecular Biology, Transgenic Wheat, Barley and Oats', vol. 478, p 115-124, Humana Press, Springer
Shan et al, (2014) “Protocol Genome editing in rice and wheat using the CRISPR/Cas system”, Nature Protocols, 9, pp. 2395-2410
Silva, George, Laurent Poirot, Roman Galetto, Julianne Smith, Guillermo Montoya, Philippe Duchateau, and Frederic Paques. (2011) ‘Meganucleases and Other Tools for Targeted Genome Engineering: Perspectives and Challenges for Gene Therapy’. Current Gene Therapy 11 (1): 11-27. doi:10.2174/156652311794520111.
Sun M-X et al., (2013) “Arabidopsis RPG1 is important for primexine deposition and functions redundantly with RPG2 for plant fertility at the late reproductive stage”, Plant Reprod 26:83-91 DOI 10.1007/s00497-012-0208-1
Takasu, Yoko, Isao Kobayashi, Kelly Beumer, Keiro Uchino, Hideki Sezutsu, Suresh Sajwan, Dana Carroll, Toshiki Tamura, and Michal Zurovec. (2010). ‘Targeted Mutagenesis in the Silkworm Bombyx Mori Using Zinc Finger Nuclease mRNA Injection’. Insect Biochemistry and Molecular Biology 40 (10): 759-65. doi:10.1016/j.ibmb.2010.07.012.
Trapnell et al, (2013) “Differential analysis of gene regulation at transcript resolution with RNA-seq”, Nature Biotechnology, 2013 January, 31(1), doi:10.1038/nbt.2450.
Trick et at, (2012), “Combining SNP discovery from next-generation sequencing data with bulked segregant analysis (BSA) to fine-map genes in polyploid wheat”, BMC Plant Biology 2012, 12:14
Urnov et al. (2010) Nat Rev Genet 2010 11:636-646
Watanabe et al. (2012) Nat. Commun. 3;
Whitford et al., (2013) “Hybrid breeding in wheat: technologies to improve hybrid wheat seed production”, Journal of Experimental Botany, doi: 10.1093/jxb/ert333, pp 1-18
Zadoks et al., (1974) “A Decimal Code for the Growth Stages of Cereals”, Weed Research 14:415-421.
Zhang, Chunsheng, Kim H. Norris-Caneda, William H. Rottmann, Jon E. Gulledge, Shujun Chang, Brian Yow-Hui Kwan, Anita M. Thomas, et al. (2012). ‘Control of Pollen-Mediated Gene Flow in Transgenic Trees[W][OA]’. Plant Physiology 159 (4): 1319-34. doi:10.1104/pp. 112.197228.

1. A method of producing male-sterile wheat which comprises during the development of the flower:
analysing the RNA-transcriptome of wheat stamen cells;
analysing the RNA-transcriptome of wheat pistil cells;
inhibiting expression of at least one Mfw gene which is preferentially expressed in stamens rather than pistils, so as to produce male-sterile wheat.

2. The method of claim 1, wherein the RNA-transcriptome analysis is carried out during meiosis.

3. The method of claim 1, wherein the RNA-transcriptome analysis is carried out between stages 41 to 49 of the Zadoks scale, inclusive.

4. The method of claim 1, wherein the RNA-transcriptome analysis is carried out in juvenile flowers comprising both immature stamens and pistils.

5. The method of claim 1, wherein the Mfw gene codes for an amino-acid sequence identical to, or having corresponding function and least 60%, with any of SEQ ID NOs 1-6, 7-12, 30-41, 129-130 or a sequence of a gene of Tables 1 or 2.

6. (canceled)

7. The method of claim 1, wherein the at least one Mfw genes are at least two of Mfw1, Mfw2, Mfw3, and Mfw5.

8. The method of claim 1, wherein the Mfw gene is deactivated by site-directed mutagenesis employing a site-specific nuclease.

9. The method of claim 8, wherein the site-specific nuclease is CRISPR-Cas.

10. The method of claim 1, wherein the Mfw gene is deactivated by excision of at least part of a coding or regulatory sequence, inhibition by expression of RNAi, or non-transgenic mutagenesis.

11. (canceled)

12. (canceled)

13. A wheat plant or seed that is male-sterile as a result of deactivation of one or more Mfw or Mpew genes.

14. (canceled)

15. The wheat plant or seed of claim 13, wherein one or more of the Mfw or Mpew genes deactivated is listed in Table 1 or Table 2 or codes for an amino acid sequence having at least 60% identity with any of SEQ ID NOs: 1-6 and 30-35.

16. (canceled)

17. (canceled)

18. (canceled)

19. (canceled)

20. The plant or seed of claim 13, wherein the one or more Mfw or Mpew genes are selected from Mfw1, Mfw2, Mfw3, and Mfw5.

21. The plant or seed of claim 13, comprising deactivating modifications of each of the six copies of the one or more Mfw and/or Mpew genes.

22. The wheat plant or seed of claim 21, wherein the deactivating modification is identical across the three genomes.

23. The wheat plant or seed of claim 21, wherein each genome comprises a different deactivating modification.

24. (canceled)

25. (canceled)

26. (canceled)

27. (canceled)

28. (canceled)

29. (canceled)

30. (canceled)

31. The wheat plant or seed of claim 13, wherein the deactivating modification is a site-directed mutagenic event resulting from the activity of a site-specific nuclease; or the at least one Mfw or Mpew gene is deactivated by site-directed mutagenesis resulting from the activity of a site-specific nuclease.

32. The wheat plant or seed of claim 31, wherein the site-specific nuclease is CRISPR-Cas.

33. The wheat plant or seed of claim 13, wherein
the deactivating modification is excision of at least part of a coding or regulatory sequence; or
the at least one Mfw or Mpew gene is deactivated by excision of at least part of a coding or regulatory sequence;
the deactivating modification is insertion of RNAi-encoding sequences; or
the at least one Mfw or Mpew gene is deactivated by inhibition by expression of RNAi;
the deactivating modification is non-transgenic mutagenesis; or
the at least one Mfw or Mpew gene is deactivated by non-transgenic mutagenesis.

34. (canceled)

35. (canceled)

36. (canceled)

37. (canceled)

38. (canceled)

39. A wheat plant or seed comprising:
a) a deactivating modification of each nuclear copy of one or more Mfw or Mpew genes; and
b) a nucleic acid encoding an exogenous wild-type sequence of at least one of the Mfw or Mpew genes, wherein the nucleic acid is located in the cytoplasmic genome.

40. (canceled)

41. The wheat plant or seed of claim 39, wherein the one or more Mfw or Mpew genes are at least two of Mfw1, Mfw2, Mfw3, Mfw5.

42. (canceled)
